(19) United States
(12) Patent Application Publication
(10) Pub. No.: US 2009/0170105 A1
(43) Pub. Date: Jul. 2, 2009

(54) Diagnostics for Aging-Related Dermatologic Disorders

(75) Inventors: Kenneth S. Kornman, Newton, MA (US); Leon Wilkins, North Andover, MA (US)

Correspondence Address: MINTZ, LEVIN, COHN, FERRIS, GLOVSKY AND POPEO, P.C, ONE FINANCIAL CENTER, BOSTON, MA, 02111, US

(73) Assignee: INTERLEUKIN GENETICS, INC., Waltham, MA (US)

(21) Appl. No.: 12/268,233

(22) Filed: Nov. 10, 2008

Related U.S. Application Data
(60) Provisional application No. 60/986,331, filed on Nov. 8, 2007.

(51) Int. Cl. C12Q 1/68

(52) U.S. Cl. 435/6

(57) ABSTRACT

The present invention provides methods for the early prediction of aging-related dermatologic conditions of including skin changes associated with intrinsic aging or skin damages caused by extrinsic aging such as photoaging. The present invention also provides kits for the early determination of the propensity to develop such disorder and conditions. The method consists of detecting the presence of one or more alleles of an IL-1 haplotype or pattern, specifically the IL-1RN (+2018) and the IL-1B (−511) loci. The presence of allele 2 at the IL-1RN (+2018) and the IL-1B (−511) loci indicates decreased risk for a early onset of aging related dermatologic conditions.

### RELATED APPLICATIONS

This application claims the benefit of U.S. Ser. No. 60/986,331, filed Nov. 8, 2007, which is incorporated herein by reference in its entirety.

### FIELD OF THE INVENTION

This application relates to a prognostic method based on polymorphisms in the IL-1 gene cluster.

### BACKGROUND

Genetics of the IL-1 Gene Cluster

The IL-1 gene cluster is on the long arm of chromosome 2 (2q13) and contains at least the genes for IL-1α (IL-1A), IL-1β (IL-1B), and the IL-1 receptor antagonist (IL-1RN), within a region of 430 Kb (Nicklin, et al. (1994) Genomics, 19: 382-4). The agonist molecules, IL-1α and IL-1β, have potent pro-inflammatory activity initiate many inflammatory cascades. Their actions, often via the induction of other cytokines such as IL-6 and IL-8, lead to activation and recruitment of leukocytes into damaged tissue, local production of vasoactive agents, fever response in the brain and hepatic acute phase response. All three IL-1 molecules bind to type I and to type II IL-1 receptors with varying affinities, but only the type I receptor transduces a signal to the interior of the cell. In contrast, the type II receptor is shed from the cell membrane and acts as a decoy receptor. The receptor antagonist and the type II receptor, therefore, are both anti-inflammatory in their actions.

Certain alleles from the IL-1 gene cluster are already known to be associated with particular disease states. For example, IL-1RN allele 2 has been shown to be associated with coronary artery disease (PCT/US/98/04725, and U.S. Ser. No. 08/813,456), osteoporosis (U.S. Pat. No. 5,698,399), nephropathy in diabetes mellitus (Blakemore, et al. (1996) Hum. Genet. 97 (3): 369-74), alopecia areata (Cork, et al., (1995) J. Invest. Dermatol. 104 (5 Supp.): 15S-16S; Cork et al. (1996) Dermatol Clin 14: 671-8), Graves disease (Blakemore, et al. (1995) J. Clin. Endocrinol. 80 (1): 111-5), systemic lupus erythematosus (Blakemore, et al. (1994) Arthritis Rheum. 37: 1380-85), lichen sclerosis (Clay, et al. (1994) Hum. Genet. 94: 407-10), and ulcerative colitis (Mansfield, et al. (1994) Gastoenterol. 106 (3): 637-42).

In addition, the IL-1A allele 2 from marker -889 and IL-1B (TaqI) allele 2 from marker +3954 have been found to be associated with periodontal disease (U.S. Pat. No. 5,686,246; Komman and diGiovine (1998) Ann Periodont 3: 327-38; Hart and Kornman (1997) Periodontol 2000 14: 202-15; Newman (1997) Compend Contin Educ Dent 18: 881-4; Kornnan et al. (1997) J. Clin Periodontol 24: 72-77). The IL-1A allele 2 from marker −889 has also been found to be associated with juvenile chronic arthritis, particularly chronic iridocyclitis (McDowell, et al. (1995) Arthritis Rheum. 38: 221-28). The IL-1B (TaqI) allele 2 from marker +3954 of IL-1B has also been found to be associated with psoriasis and insulin dependent diabetes in DR3/4 patients (di Giovine, et al. (1995) Cytokine 7: 606; Pociot, et al. (1992) Eur J. Clin. Invest. 22: 396-402). Additionally, the IL-1RN (VNTR) allele 1 has been found to be associated with diabetic retinopathy (see U.S. Ser. No. 09/037,472, and PCT/GB97/02790). Furthermore allele 2 of IL-1RN (VNTR) has been found to be associated with ulcerative colitis in Caucasian populations from North America and Europe (Mansfield, J. et al., (1994) Gastroenterology 106: 637-42). Interestingly, this association is particularly strong within populations of ethnically related Ashkenazi Jews (PCT WO97/25445).

Genotype Screening

Traditional methods for the screening of heritable diseases have depended on either the identification of abnormal gene products (e.g., sickle cell anemia) or an abnormal phenotype (e.g., mental retardation). These methods are of limited utility for heritable diseases with late onset and no easily identifiable phenotypes such as, for example, a predisposition to early aging. With the development of simple and inexpensive genetic screening methodology, it is now possible to identify polymorphisms that indicate a propensity to develop disease, even when the disease is of polygenic origin. The number of diseases that can be screened by molecular biological methods continues to grow with increased understanding of the genetic basis of multifactorial disorders.

Genetic screening (also called genotyping or molecular screening), can be broadly defined as testing to determine if a patient has mutations (or alleles or polymorphisms) that either cause or alter a disease state or are “linked” to the mutation causing or altering a disease state. Linkage refers to the phenomenon that DNA sequences which are close together in the genome have a tendency to be inherited together. Two sequences may be linked because of some selective advantage of co-inheritance. More typically, however, two polymorphic sequences are co-inherited because of the relative infrequency with which meiotic recombination events occur within the region between the two polymorphisms. The co-inherited polymorphic alleles are said to be in linkage disequilibrium with one another because, in a given human population, they tend to either both occur together or else not occur at all in any particular member of the population. Indeed, where multiple polymorphisms in a given chromosomal region are found to be in linkage disequilibrium with one another, they define a quasi-stable genetic “haplotype.” In contrast, recombination events occurring between two polymorphic loci cause them to become separated onto distinct homologous chromosomes. If meiotic recombination between two physically linked polymorphisms occurs frequently enough, the two polymorphisms will appear to segregate independently and are said to be in linkage equilibrium.

While the frequency of meiotic recombination between two markers is generally proportional to the physical distance between them on the chromosome, the occurrence of “hot spots” as well as regions of repressed chromosomal recombination can result in discrepancies between the physical and recombinational distance between two markers. Thus, in certain chromosomal regions, multiple polymorphic loci spanning a broad chromosomal domain may be in linkage disequilibrium with one another, and thereby define a broad-spanning genetic haplotype. Furthermore, where a disease-causing mutation is found within or in linkage with this haplotype, one or more polymorphic alleles of the haplotype can be used as a diagnostic or prognostic indicator of the likelihood of developing the disease. This association between otherwise benign polymorphisms and a disease-causing polymorphism occurs if the disease mutation arose in the recent past, so that sufficient time has not elapsed for equilibrium to be achieved through recombination events. Therefore identification of a human haplotype which spans or is linked to a disease-causing mutational change, serves as a predictive measure of an individual's likelihood of having inherited that disease-causing mutation. Importantly, such prognostic or diagnostic procedures can be utilized without necessitating the identification and isolation of the actual disease-causing lesion. This is significant because the precise determination of the molecular defect involved in a disease process can be difficult and laborious, especially in the case of multifactorial diseases such as inflammatory disorders.

Indeed, the statistical correlation between a disorder and an IL-1 polymorphism does not necessarily indicate that the polymorphism directly causes the disorder. Rather the correlated polymorphism may be a benign allelic variant which is linked to (i.e. in linkage disequilibrium with) a disorder-causing mutation which has occurred in the recent human evolutionary past, so that sufficient time has not elapsed for equilibrium to be achieved through recombination events in the intervening chromosomal segment. Thus, for the purposes of diagnostic and prognostic assays for a particular disease, detection of a polymorphic allele associated with that disease can be utilized without consideration of whether the polymorphism is directly involved in the etiology of the disease. Furthermore, where a given benign polymorphic locus is in linkage disequilibrium with an apparent disease-causing polymorphic locus, still other polymorphic loci which are in linkage disequilibrium with the benign polymorphic locus are also likely to be in linkage disequilibrium with the disease-causing polymorphic locus. Thus these other polymorphic loci will also be prognostic or diagnostic of the likelihood of having inherited the disease-causing polymorphic locus. Indeed, a broad-spanning human haplotype (describing the typical pattern of co-inheritance of alleles of a set of linked polymorphic markers) can be targeted for diagnostic purposes once an association has been drawn between a particular disease or condition and a corresponding human haplotype. Thus, the determination of an individual's likelihood for developing a particular disease of condition can be made by characterizing one or more disease-associated polymorphic alleles (or even one or more disease-associated haplotypes) without necessarily determining or characterizing the causative genetic variation.

IL-1 in Skin Function and Homeostasis

The protein products of the IL-1 gene cluster (both the two agonists IL-1α, IL-1β, and the receptor antagonist IL-1RN) play a pivotal in the control of inflammatory states and responses in mammalian skin (Kupper and Groves, 1995). Their modulation has also been directly demonstrated in the skin wound healing process (Bryan, et al. 2005). in vitro studies utilizing cultured human skin keratinocytes have demonstrated that exposure to UV-light modulates the expression of all three major IL-1 gene cluster products, indicating their role in response to a major factor in the appearance of aged skin (Garmyn, et al. 1992: Luo, et al. 2004). Other studies have shown that the permeability barrier of skin changes with aging, and that IL-1 gene products play a role in determining this barrier function abnormality (Ye, et al 2002). Thus, selective alterations in the IL-1 family of cytokines that occur with aging influence how this barrier property responds to perturbation, and defects in IL-1 signaling may therefore contribute to the skin permeability barrier abnormalities of aged skin. It is the integrity of the connective tissue (primarily collagen) of skin that most directly influences the appearance of lines and wrinkling. The synthesis and breakdown of these connective tissue proteins are primarily regulated by resident dermal fibroblast cells. It has been shown that exposure of these cells in vitro to either exogenous IL-1α or IL-1β increases their synthesis of Types I and III collagens (Goldring and Krane 1987).

Role of Collagens/MMP in Skin Wrinkling

The coordinated regulation of collagen synthesis and breakdown (controlled by matrix metalloproteinases or MMP's) determines the health and firmness of this skin connective tissue layer, which determines the incidence and severity of lines and wrinkles. Evidence suggests that the deterioration of the integrity of skin collagens is part of a natural aging process that is accelerated (photoaging) by sun (UV-light) exposure. The factors involved in natural skin aging may be somewhat different than those of photoaging. Studies have shown that the natural aging process decreases collagen synthesis and increases the expression of MMPs, whereas photoaging results in an increase of collagen synthesis and greater expression of MMPs (Chung, et al. 2001). The levels of MMP-1 and MMP-2 (which breakdown collagen) were higher in the dermis of photoaged skin than in naturally aged (routinely sun-protected) skin. Using a mouse model system, researchers have shown that topical application of a specific MMP inhibitor (MMP-2, MMP-9) can prevent UVB-induced basement membrane disruption and wrinkle formation (Inomata, et al. 2003). Thus, the role of these MMPs in the formation of skin wrinkling has been well established.

IL-1 Induces Increased Expression/Production of MMPs in Skin

Numerous studies have demonstrated the connection between IL-1 and the stimulation of MMP production and activity in skin. Mauviel, et al (1993) demonstrated that, while the cytokines IL-1β, TNF-α, lymphotoxin (LT), PDGF, and bFGF all stimulate fibroblasts to produce collagenase, only IL-1β, TNF-α, and LT are capable of stimulating the 92 kDa gelatinase. It is known that both the collagenases (MMP-1) and gelatinases (MMP-2, MMP-9) are important modulators of skin connective tissue integrity. Others studying dermal fibroblasts in vitro confirmed that IL-1β stimulates MMP-1 protein levels, but it showed no corresponding stimulation of the endogenous MMP inhibitor, TIMP-1 (Dasu, et al. 2003). Thus IL-1 has the capability of shifting skin to an enhanced collagen breakdown state. Studies have shown that both IL-1α and IL-1β are potent stimulators of MMP-1 (Rutter, et al. 1997), and that IL-1α can induce activation of MMP-9 in human skin (Han, et al. 2005). The coordinated role of both IL-1 and MMPs was demonstrated in human skin exposed to single or repeated UV-light dosages of 1 Minimal Erythemal Dose (MED) (Seite, et al. 2004). These investigators found a three-fold induction of MMP-2 expression after both single or repeated exposures (sustained response), and a significant increase in both IL-1α and IL-1β after a single 1 MED exposure.

Throughout this description, including the foregoing description of related art, any and all publicly available documents described herein, including any and all U.S. patents, are specifically incorporated by reference herein in their entirety. The foregoing description of related art is not intended in any way as an admission that any of the documents described therein, including pending United States patent applications, are prior art to the present invention. Moreover, the description herein of any disadvantages associated with the described products, methods, and/or apparatus, is not intended to limit the invention. Indeed, aspects of the invention may include certain features of the described products, methods, and/or apparatus without suffering from their described disadvantages.

The section headings are used herein for organizational purposes only, and are not to be construed as in any way limiting the subject matter described.

### SUMMARY

In general, the invention relates to the observation that certain IL-1 genotypes are indicators for a genetic influence on aging. In certain aspects, the present application relates to methods for determining a subject's susceptibility to the early onset or progression of aging-related dermatologic conditions (ARDD). In one aspect, a method of the invention comprises obtaining a nucleic acid sample from a subject, and testing for the presence of at least one ARDD-associated allele and/or the at the presence of least one ARDD-associated allele of an IL-1 haplotype or allelic pattern. In certain embodiments, aging-related dermatologic conditions of the invention include dermatologic disorders that include skin changes associated with intrinsic aging or skin damages caused by extrinsic aging such as photoaging.

Generally, the method of predicting increased risk for dermatologic disorders consists of detecting the presence of at least one copy of an allele selected from the group consisting of IL-1B (−511) allele 2 and IL-1RN (+2018) allele 2. Having one or more of these alleles indicates decreased risk for inflammation-based dermatologic disorders (such as deleterious response to sun exposure). Detecting alleles may be performed directly, by analyzing the DNA from the IL-1 region, or indirectly, by analyzing the RNA or protein products of the DNA.

In another embodiment, the invention can be described as the following: isolating nucleic acid from the patient, identifying one or more alleles present in the IL-1 gene cluster, and comparing the one or more alleles to a control sample. The control sample contains at least one allele from the IL-1 gene cluster known to be associated with dermatologic disorders. In a preferred embodiment, the control sample contains the IL-1B (−511) allele 2 and IL-1RN (+2018) allele 2. Similarity of the identified alleles from the subject to the control sample indicates the subject's predisposition to dermatologic disorders.

Another embodiment of the invention is a kit for the detection of an allele that is predictive of dermatologic disorders. The kit generally includes at least one oligonucleotide complementary to a DNA sequence in the IL-1 gene family; and a control sample. The control sample is an allele known to be associated with dermatologic disorders, as above. The kit may also include a DNA sampling means, a DNA purification means, and PCR reagents. Further, the oligonucleotide may contain a detectable label. Further, the kits may contain a pharmaceutical or cosmetic agent for the treatment of a dermatologic disorder.

In an additional aspect, the invention provides methods for screening test substances to identify a test substance that is likely to prevent or diminish the early onset of an aging-related dermatologic condition. Methods of the invention comprise contacting a cell containing DNA comprised of at least one ARDD-associated allele or allelic pattern with a test substance; and observing at least one biomarker in said subject, wherein a change in a biomarker from a ARDD-related phenotype to a non-ARDD-related phenotype identifies a test substance that is likely to prevent or diminish the early onset of aging-related dermatologic diseases and conditions.

In a further aspect, the invention provides a method for screening genes to identify a gene that is likely to prevent or diminish the early onset of an aging-related dermatologic condition in a subject, said method comprising contacting a cell containing DNA comprised of at least ARDD-associated allele or allelic pattern with a test gene under conditions causing the test gene to enter one or more of said cells; and observing at least one biomarker in said subject, wherein a change in a biomarker from a ARDD-related phenotype to a non-ARDD-related phenotype identifies a test gene that is likely to prevent or diminish the early onset of aging-related dermatologic diseases and conditions.

In yet another aspect, the invention provides methods of treating, including the prophylactic treatment, or diminishing the early onset of an aging-related dermatologic condition. In one embodiment, a subject is contacted with a substance or gene identified according to the methods described above.

In another aspect, the invention provides methods for determining the stage of an aging-related dermatologic condition in a subject. The methods comprise observing at least one biomarker identified according to the methods described above and determining the degree to which the biomarker evinces an aging-related dermatologic phenotype. The greater the degree to which the biomarker evinces an aging related-dermatologic phenotype, the later the stage of the aging-related dermatologic condition.

Other embodiments and advantages of the invention are set forth in part in the description which follows, and will be obvious from this description, or may be learned from the practice of the invention.

### BRIEF DESCRIPTION OF THE FIGURES

FIG. 1 shows the nucleic acid sequence for IL-1A (GEN X03833; SEQ ID No. 1).

FIG. 2 shows the nucleic acid sequence for IL-1B (GEN X04500; SEQ ID No. 2).

FIG. 3 shows the nucleic acid sequence for the secreted IL-1RN (GEN X64532; SEQ ID No. 3).

FIG. 4 shows the nucleic acid sequence for the intracellular IL-1RN (GEN X77090; SEQ ID No. 4).

### DETAILED DESCRIPTION OF THE PREFERRED EMBODIMENTS

### Interleukin-1 and Dermatologic Aging

The invention is based, in part, on the finding that an individual's IL-1 genotype influences the genetic and cellular aspects of dermatologic aging in that individual. For example, IL-1 alleles are associated with e aging-related dermatologic conditions. Aging-related dermatologic conditions include dermatologic disorders that include skin changes associated with intrinsic aging or skin damages caused by extrinsic aging such as photoaging.

In certain aspects, methods of the invention may be used to predict the likelihood of an individual developing an aging-related dermatologic disorder. In certain embodiments, the invention relates to the observation that a subject population having a certain IL-1 genotype will, on average, experience a greater propensity of developing a dermatologic disorder and, in certain instances, will experience a more rapid progression of dermatologic disorders. In other aspects, a subject's IL-1 genotype may be used to identify subjects that would be candidates for preventative therapy or an aggressive or early therapy.

In certain embodiments, dermatologic disorders include skin changes associated with intrinsic aging or skin damages caused by extrinsic aging such as photoaging. In preferred embodiments, dermatologic disorders include the following: skin disorders associated with disturbed keratinization, structural integrity, or inflammation; wrinkles; dry skin; ichthyosis; palmar and plantar hyperkeratosis; dandruff; Darier's disease; lichen simplex chronicus; keratoses; acne; psoriasis; eczema; pruritus; keratosis pilaris, including keratosis pilaris rubra (red, inflamed bumps), alba (rough, bumpy skin with no irritation), rubra faceii (reddish rash on the cheeks); lichen planus; actinic keratosis (also called solar keratosis, or AK); seborrheic keratosis; and skin cancer, including basal cell carcinoma and squamous cell carcinoma.

In certain embodiments, dermatologic disorders include cosmetic conditions or dermatological conditions including: disturbed keratinization, defective syntheses of dermal components, and changes associated with aging of skin, nail and hair; and those indications which include dryness or loss of integrity of skin, nail and hair; xerosis; ichthyosis; palmar and plantar hyperkeratoses; uneven and rough surface of skin, nail and hair; dandruff; Darier's disease; lichen simplex chronicus; keratoses; acne; pseudofolliculitis barbae; eczema; psoriasis; itchy scalp and skin; pruritus; warts; herpes; age spots; lentigines; melasmas; blemished skin; hyperkeratoses; hyperpigmented skin; abnormal or diminished syntheses of collagen, glycosaminoglycans, proteoglycans and elastin as well as diminished levels of such components in the dermis; stretch marks; skin lines; fine lines; wrinkles; thinning of skin, nail plate and hair; skin thickening due to elastosis of photoaging, loss or reduction of skin, nail and hair resiliency, elasticity and recoilability; lack of skin, nail and hair lubricants and luster; dull and older-looking skin, nail and hair; fragility and splitting of nail and hair; or combinations thereof.

### DEFINITIONS

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “an aberrant activity”, as applied to an activity of a polypeptide such as IL-1, refers to an activity which differs from the activity of the wild-type or native polypeptide or which differs from the activity of the polypeptide in a healthy subject. An activity of a polypeptide can be aberrant because it is stronger than the activity of its native counterpart. Alternatively, an activity can be aberrant because it is weaker or absent relative to the activity of its native counterpart. An aberrant activity can also be a change in an activity. For example an aberrant polypeptide can interact with a different target peptide. A cell can have an aberrant IL-1 activity due to overexpression or underexpression of an IL-1 locus gene encoding an IL-1 locus polypeptide.

An “Aging-related dermatologic disorder-associated phenotype” or ARDD-associated phenotype” is a phenotype of subjects or cells that is associated with an aging-related dermatologic disorder or associated with an increased likelihood of aging-related dermatologic disorders. An ARDD-associated phenotype is also any phenotype found in a subject or cell having an ARDD-associated allele, where such phenotype differs from that found in subjects or cells lacking an ARDD-associated allele. Such phenotypes encompass essentially any characteristic of a biomarker. An ARDD-associated phenotype may not be directly involved in ARDD but may nonetheless serve as an indicator for ARDD. A “non-ARDD-associated phenotype” is a phenotype that is not associated with aging-related dermatologic disorders or with an increased likelihood of developing aging-related dermatologic disorders.

An “ARDD therapeutic” refers to any agent that prevents or postpones the development or alleviates the symptoms of early onset of aging-related dermatologic conditions. An ARDD therapeutic can be a polypeptide, peptidomimetic, nucleic acid, other inorganic or organic molecule, or a nutraceutical, preferably a “small molecule”. Preferably an ARDD therapeutic can modulate at least one ARDD-associated phenotype. For example, an ARDD therapeutic may modulate an activity of an IL-1 polypeptide, e.g., interaction with an IL-1 receptor, by mimicking or potentiating (agonizing) or inhibiting (antagonizing) the effects of a naturally-occurring IL-1 polypeptide. An IL-1 agonist can be a wild-type IL-1 protein or derivative thereof having at least one bioactivity of the wild-type IL-1, e.g. receptor binding activity. An IL-1 agonist can also be a compound that upregulates expression of an IL-1 gene or which increases at least one bioactivity of an IL-1 protein. An agonist can also be a compound which increases the interaction of an IL-1 polypeptide with another molecule, e.g., an interleukin receptor. An IL-1 antagonist can be a compound which inhibits or decreases the interaction between an IL-1 protein and another molecule, e.g., a receptor, such as an IL-1 receptor. Accordingly, a preferred antagonist is a compound which inhibits or decreases binding to an IL-1 receptor and thereby blocks subsequent activation of the IL-1 receptor. An antagonist can also be a compound that downregulates expression of an IL-1 locus gene or which reduces the amount of an IL-1 protein present. The IL-1 antagonist can be a dominant negative form of an IL-1 polypeptide, e.g., a form of an IL-1 polypeptide which is capable of interacting with a target peptide, e.g., an IL-1 receptor, but which does not promote the activation of the IL-1 receptor. The IL-1 antagonist can also be a nucleic acid encoding a dominant negative form of an IL-1 polypeptide, an IL-1 antisense nucleic acid, or a ribozyme capable of interacting specifically with an IL-1 RNA. Yet other IL-1 antagonists are molecules which bind to an IL-1 polypeptide and inhibit its action. Such molecules include peptides, e.g., forms of IL-1 target peptides which do not have biological activity, and which inhibit binding by IL-1 to IL-1 receptors. Thus, such peptides will bind the active site of IL-1 and prevent it from interacting with target peptides, e.g., an IL-1 receptor. Yet other IL-1 antagonists include antibodies interacting specifically with an epitope of an IL-1 molecule, such that binding interferes with the biological function of the IL-1 locus polypeptide. In yet another preferred embodiment, the IL-1 antagonist is a small molecule, such as a molecule capable of inhibiting the interaction between an IL-1 polypeptide and a target IL-1 receptor. Alternatively, the small molecule can function as an antagonist by interacting with sites other than the IL-1 receptor binding site. An antagonist can be any class of molecule, including a nucleic acid, protein, carbohydrate, lipid or combination thereof, but for therapeutic purposes is preferably a small molecule.

The term “allele” refers to the different sequence variants found at different polymorphic regions. For example, IL-1RN (VNTR) has at least five different alleles. The sequence variants may be single or multiple base changes, including without limitation insertions, deletions, or substitutions, or may be a variable number of sequence repeats.

The term “allelic pattern” refers to the identity of an allele or alleles at one or more polymorphic regions. For example, an allelic pattern may consist of a single allele at a polymorphic site, as for IL-1RN (VNTR) allele 1, which is an allelic pattern having at least one copy of IL-1RN allele 1 at the VNTR of the IL-1RN gene loci. Alternatively, an allelic pattern may consist of either a homozygous or heterozygous state at a single polymorphic site. For example, IL-1RN (VNTR) allele 2,2 is an allelic pattern in which there are two copies of the second allele at the VNTR marker of IL-1RN and that corresponds to the homozygous IL-RN (VNTR) allele 2 state. Alternatively, an allelic pattern may consist of the identity of alleles at more than one polymorphic site.

The term “antibody” as used herein is intended to refer to a binding agent including a whole antibody or a binding fragment thereof which is specifically reactive with an IL-1B polypeptide. Antibodies can be fragmented using conventional techniques and the fragments screened for utility in the same manner as described above for whole antibodies. For example, F(ab)₂ fragments can be generated by treating an antibody with pepsin. The resulting F(ab)₂ fragment can be treated to reduce disulfide bridges to produce Fab fragments. The antibody of the present invention is further intended to include bispecific, single-chain, and chimeric and humanized molecules having affinity for an IL-1B polypeptide conferred by at least one CDR region of the antibody.

“Biological activity” or “bioactivity” or “activity” or “biological function”, which are used interchangeably, for the purposes herein means an effector or antigenic function that is directly or indirectly performed by an IL-1 polypeptide (whether in its native or denatured conformation), or by any subsequence thereof. These terms are also intended to encompass properties of IL-1 proteins and genes, such as expression levels and post-translational modifications. Biological activities include binding to a target peptide, e.g., an IL-1 receptor. An IL-1 bioactivity can be modulated by directly affecting an IL-1 polypeptide. Alternatively, an IL-1 bioactivity can be modulated by modulating the level of an IL-1 polypeptide, such as by modulating expression of an IL-1 gene.

As used herein the term “bioactive fragment of an IL-1 polypeptide” refers to a fragment of a full-length IL-1 polypeptide, wherein the fragment specifically mimics or antagonizes the activity of a wild-type IL-1 polypeptide. The bioactive fragment preferably is a fragment capable of interacting with an interleukin receptor.

The term “biomarker” refers to a phenotype of a subject or cells. Biomarkers encompass a broad range of intra- and extra-cellular events as well as whole organism physiological changes. Biomarkers may be any of these and are not necessarily involved in inflammatory responses. With respect to cells, biomarkers may be essentially any aspect of cell function, for example: levels or rate of production of signaling molecules, transcription factors, intermediate metabolites, cytokines, prostanoids, steroid hormones (e.g. estrogen, progesterone, androstenedione or testosterone), gonadotropins (e.g. LH and FSH), gene transcripts, post-translational modifications of proteins, gonadotropin releasing hormone (GnRH), catecholamines (e.g. dopamine or norepinephrine), opioids, activin, inhibin, as well as IL-1 bioactivities. Biomarkers may include whole genome analysis of transcript levels or whole proteome analysis of protein levels and/or modifications. Additionally, biomarkers may be reporter genes. For example, an IL-1 promoter or an IL-1 promoter comprising an ARDD-associated allele can be operationally linked to a reporter gene. In an alternative method, the promoter can be an IL-1-regulated promoter, such as IL-8. In this manner, the activity of the reporter gene is reflective of the activity of the promoter. Suitable reporter genes include GUS, LacZ, green fluorescent protein (GFP) (and variants thereof, such as Red Fluorescent Protein, Cyan Fluorescent Protein, Yellow Fluorescent Protein and Blue Fluorescent Protein), or essentially any other gene whose product is easily detected. Other preferred biomarkers include factors involved in immune and inflammatory responses, as well as factors involved in IL-1 production and signaling, as described below. In subjects, biomarkers can be, for example, any of the above as well as electrocardiogram parameters, pulmonary function, IL-6 activities, urine parameters or tissue parameters. “ARDD associated biomarkers” are any of the above which are found to correlate with ARDD, or which are preferentially found in subjects or cells comprising an ARDD-associated allele.

“Cells”, “host cells” or “recombinant host cells” are terms used interchangeably herein to refer not only to the particular subject cell, but to the progeny or potential progeny of such a cell. Because certain modifications may occur in succeeding generations due to either mutation or environmental influences, such progeny may not, in fact be identical to the parent cell, but is still included within the scope of the term as used herein.

A “chimera,” “mosaic,” “chimeric mammal” and the like, refers to a transgenic mammal with a knock-out or knock-in construct in at least some of its genome-containing cells.

The terms “comprise” and “comprising” is used in the inclusive, open sense, meaning that additional elements may be included.

The terms “control” or “control sample” refer to any sample appropriate to the detection technique employed. The control sample may contain the products of the allele detection technique employed or the material to be tested. Further, the controls may be positive or negative controls. By way of example, where the allele detection technique is PCR amplification, followed by size fractionation, the control sample may comprise DNA fragments of an appropriate size. Likewise, where the allele detection technique involves detection of a mutated protein, the control sample may comprise a sample of a mutant protein. However, it is preferred that the control sample comprises the material to be tested. For example, the controls may be a sample of genomic DNA or a cloned portion of the IL-1 gene cluster. However, where the sample to be tested is genomic DNA, the control sample is preferably a highly purified sample of genomic DNA.

A “clinical event” is an occurrence of clinically discernible signs of a disease or of clinically reportable symptoms of a disease. “Clinically discernible” indicates that the sign can be appreciated by a health care provider. “Clinically reportable” indicates that the symptom is the type of phenomenon that can be described to a health care provider. A clinical event may comprise clinically reportable symptoms even if the particular patient cannot himself or herself report them, as long as these are the types of phenomena that are generally capable of description by a patient to a health care provider.

A “dermatologic condition” or “aging-related dermatologic conditions” or “dermatologic disorder” refers to any skin disorder associated with aging or inflammation, which include skin changes associated with intrinsic aging or skin damages caused by extrinsic aging such as photoaging. Examples of dermatologic disorders include: skin disorders associated with disturbed keratinization or inflammation; wrinkles; dry skin; ichthyosis; palmar and plantar hyperkeratosis; dandruff; Darier's disease; lichen simplex chronicus; keratoses; acne; psoriasis; eczema; pruritus; keratosis pilaris, including keratosis pilaris rubra (red, inflamed bumps), alba (rough, bumpy skin with no irritation), rubra faceii (reddish rash on the cheeks); lichen planus; actinic keratosis (also called solar keratosis, or AK); seborrheic keratosis; solar lentigenes; and skin cancer, including basal cell carcinoma, squamous cell carcinoma, and melanoma.

A “disorder associated allele” or “an allele associated with a disorder” refers to an allele whose presence in a subject indicates that the subject has or is susceptible to developing a particular disorder. One type of disorder associated allele is a “dermatologic disorder associated allele,” the presence of which in a subject indicates that the subject is susceptible to aging related dermatologic disorders.

The phrases “disruption of the gene” and “targeted disruption” or any similar phrase refers to the site specific interruption of a native DNA sequence so as to prevent expression of that gene in the cell as compared to the wild-type copy of the gene. The interruption may be caused by deletions, insertions or modifications to the gene, or any combination thereof.

“Early onset of aging related dermatologic conditions” or “early onset or progression of aging related dermatologic conditions” refers to a situation wherein an aging-related dermatologic condition occurs earlier or progresses earlier than would otherwise have been expected for the particular individual and the particular condition. The expected age of onset may vary depending on the amount of information known about that individual.

The term “early progression of an aging-related condition” or “early progression of an aging-related dermatologic condition” or “EPA” is used to indicate a situation wherein the rate at which an aging related dermatologic condition progresses in a subject is more rapid than in the population as a whole. Early onset and early progression are strongly overlapping and related situations, and, unless clearly indicated by context, each of the embodiments described with respect to early onset may also be applied to early progression.

The term “haplotype” as used herein is intended to refer to a set of alleles that are inherited together as a group (are in linkage disequilibrium) at statistically significant levels (p_{corr}<0.05). As used herein, the phrase “an IL-1 haplotype” refers to a haplotype in the IL-1 loci. At least three IL-1 proinflammatory haplotypes are known. The IL-1 (44112332) (also referred to herein as pattern 2) haplotype is associated with decreased IL-receptor antagonist activity, whereas the IL-1 (33221461) (also referred to herein as pattern 1) haplotype is associated with increased IL-1α and β agonist activity. The IL-1 (44112332) haplotype includes the following alleles: IL-1RN (+2018) allele 2; IL-1RN (VNTR) allele 2; IL-1A (222/223) allele 4; IL-1A (gz5/gz6) allele 4; IL-1A (−889) allele 1; IL-1B (+3954) allele 1; IL-1B (−3737) allele 1; IL-1B (−511) allele 2; gaat.p33330 allele 3; Y31 allele 3; IL-1RN exon lic (1812) allele 2; IL-1RN exon lic (1868) allele 2; IL-1RN exon lic (1887) allele 2; Pic (1731) allele 2; IL-1A (+4845) allele 1; IL-1B (+6912) allele 1; IL-1B (−31) allele 2. The IL-1 (33221461) haplotype includes the following alleles: IL-1RN (+2018) allele 1; IL-1RN (VNTR) allele 1; IL-1A (222/223) allele 3; IL-1A (gz5/gz6) allele 3; IL-1A (−889) allele 2; IL-1B (+3954) allele 2; IL-1B (−3737) allele 1; IL-1B (−511) allele 1; gaat.p33330 allele 4; Y31 allele 6; IL-1RN exon lic (1812) allele 1; IL-1RN exon lic (1868) allele 1; IL-1RN exon lic (1887) allele 1; Pic (1731) allele 1; IL-1A (+4845) allele 2; IL-1B (+6912) allele 2; IL-1B (−31) allele 1. A third haplotype (pattern 3) comprises the following alleles: IL-1A (+4845) allele 1; IL-1A (−889) allele 1; IL-1B (+3954) allele 1; IL-1B (−511) allele 1; IL-1B (−3737) allele 2; IL-1RN (+2018) allele 1; IL-1RN (VNTR) allele 1.

An “IL-1 agonist” as used herein refers to an agent that mimics, upregulates (potentiates or supplements) or otherwise increases an IL-1 bioactivity or a bioactivity of a gene in an IL-1 biological pathway. IL-1 agonists may act on any of a variety of different levels, including regulation of IL-1 gene expression at the promoter region, regulation of mRNA splicing mechanisms, stabilization of mRNA, phosphorylation of proteins for translation, conversion of proIL-1 to mature IL-1 and secretion of IL-1. Agonists that increase IL-1 synthesis include: lipopolysaccharides, IL-1B, cAMP inducing agents, NFκKB activating agents, AP-1 activating agents, TNF-α, oxidized LDL, advanced glycosylation end products (AGE), sheer stress, hypoxia, hyperoxia, ischemia reperfusion injury, histamine, prostaglandin E 2 (PGE2), IL-2, IL-3, IL-12, granulocyte macrophage-colony stimulating factor (GM-CSF), monocyte colony stimulating factor (M-CSF), stem cell factor, platelet derived growth factor (PDGF), complement C5A, complement C5b9, fibrin degradation products, plasmin, thrombin, 9-hydroxyoctadecaenoic acid, 13-hydroxyoctadecaenoic acid, platelet activating factor (PAF), factor H, retinoic acid, uric acid, calcium pyrophosphate, polynucleosides, c-reactive protein, antitrypsin, tobacco antigen, collagen, integrins, LFA-3, anti-HLA-DR, anti-IgM, anti-CD3, CD40 ligation, phytohemagglutinin (CD2), sCD23, ultraviolet B radiation, gamma radiation, substance P, isoproterenol, methamphetamine and melatonin. Agonists that stabilize IL-1 mRNA include bacterial endotoxin and IL-1. Other agonists, that function by increasing the number of IL-1 type 1 receptors available, include IL-1, PKC activators, dexamethasone, IL-2, IL-4 and PGE2. Other preferred antagonists interfere or inhibit signal transduction factors activated by IL-1 or utilized in an IL-1 signal transduction pathway (e.g. NFκB and AP-1, P13 kinase, phospholipase A2, protein kinase C, JNK-1,5-lipoxygenase, cyclooxygenase 2, tyrosine phosphorylation, iNOS pathway, Rac, Ras, TRAF). Still other agonists increase the bioactivity of genes whose expression is induced by IL-1, including: IL-1, IL-1Ra, TNF, IL-2, IL-3, IL-6, IL-12, GM-CSF, G-CSF, TGF, fibrinogen, urokinase plasminogen inhibitor, Type 1 and type 2 plasminogen activator inhibitor, p-selectin (CD62), fibrinogen receptor, CD-11/CD18, protease nexin-1, CD44, Matrix metalloproteinase-1 (MMP-1), MMP-3, Elastase, Collagenases, Tissue inhibitor of metalloproteinases-1 (TIMP-1), Collagen, Triglyceride increasing Apo CIII, Apolipoprotein, ICAM-1, ELAM-1, VCAM-1, L-selectin, Decorin, stem cell factor, Leukemia inhibiting factor, IFNa,b,g, L-8, IL-2 receptor, IL-3 receptor, IL-5 receptor, c-kit receptor, GM-CSF receptor, Cyclooxygenase-2 (COX-2), Type 2 phospholipase A2, Inducible nitric oxide synthase (iNOS), Endothelin-1,3, Gamma glutamyl transferase, Mn superoxide dismutase, C-reactive protein, Fibrinogen, Serum amyloid A, Metallothioneins, Ceruloplasmin, Lysozyme, Xanthine dehydrogenase, Xanthine oxidase, Platelet derived growth factor A chain (PDGF), Melanoma growth stimulatory activity (gro-a,b,g), Insulin-like growth factor-i (IGF-1), Activin A, Pro-opiomelanocortiotropin, corticotropin releasing factor, B amyloid precursor, Basement membrane protein-40, Laminin B1 and B2, Constitutive heat shock protein p70, P42 mitogen, activating protein kinase, ornithine decarboxylase, heme oxygenase and G-protein a subunit).

An “IL-1 antagonist” as used herein refers to an agent that downregulates or otherwise decreases an IL-1 bioactivity. IL-1 antagonists may act on any of a variety of different levels, including, but not limited to, regulation of IL-1 gene expression at the promoter region, regulation of mRNA splicing mechanisms, stabilization of mRNA, phosphorylation of proteins for translation, conversion of proIL-1 to mature IL-1 and secretion of IL-1. Antagonists of IL-1 production include: corticosteroids, lipoxygenase inhibitors, cyclooxygenase inhibitors, gamma.-interferon, IL-4, IL-10, IL-13, transforming growth factor β (TGF-β), ACE inhibitors, n-3 polyunsaturated fatty acids, antioxidants and lipid reducing agents. Antagonists that destabilize IL-1mRNA include agents that promote deadenylation. Antagonists that inhibit or prevent phosphorylation of IL-1 proteins for translation include pyridinyl-imadazole compounds, such as tebufelone and compounds that inhibit microtubule formation (e.g. colchicine, vinblastine and vincristine). Antagonists that inhibit or prevent the conversion of proIL-1 to mature IL-1 include interleukin converting enzyme (ICE) inhibitors, CXrm-A, transcript X, endogenous tetrapeptide competitive substrate inhibitor, trypsin, elastase, chymotrypsin, chymase, and other nonspecific proteases. Antagonists that prevent or inhibit the secretion of IL-1 include agents that block anion transport. Antagonists that interfere with IL-1 receptor interactions, include: agents that inhibit glycosylation of the type I IL-1 receptor, antisense oligonucleotides against IL-1 RI, antibodies to IL-1RI and antisense oligonucleotides against IL-1RacP. Other antagonists, that function by decreasing the number of IL-1 type 1 receptors available, include TGF-α, COX inhibitors, factors that increase IL-1 type II receptors, dexamethasone, PGE2, IL-1 and IL-4. Other preferred antagonists interfere or inhibit signal transduction factors activated by IL-1 or utilized in an IL-1 signal transduction pathway (e.g NFκB and AP-1, P13 kinase, phospholipase A2, protein kinase C, JNK-1, 5-lipoxygenase, cyclooxygenase 2, tyrosine phosphorylation, iNOS pathway, Rac, Ras, TRAF). Still other antagonists interfere with the bioactivity of genes whose expression is induced by IL-1, including, but not limited to the following: IL-1, IL-1Ra, TNF, IL-2, IL-3, IL-6, IL-12, GM-CSF, G-CSF, TGF-, fibrinogen, urokinase plasminogen inhibitor, Type 1 and Type 2 plasminogen activator inhibitor, p-selectin (CD62), fibrinogen receptor, CD-11/CD18, protease nexin-1, CD44, Matrix metalloproteinase-1 (MMP-1), MMP-3, Elastase, Collagenases, Tissue inhibitor of metalloproteinases-1 (TIMP-1), Collagen, Triglyceride increasing Apo CIII, Apolipoprotein, ICAM-1, ELAM-1, VCAM-1, L-selectin, Decorin, stem cell factor, Leukemia inhibiting factor, IFN α, β, .gamma. L-8, IL-2 receptor, IL-3 receptor, IL-5 receptor, c-kit receptor, GM-CSF receptor, Cyclooxygenase-2 (COX-2), Type 2 phospholipase A2, Inducible nitric oxide synthase (iNOS), Endothelin-1,3, Gamma glutamyl transferase, Mn superoxide dismutase, C-reactive protein, Fibrinogen, Serum amyloid A, Metallothioneins, Ceruloplasmin, Lysozyme, Xanthine dehydrogenase, Xanthine oxidase, Platelet derived growth factor A chain (PDGF), Melanoma growth stimulatory activity (gro-a,b,g), Insulin-like growth factor-1 (IGF-1), Activin A, Pro-opiomelanocortiotropin, corticotropin releasing factor, B amyloid precursor, Basement membrane protein-40, Laminin B 1 and B2, Constitutive heat shock protein p70, P42 mitogen, activating protein kinase, ornithine decarboxylase, heme oxygenase and G-protein a subunit). Other preferred antagonists include: hymenialdisine, herbimycines (e.g herbamycin A), CK-103A and its derivatives (e.g. 4,6-dihydropyridazino[4,5-c]pyridazin-5 (1H)-one), CK-119, CK-122, iodomethacin, aflatoxin B1, leptin, heparin, bicyclic imidazoles (e.g SB203580), PD15306HC1, podocarpic acid derivatives, M-20, Human [Gly2] Glucagon-like peptide-2, FR167653, Steroid derivatives, glucocorticoids, Quercetin, Theophylline, NO-synthetase inhibitors, RWJ 68354, Euclyptol (1.8-cineole), Magnosalin, N-Acetylcysteine, A-Melatonin-Stimulating Hormone (a-MSH), Triclosan (2,4,4′-trichloro-2′-hydroxyldiphenyl ether), Prostaglandin E2 and 4-aminopyridine Ethacrynic acid and 4,4′-diisothiocyanatostilbene-2,2′-disulfonic acid (DIDS), Glucose, Lipophosphoglycan, aspirin, Catabolism-blocking agents, Diacerhein, Thiol-modulating agents, Zinc, Morphine, Leukotriene biosynthesis inhibitors (e.g MK886), Platelet-activating factor receptor antagonists (e.g. WEB 2086), Amiodarone, Tranilast, S-methyl-L-thiocitrulline, B-adrenoreceptor agonists (e.g Procaterol, Clenbuterol, Fenoterol, Terbutaline, Hyaluronic acid, anti-TNF-α antibodies, anti-IL-1α autoantibodies, IL-1 receptor antagonist, IL-1R-associated kinase, soluble TNF receptors and antiinflammatory cytokines (e.g. IL-4, IL-13, IL-10, IL-6, TGF-β, angiotensin II, Soluble IL-1 type II receptor, Soluble IL-1 type I receptor, Tissue plasminogen activator, Zinc finger protein A20 IL-1 Peptides (e.g. (Thr-Lys-Pro-Arg) (Tuftsin), (Ile-Thr-Gly-Ser-Glu) IL-1-α, Val-Thr-Lys-Phe-Tyr-Phe, Val-Thr-Asp-Phe-Tyr-Phe, Interferon α2b, Interferon β, IL-1-β analogues (e.g IL-1-β tripeptide: Lys-D-Pro-Thr), glycosylated IL-1-α, and IL-1ra peptides.

“IL-1 gene cluster” and “IL-1 loci” as used herein include all the nucleic acid at or near the 2q13 region of chromosome 2, including, but not limited to, at least the IL-1A, IL-1B and IL-1RN genes and any other linked sequences. The terms “IL-1A”, “IL-1B”, and “IL-1RN” as used herein refer to the genes coding for IL-1α, IL-1β, and IL-1 receptor antagonist or IL-1ra, respectively. The DNA in this region has been mapped. Nicklin et al., Genomics 19:382-84, 1994; Nothwang H. G., et al., Genomics 41:370, 1997; Clark, et al., Nucl. Acids. Res. 14:7897-914, 1986, (erratum at Nucleic Acids Res. 15:868, 1987. The gene accession numbers (GEN) for IL-1A and IL-1B, are X03833 and X04500, respectively. In general, references to nucleotide positions for IL-1RN refer to the nucleotide sequence in GEN X64532, which is the secreted form of the protein, unless there is some indication, either expressly indicated or implied from the context, that the intracellular form, which has GEN X77090, is being referenced. The two forms of IL-1RA are encoded by a single gene by alternative use of two first exons. See generally Lennard et al., Crit. Rev. Immuno. 15:77-105, 1995.

“IL-1 functional mutation” refers to a mutation within the IL-1 gene cluster that results in an altered phenotype (i.e. effects the function of an IL-1 gene or protein). Examples include: IL-1B (−511) allele 2, and IL-1RN (+2018) allele 2.

“IL-1X (Z) allele Y” refers to a particular allelic form, designated Y, occurring at an IL-1 locus polymorphic site in gene X, wherein X is IL-1A, B, or RN or some other gene in the IL-1-gene loci, and positioned at or near nucleotide Z, wherein nucleotide Z is numbered relative to the major transcriptional start site, which is nucleotide +1, of the particular IL-1 gene X. As further used herein, the term “IL-1X allele (Z)” refers to all alleles of an IL-1 polymorphic site in gene X positioned at or near nucleotide Z. For example, the term “IL-1RN (+2018) allele” refers to alternative forms of the IL-1RN gene at marker +2018. “IL-1RN (+2018) allele 1” refers to a form of the IL-1RN gene which contains a thymine (T) at position +2018 of the sense strand. Clay et al., Hum. Genet. 97:723-26, 1996. “IL-1RN (+2018) allele 2” refers to a form of the IL-1RN gene which contains a cytosine (C) at position +2018 of the plus strand. When a subject has two identical IL-1RN alleles, the subject is said to be homozygous, or to have the homozygous state. When a subject has two different IL-1RN alleles, the subject is said to be heterozygous, or to have the heterozygous state. The term “IL-1RN (+2018) allele 2,2” refers to the homozygous IL-1RN (+2018) allele 2 state. Conversely, the term “IL-1RN (+2018) allele 1,1” refers to the homozygous IL-1RN (+2018) allele 1 state. The term “IL-1RN (+2018) allele 1,2” refers to the heterozygous allele 1 and 2 state.

“IL-1 related” as used herein is meant to include all genes related to the human IL-1 locus genes on human chromosome 2 (2q 12-14). These include IL-1 genes of the human IL-1 gene cluster located at chromosome 2 (2q 13-14) which include: the IL-1A gene which encodes interleukin-1a, the IL-1B gene which encodes interleukin-1β, and the IL-1RN (or IL-1ra) gene which encodes the interleukin-1 receptor antagonist. Furthermore these IL-1 related genes include the type I and type II human IL-1 receptor genes located on human chromosome 2 (2q12) and their mouse homologs located on mouse chromosome 1 at position 19.5 cM. Interleukin-1, interleukin-1, and interleukin-1RN are related in so much as they all bind to IL-1 type I receptors, however only interleukin-1 and interleukin-1 are agonist ligands which activate IL-1 type I receptors, while interleukin-1RN is a naturally occurring antagonist ligand. Where the term “IL-1” is used in reference to a gene product or polypeptide, it is meant to refer to all gene products encoded by the interleukin-1 locus on human chromosome 2 (2q 12-14) and their corresponding homologs from other species or functional variants thereof. The term IL-1 thus includes secreted polypeptides which promote an inflammatory response, such as IL-1 and IL-1β, as well as a secreted polypeptide which antagonize inflammatory responses, such as IL-1α receptor antagonist and the IL-1 type II (decoy) receptor.

An “IL-1 receptor” or “IL-1R” refers to various cell membrane bound protein receptors capable of binding to and/or transducing a signal from IL-1 locus-encoded ligand. The term applies to any of the proteins which are capable of binding interleukin-1 (IL-1) molecules and, in their native configuration as mammalian plasma membrane proteins, presumably play a role in transducing the signal provided by IL-1 to a cell. As used herein, the term includes analogs of native proteins with IL-1-binding or signal transducing activity. Examples include the human and murine IL-1 receptors described in U.S. Pat. No. 4,968,607. The term “IL-1 nucleic acid” refers to a nucleic acid encoding an IL-1 protein.

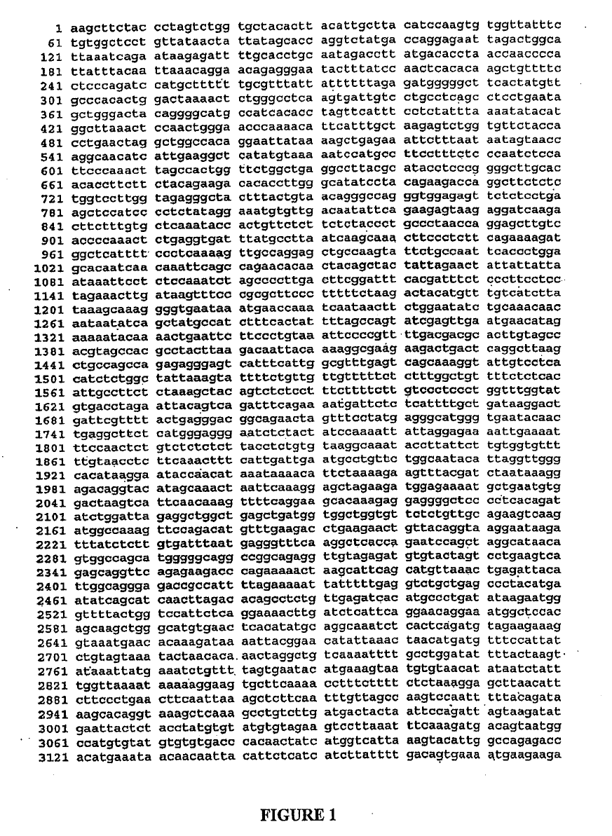

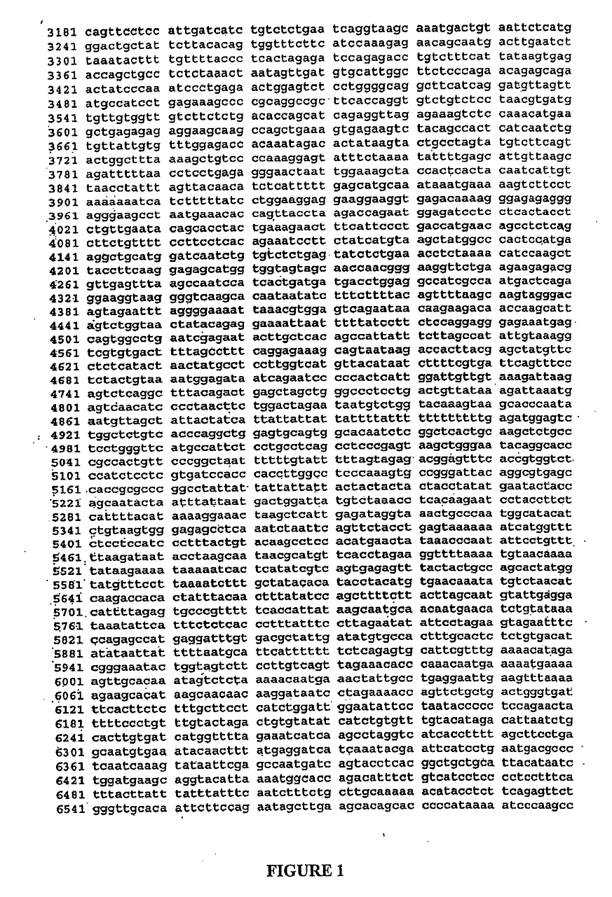

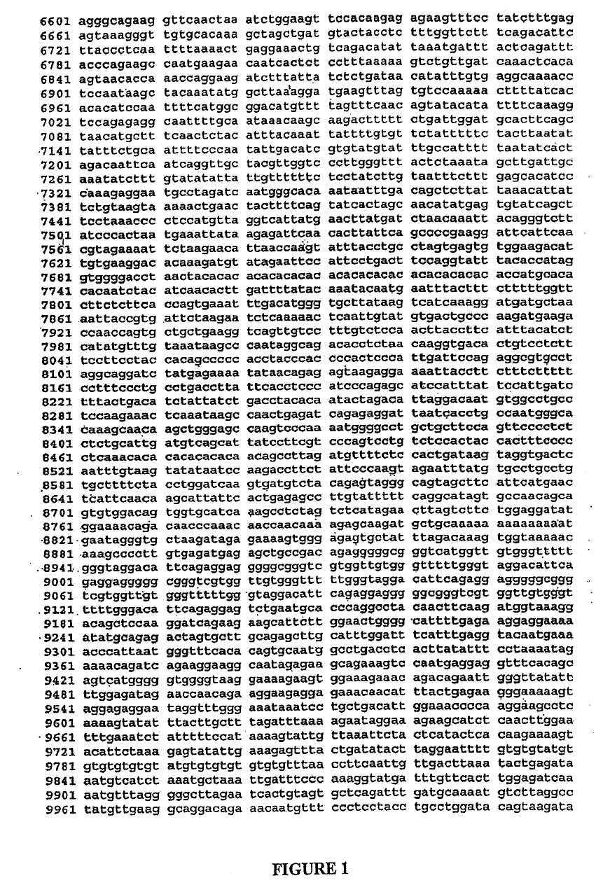

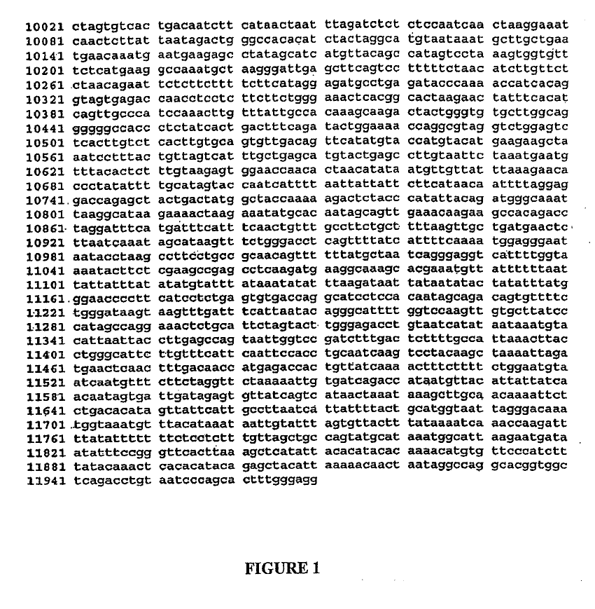

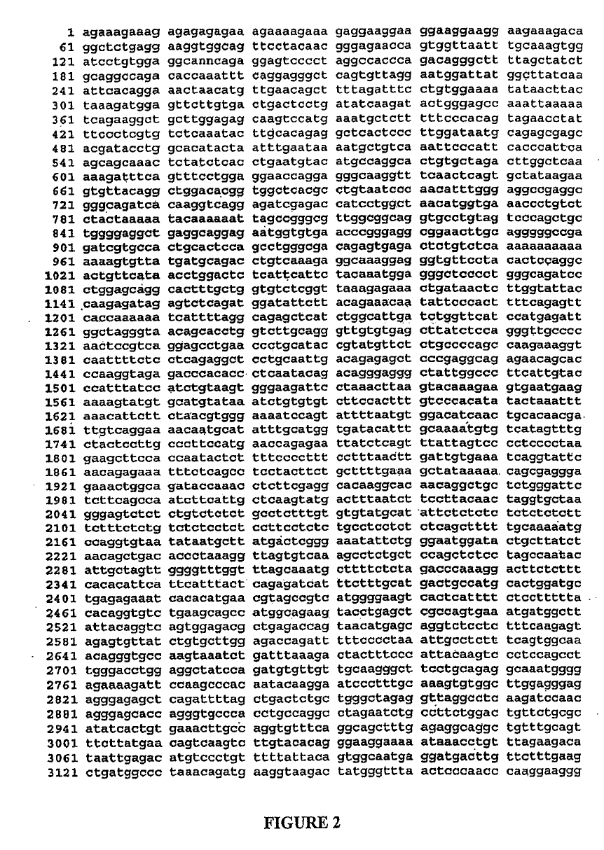

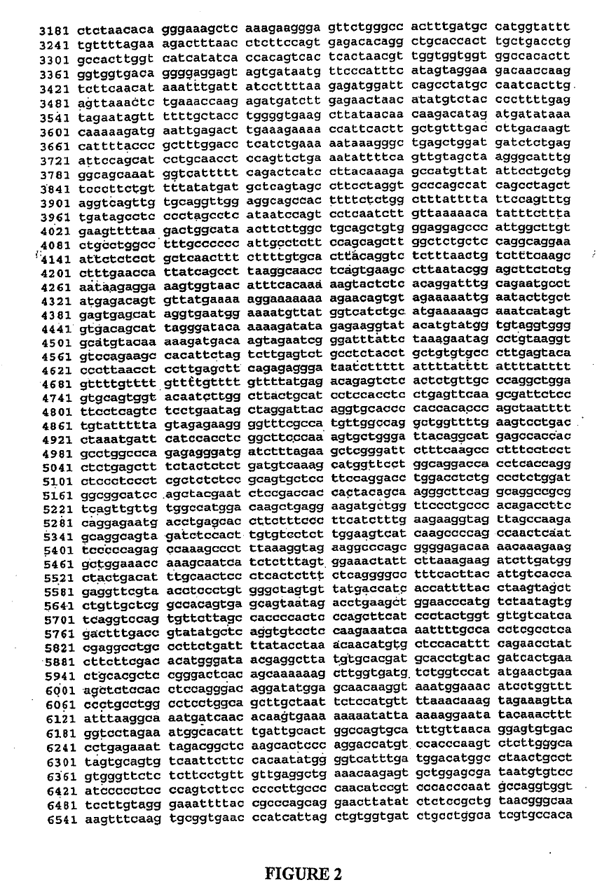

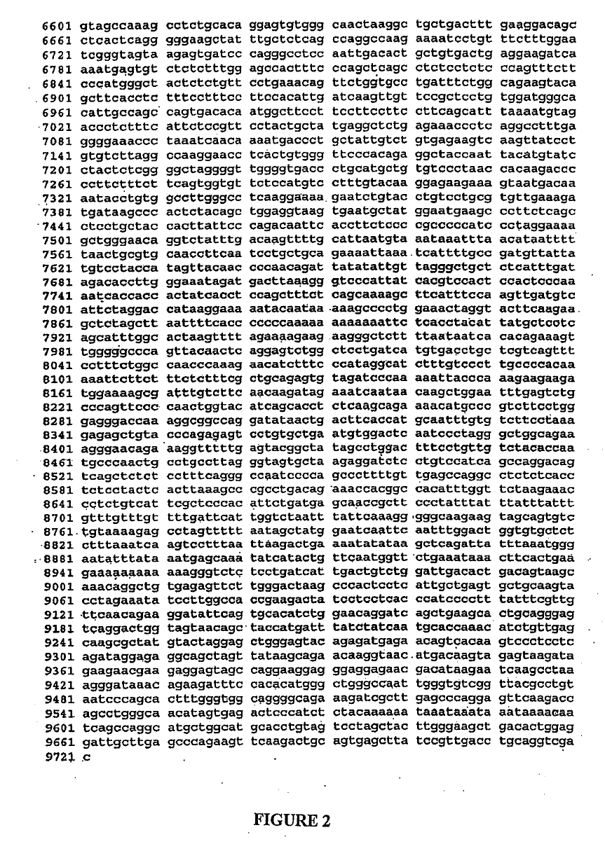

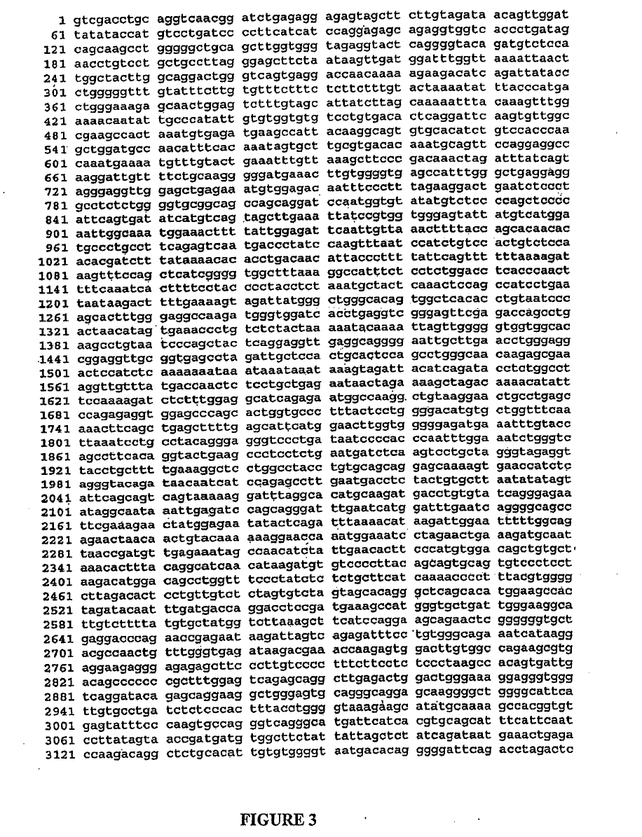

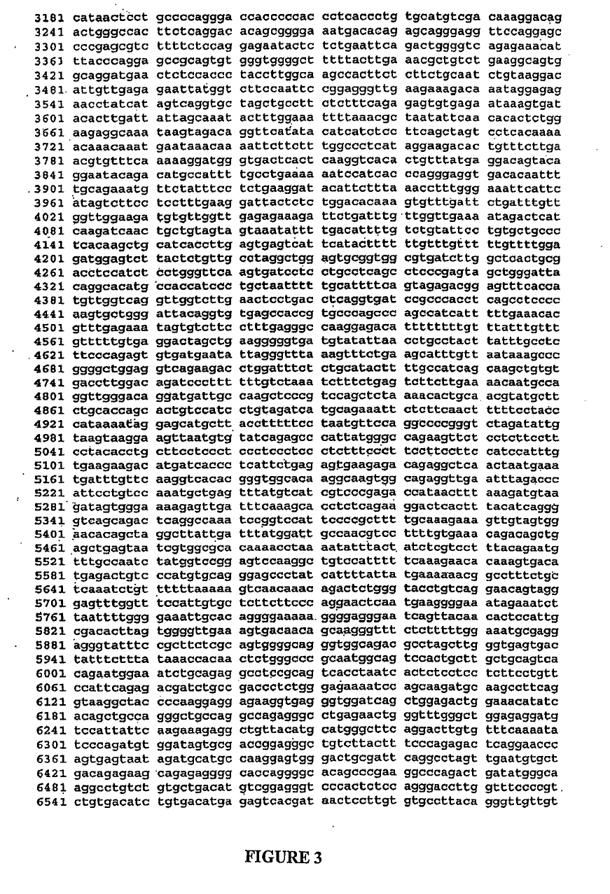

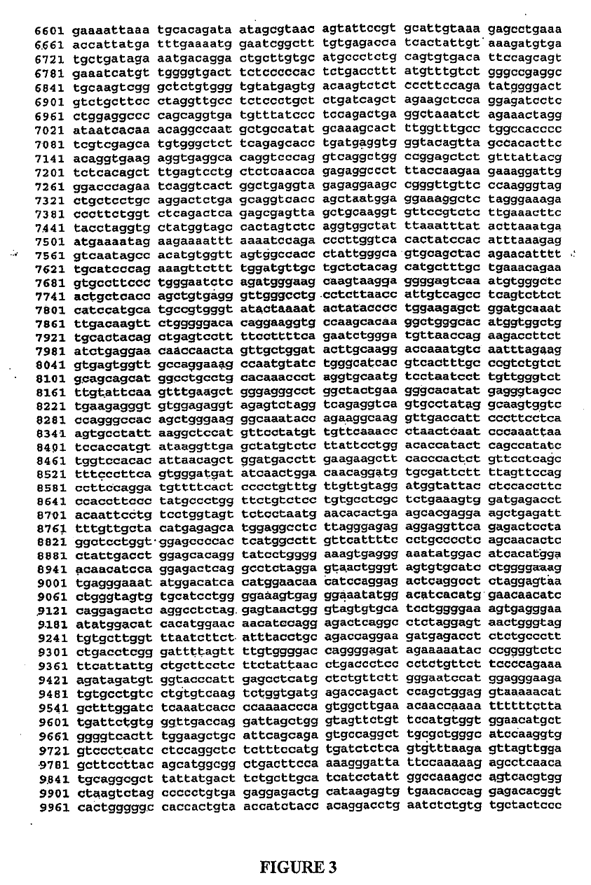

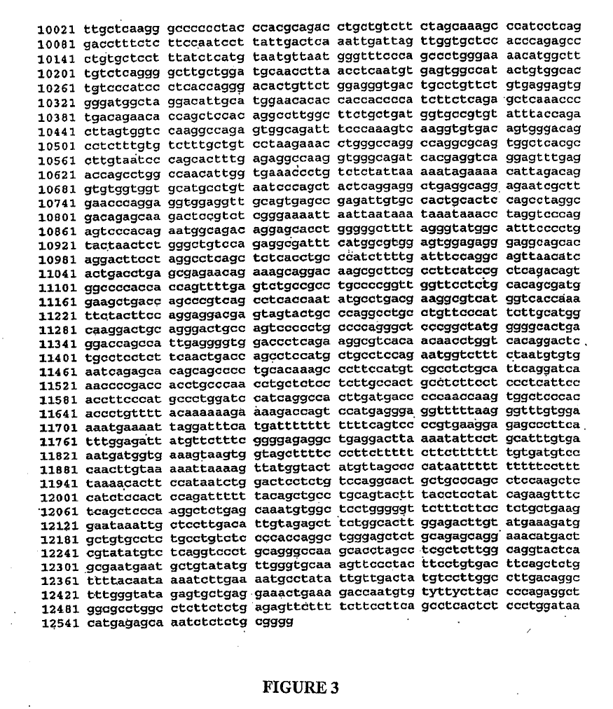

An “IL-1 polypeptide” and “IL-1 protein” are intended to encompass polypeptides comprising the amino acid sequence encoded by the IL-1 genomic DNA sequences shown in FIGS. 1, 2, and 3, or fragments thereof, and homologs thereof and include agonist and antagonist polypeptides.

The “immune system” is a complex system of cells and factors that functions to prevent infection by viruses, bacteria, parasites, helminths, fungi, insects, protozoans etc, and to protect against foreign bodies or non-self material generally. The immune system also functions to destroy damaged or diseased cells of the body, including, but not limited to, cancer cells. The immune system further functions to discriminate between self and non-self, and mediates inflammation and systemic shock. Impaired immune system function refers to defects in any of these activities.

The term “including” is used herein to mean “including but not limited to”. “Including” and “including but not limited to” are used interchangeably.

“Increased risk” or “increased susceptibility” refers to a statistically higher frequency of occurrence of the disease or condition in an individual carrying a particular polymorphic allele in comparison to the frequency of occurrence of the disease or condition in a member of a population that does not carry the particular polymorphic allele.

The term “interact” as used herein is meant to include detectable relationships or associations (e.g. biochemical interactions) between molecules, such as interactions between protein-protein, protein-nucleic acid, nucleic acid-nucleic acid and protein-small molecule or nucleic acid-small molecule in nature.

The term “isolated” as used herein with respect to nucleic acids, such as DNA or RNA, refers to molecules separated from other DNAs, or RNAs, respectively, that are present in the natural source of the macromolecule. For example, an isolated nucleic acid encoding one of the subject IL-1 polypeptides preferably includes no more than 10 kilobases (kb) of nucleic acid sequence which naturally immediately flanks the IL-1 gene in genomic DNA, more preferably no more than 5 kb of such naturally occurring flanking sequences, and most preferably less than 1.5 kb of such naturally occurring flanking sequence. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides.

A “knock-in” transgenic animal refers to an animal that has had a modified gene introduced into its genome and the modified gene can be of exogenous or endogenous origin.

A “knock-out” transgenic animal refers to an animal in which there is partial or complete suppression of the expression of an endogenous gene (e.g, based on deletion of at least a portion of the gene, replacement of at least a portion of the gene with a second sequence, introduction of stop codons, the mutation of bases encoding critical amino acids, or the removal of an intron junction, etc.).

A “knock-out construct” refers to a nucleic acid sequence that can be used to decrease or suppress expression of a protein encoded by endogenous DNA sequences in a cell. In a simple example, the knock-out construct is comprised of a gene, such as the IL-1RN gene, with a deletion in a critical portion of the gene so that active protein cannot be expressed therefrom. Alternatively, a number of termination codons can be added to the native gene to cause early termination of the protein or an intron junction can be inactivated. In a typical knock-out construct, some portion of the gene is replaced with a selectable marker (such as the neo gene) so that the gene can be represented as follows: IL-1RN 5′/neo/IL-1RN 3′, where IL-1RN5′ and IL-1 RN 3′, refer to genomic or cDNA sequences which are, respectively, upstream and downstream relative to a portion of the IL-1RN gene and where neo refers to a neomycin resistance gene. In another knock-out construct, a second selectable marker is added in a flanking position so that the gene can be represented as: IL-1RN/neo/IL-1RN/TK, where TK is a thymidine kinase gene which can be added to either the IL-1 RN5′ or the IL-1 RN3′ sequence of the preceding construct and which further can be selected against (i.e. is a negative selectable marker) in appropriate media. This two-marker construct allows the selection of homologous recombination events, which removes the flanking TK marker, from non-homologous recombination events which typically retain the TK sequences. The gene deletion and/or replacement can be from the exons, introns, especially intron junctions, and/or the regulatory regions such as promoters.

“Linkage disequilibrium” refers to co-inheritance of two alleles at frequencies greater than would be expected from the separate frequencies of occurrence of each allele in a given control population. The expected frequency of occurrence of two alleles that are inherited independently is the frequency of the first allele multiplied by the frequency of the second allele. Alleles that co-occur at expected frequencies are said to be in “linkage equilibrium”. The cause of linkage disequilibrium is often unclear. It can be due to selection for certain allele combinations or to recent admixture of genetically heterogeneous populations. In addition, in the case of markers that are very tightly linked to a disease gene, an association of an allele (or group of linked alleles) with the disease gene is expected if the disease mutation occurred in the recent past, so that sufficient time has not elapsed for equilibrium to be achieved through recombination events in the specific chromosomal region. When referring to allelic patterns that are comprised of more than one allele, a first allelic pattern is in linkage disequilibrium with a second allelic pattern if all the alleles that comprise the first allelic pattern are in linkage disequilibrium with at least one of the alleles of the second allelic pattern. An example of linkage disequilibrium is that which occurs between the alleles at the IL-1RN (+2018) and IL-1RN (VNTR) polymorphic sites. The two alleles at IL-1RN (+2018) are 100% in linkage disequilibrium with the two most frequent alleles of IL-1RN (VNTR), which are allele 1 and allele 2. Examples of linked polymorphic markers in linkage disequilibrium with IL-1B (−511) include: the 222/223 marker of IL-1A, the gz5/gz6 marker of IL-1A, the −889 marker of IL-1A, the +6912 marker of L-1B, the +3953 marker of IL-1B, the gaat.p33330 marker of the IL-1B/IL-1RN intergenic region, the Y31 marker of the IL-1B/IL-1RN intergenic region, the +2018 allele of the IL-1RN, or the VNTR marker of IL-1RN. Specific alleles of these polymorphic markers are in linkage disequilibrium with allele 1 or allele 2 of IL-1B (−511). For example, linkage disequilibrium analysis between pair-wise combinations of these alleles has established that allele 2 of IL-1B (−511) is in linkage disequilibrium with: allele 4 of IL-1A 222/223, allele 4 of IL-1A gz5/gz6, allele 1 of IL-1A -889, allele 1 of IL-1A +3953, allele 3 of the gaat.p3330 marker, allele 3 of the Y31 marker, allele 2 of IL-1B +2018, and allele 2 of the IL-1RN VNTR. Examples of other linked polymorphisms include four polymorphisms in the IL-1RN gene (Clay et al. (1996) Hum. Genet. 97: 723-26). Linkage disequilibrium analysis of these polymorphisms indicates that allele 2 of each is in linkage disequilibrium with allele 2 of IL-1B (−511).

The term “marker” refers to a sequence in the genome that is known to vary among individuals. For example, the IL-1RN gene has a marker that consists of a variable number of tandem repeats (VNTR). The different sequence variants at a given marker are called alleles, mutations or polymorphisms. For example, the VNTR marker has at least five different alleles, three of which are rare. Different alleles could have a single base change, including substitution, insertion or deletion, or could have a change that affects multiple bases, including substitutions, insertions, deletions, repeats, inversions and combinations thereof.

“Modulate” refers to the ability of a substance to regulate bioactivity. When applied to an IL-1 bioactivity, an agonist or antagonist can modulate bioactivity for example by agonizing or antagonizing an IL-1 synthesis, receptor interaction, or IL-1 mediated signal transduction mechanism.

A “mutated gene” or “mutation” or “functional mutation” refers to an allelic form of a gene, which is capable of altering the phenotype of a subject having the mutated gene relative to a subject which does not have the mutated gene. The altered phenotype caused by a mutation can be corrected or compensated for by certain agents. If a subject must be homozygous for this mutation to have an altered phenotype, the mutation is said to be recessive. If one copy of the mutated gene is sufficient to alter the phenotype of the subject, the mutation is said to be dominant. If a subject has one copy of the mutated gene and has a phenotype that is intermediate between that of a homozygous and that of a heterozygous subject (for that gene), the mutation is said to be co-dominant.

A “non-human animal” of the invention includes mammals such as rodents, non-human primates, sheep, dogs, cows, goats, etc. Preferred non-human animals are selected from the rodent family including rat and mouse, most preferably mouse, though transgenic amphibians, such as members of the Xenopus genus, and transgenic chickens can also provide important tools for understanding and identifying agents which can affect, for example, embryogenesis and tissue formation. The term “chimeric animal” is used herein to refer to animals in which the recombinant gene is found, or in which the recombinant gene is expressed in some but not all cells of the animal. The term “tissue-specific chimeric animal” indicates that one of the recombinant IL-1 genes is present and/or expressed or disrupted in some tissues but not others. The term “non-human mammal” refers to any members of the class Mammalia, except for humans.

As used herein, the term “nucleic acid” refers to polynucleotides or oligonucleotides such as deoxyribonucleic acid (DNA), and, where appropriate, ribonucleic acid (RNA). The term should also be understood to include, as equivalents, analogs of either RNA or DNA made from nucleotide analogs (e.g. peptide nucleic acids) and as applicable to the embodiment being described, single (sense or antisense) and double-stranded polynucleotides.

“Nutraceuticals” are defined as substances comprising vitamins, minerals, proteins, amino acids, sugars, phytoestrogens, flavonoids, phenolics, anthocyanins, carotenoids, polymers of the above, and mixtures of the above.

The term “or” as used herein should be understood to mean “and/or”, unless the context clearly indicates otherwise.

The term “polymorphism” refers to the coexistence of more than one form of a gene or portion (e.g., allelic variant) thereof. A portion of a gene of which there are at least two different forms, i.e., two different nucleotide sequences, is referred to as a “polymorphic region.” As used herein, the term “polymorphic region” includes, without limitation, a polymorphic site consisting of a single nucleotide, e.g., a single nucleotide polymorphism (SNP). A specific genetic sequence at a polymorphic region is an allele. A polymorphic region can be a single nucleotide, the identity of which differs in different alleles. A polymorphic region can also be more than one nucleotide long, and possibly significantly longer in length.

The term “propensity” as used herein in reference to a condition or disease state, as in “propensity” for a condition or disease, is used interchangeably with the expressions “susceptibility” or “predisposition”. The term “propensity” as used in reference to a condition or disease state indicates that an individual is at increased risk for the future development of a condition or disease. For example, if an allele is discovered to be associated with or predictive of a particular disease or condition, an individual carrying the allele has a greater propensity for developing the particular disease or condition.

“Small molecule” as used herein, is meant to refer to a composition, which has a molecular weight of less than about 5 kD and most preferably less than about 4 kD. Small molecules can be nucleic acids, peptides, peptidomimetics, carbohydrates, lipids or other organic or inorganic molecules.

As used herein, the term “specifically hybridizes” or “specifically detects” refers to the ability of a nucleic acid molecule to hybridize to at least approximately 6 consecutive nucleotides of a sample nucleic acid.

“Transcriptional regulatory sequence” is a generic term used throughout the specification to refer to DNA sequences, such as initiation signals, enhancers, and promoters, which induce or control transcription of protein coding sequences with which they are operably linked.

As used herein, the term “transgene” means a nucleic acid sequence (encoding, e.g., one of the IL-1 polypeptides, or an antisense transcript thereto) which has been introduced into a cell. A transgene could be partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can also be present in a cell in the form of an episome. A transgene can include one or more transcriptional regulatory sequences and any other nucleic acid, such as introns, that may be necessary for optimal expression of a selected nucleic acid.

A “transgenic animal” refers to any animal, preferably a non-human mammal, bird or an amphibian, in which one or more of the cells of the animal contain heterologous nucleic acid introduced by way of human intervention, such as by transgenic techniques well known in the art. The nucleic acid is introduced into the cell, directly or indirectly by introduction into a precursor of the cell, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extrachromosomally replicating DNA. In the typical transgenic animals described herein, the transgene causes cells to express a recombinant form of one of an IL-1 polypeptide, e.g. either agonistic or antagonistic forms. However, transgenic animals in which the recombinant gene is silent are also contemplated, as for example, the FLP or CRE recombinase dependent constructs described below. Moreover, “transgenic animal” also includes those recombinant animals in which gene disruption of one or more genes is caused by human intervention, including both recombination and antisense techniques. The term is intended to include all progeny generations. Thus, the founder animal and all F1, F2, F3, and so on, progeny thereof are included.

The term “treating” as used herein is intended to encompass curing as well as ameliorating at least one symptom of a disease or at least one abnormality associated with a disorder.

The term “vector” refers to a nucleic acid molecule, which is capable of transporting another nucleic acid to which it has been linked. One type of preferred vector is an episome, i.e., a nucleic acid capable of extra-chromosomal replication. Preferred vectors are those capable of autonomous replication and/or expression of nucleic acids to which they are linked. Vectors capable of directing the expression of genes to which they are operatively linked are referred to herein as “expression vectors”. In general, expression vectors of utility in recombinant DNA techniques are often in the form of “plasmids” which refer generally to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The term “wild-type allele” refers to an allele of a gene which, when present in two copies in a subject results in a wild-type phenotype. There can be several different wild-type alleles of a specific gene, since certain nucleotide changes in a gene may not affect the phenotype of a subject having two copies of the gene with the nucleotide changes.

Predictive Medicine

Polymorphisms Associated with Genetic Predisposition To Hyper-Inflammatory Response

The present invention is based at least in part, on the identification of alleles that are associated with the development of aging-related dermatologic conditions. Therefore, detection of these alleles, alone or in conjunction with another means in a subject indicate that the subject has or is predisposed to aging-related dermatologic conditions. For example, IL-1 polymorphic alleles which are associated with aging-related dermatologic conditions include allele 2 of each of the following markers: IL-1B (−511) allele 2 and IL1RN (+2018) allele 2 or an allele that is in linkage disequilibrium with one of the aforementioned alleles. Thus, detection of IL-1B (−511) allele 2 and IL1RN (+2018) allele 2 indicates that a subject has a reduced predisposition for developing early onset or progression of aging related dermatologic conditions.

In certain embodiments, the presence of a particular allelic pattern of one or more of the above mentioned IL-1 polymorphic loci may be used to predict the susceptibility of an individual to developing aging-related dermatologic conditions. In particular, there are three patterns of alleles at loci in the IL-1 gene cluster that show various associations with aging-related dermatologic conditions. These patterns are referred to herein as patterns 1, 2 and 3.

Pattern 1 includes IL-1A (+4845) allele 2, IL-1B (+3954) allele 2, IL-1B (−511) allele 1, IL-1B (−3737) allele 1, and IL-1RN (+2018) allele 1. Pattern 1 may comprise IL-1A (+4845) allele 2 (homozygous/heterozygous), IL-1B (+3954) allele 2 (homozygous/heterozygous), IL-1B (−511) allele 1 (homozygous), IL-1B (−3737) allele 1 (homozygous/heterozygous), and IL-1RN (+2018) allele 1 (homozygous).

Pattern 2 includes IL-1A (+4845) allele 1, IL-1B (+3954) allele 1, IL-1B (−511) allele 2, IL-1B (−3737) allele 1, and IL-1RN (+2018) allele 2. Pattern 2 may comprise IL-1A (+4845) allele 1 (homozygous), IL-1B (+3954) allele 1 (homozygous), IL-1B (−511) allele 2 (homozygous), IL-1B (−3737) allele 1 (homozygous), and IL-1RN (+2018) allele 2 (homozygous/heterozygous).

Pattern 3 includes IL-1A (+4845) allele 1, IL-1B (+3954) allele 1, IL-1B (−511) allele 1, IL-1B (−3737) allele 2, and IL-1RN (+2018) allele 1. Pattern 3 may comprise IL-1A (+4845) allele 1 (homozygous), IL-1B (+3954) allele 1 (homozygous), IL-1B (−511) allele 1 (homozygous), IL-1B (−3737) allele 2 (homozygous), and IL-1RN (+2018) allele 1 (homozygous).

In a preferred embodiment, this detection of any of these patterns provides information about the likelihood that the subject will develop an aging-related dermatologic conditions. Detection of pattern 2 indicates that a subject has a reduced predisposition for developing early onset or progression of aging related dermatologic conditions. Patterns 1 and 3 indicates that a subject has an increased predisposition for developing early onset or progression of aging related dermatologic conditions.

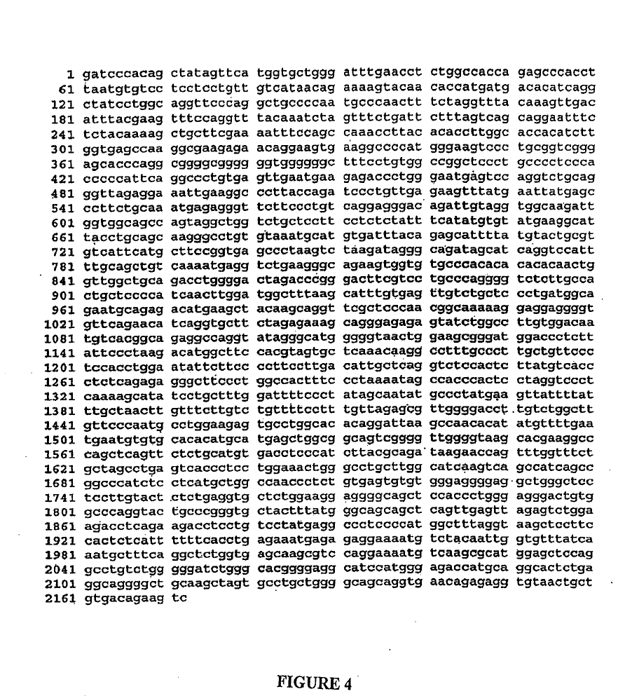

The IL-1 locus polymorphisms represent single base variations within the IL-1A/IL-1B/IL-1RN gene cluster (see FIG. 4). The IL-1A (+4845) polymorphism is a single base variation (allele 1 is G, allele 2 is T) at position +4845 within Exon V of the IL-1A gene which encodes the inflammatory cytokine IL-1α (Gubler, et al. (1989) Interleukin, inflammation and disease (Bbmford and Henderson, eds.) p. 31-45, Elsevier publishers; and Van den velden and Reitsma (1993) Hum Mol Genetics 2:1753-50). The IL-1A (+4845) polymorphism occurs in the coding region of the gene and results in a single amino acid variation in the encoded protein (Van den Velden and Reitsma (1993) Hum Mol Genet. 2: 1753).

The IL-1B (+3954) polymorphism was first described as a Taq I restriction fragment length polymorphism (RFLP) (Pociot et al. (1992) Eur J Clin Invest 22: 396-402) and has subsequently been characterized as a single base variation (allele 1 is C, allele 2 is T) at position +3954 in Exon V of the IL-1B gene (di Giovine et al. (1995) Cytokine 7: 600-606). This single nucleotide change in the open reading frame of IL-1B does not appear to qualitatively affect the sequence of the encoded IL-1β polypeptide because it occurs at the third position of a TTC phenylalanine codon (F) of allele 1 and therefore allele 2 merely substitutes a TTT phenylalanine codon at this position which encodes amino acid 105 of the IL-1B gene product.

In addition, the IL-1RN (+2018) polymorphism (Clay et al. (1996) Hum Genet. 97: 723-26) is a single base variation (allele 1 is T, allele 2 is C), also referred to as exon 2 (8006) (GenBank: X64532 at 8006). Finally, the IL-RN variable number of tandem repeats (VNTR) polymorphism occurs within the second intron the IL-1 receptor antagonist encoding gene (Steinkasserer (1991) Nucleic Acids Res 19: 5090-5). Allele 2 of the of the IL-1RN (VNTR) polymorphism corresponds to two repeats of an 86-base pair sequence, while allele 1 corresponds to four repeats, allele 3 to three repeats, allele 4 to five repeats, and allele 5 to six repeats (Tarlow et al. (1993) Hum Genet. 91: 403-4). Also, allele 2 of the IL-1RN (+2018) polymorphism is in strong linkage disequilibrium with allele 2 of the IL-1RN (VNTR) polymorphism. (Duff et al. U.S. Pat. No. 6,746,839, incorporated by reference, herein, in its entirety).

Two bi-allelic polymorphisms can be typed in two different PCR products using allele-specific cleavage at naturally-occurring sites in the alleles. Allele identification is by size of fragment after restriction digestion and separation in an agarose gel. The gene is designated IL-1B while the product (cytokine) is designated IL-1β. The sites are single base variations (C/T) at −511 (referred to as IL-1B (AvaI)) and at +3953 (referred to as IL-1B (TaqI)) and are identified by allele-specific cleavage using restriction enzymes. For each polymorphism allele 1 is C and allele 2 is T.

The term IL-1B (−511) allele 2 describes allele 2 of the −511 marker of the IL-1B gene. This allele contains a Bsu361 site and produces 190 and 114 bp fragments when amplified with the primers described herein and digested with Bsu361. de Giovine et al., “Single base polymorphism at −511 in the human interleukin-1.β. gene (IL1.β.)” Human Molecular Genetics 1, No. 6:450 (1992).

In addition to the allelic patterns described above, one of skill in the art can, in view of this specification, readily identify other alleles (including polymorphisms and mutations) that are in linkage disequilibrium with an allele associated with aging-related dermatologic disorder. For example, a nucleic acid sample from a first group of subjects without known aging-related dermatologic disorder associated alleles can be collected, as well as DNA from a second group of subjects carrying one or more aging-related dermatologic disorder associated alleles. The nucleic acid sample can then be compared to identify those alleles that are over-represented in the second group as compared with the first group, wherein such alleles are presumably associated with aging-related dermatologic disorder. Alternatively, alleles that are in linkage disequilibrium with an aging-related dermatologic disorder associated allele can be identified, for example, by genotyping a large population and performing statistical analysis to determine which alleles appear more commonly together than expected. Preferably the group is chosen to be comprised of genetically related individuals. Genetically related individuals include individuals from the same race, the same ethnic group, or even the same family. As the degree of genetic relatedness between a control group and a test group increases, so does the predictive value of polymorphic alleles which are ever more distantly linked to a disease-causing allele. This is because less evolutionary time has passed to allow polymorphisms which are linked along a chromosome in a founder population to redistribute through genetic cross-over events. Thus race-specific, ethnic-specific, and even family-specific diagnostic genotyping assays can be developed to allow for the detection of disease alleles which arose at ever more recent times in human evolution, e.g., after divergence of the major human races, after the separation of human populations into distinct ethnic groups, and even within the recent history of a particular family line.

Linkage disequilibrium between two polymorphic markers or between one polymorphic marker and a disease-causing mutation is a meta-stable state. Absent selective pressure or the sporadic linked reoccurrence of the underlying mutational events, the polymorphisms will eventually become disassociated by chromosomal recombination events and will thereby reach linkage equilibrium through the course of human evolution. Thus, the likelihood of finding a polymorphic allele in linkage disequilibrium with a disease or condition may increase with changes in at least two factors: decreasing physical distance between the polymorphic marker and the disease-causing mutation, and decreasing number of meiotic generations available for the dissociation of the linked pair. Consideration of the latter factor suggests that, the more closely related two individuals are, the more likely they will share a common parental chromosome or chromosomal region containing the linked polymorphisms and the less likely that this linked pair will have become unlinked through meiotic cross-over events occurring each generation. As a result, the more closely related two individuals are, the more likely it is that widely spaced polymorphisms may be co-inherited. Thus, for individuals related by common race, ethnicity or family, the reliability of ever more distantly spaced polymorphic loci can be relied upon as an indicator of inheritance of a linked disease-causing mutation.

Examples of linked polymorphic markers in linkage disequilibrium with IL-1B (−511) include: the 222/223 marker of IL-1A, the gz5/gz6 marker of IL-1A, the −889 marker of IL-1A, the +6912 marker of L-1B, the +3953 marker of IL-1B, the gaat.p33330 marker of the IL-1B/IL-1RN intergenic region, the Y31 marker of the IL-1B/IL-1RN intergenic region, the +2018 allele of the IL-1RN , or the VNTR marker of IL-1RN . Specific alleles of these polymorphic markers are in linkage disequilibrium with allele 1 or allele 2 of IL-1B (−511). For example, linkage disequilibrium analysis between pair-wise combinations of these alleles has established that allele 2 of IL-1B (−511) is in linkage disequilibrium with: allele 4 of IL-1A 222/223, allele 4 of IL-1A gz5/gz6, allele 1 of IL-1A -889, allele 1 of IL-1A +3953, allele 3 of the gaat.p3330 marker, allele 3 of the Y31 marker, allele 2 of IL-1B+2018, and allele 2 of the IL-1RN VNTR. Examples of other linked polymorphisms include four polymorphisms in the IL-1RN gene (Clay et al. (1996) Hum. Genet. 97: 723-26). Linkage disequilibrium analysis of these polymorphisms indicates that allele 2 of each is in linkage disequilibrium with allele 2 of IL-1B (−511).

Appropriate probes may be designed to hybridize to a specific gene of the IL-1 locus, such as IL-1A, IL-1B or IL-1RN or a related gene. These genomic DNA sequences are shown in FIGS. 1, 2 and 3, respectively, and further correspond to formal SEQ ID Nos. 15, 16 and 17, respectively. Alternatively, these probes may incorporate at other regions of the relevant genomic locus, including intergenic sequences. Indeed the IL-1 region of human chromosome 2 spans some 400,000 base pairs and, assuming an average of one single nucleotide polymorphism every 1,000 base pairs, includes some 400 SNPs loci alone. Yet other polymorphisms available for use with the immediate invention are obtainable from various public sources. For example, the human genome database collects intragenic SNPs, is searchable by sequence and currently contains approximately 2,700 entries. Also available is a human polymorphism database maintained by the Massachusetts Institute of Technology (MIT SNP database). From such sources SNPs as well as other human polymorphisms may be found.

For example, examination of the IL-1 region of the human genome in any one of these databases reveals that the IL-1 locus genes are flanked by a centromere proximal polymorphic marker designated microsatellite marker AFM220ze3 at 127.4 cM (centiMorgans) (see GenBank Acc. No. Z17008) and a distal polymorphic marker designated microsatellite anchor marker AFMO87xa1 at 127.9 cM (see GenBank Acc. No. Z16545). These human polymorphic loci are both CA dinucleotide repeat microsatellite polymorphisms, and, as such, show a high degree of heterozygosity in human populations. For example, one allele of AFM220ze3 generates a 211 bp PCR amplification product with a 5′ primer of the sequence TGTACCTAAGCCCACCCTTTAGAGC (SEQ ID No. 18) and a 3′ primer of the sequence TGGCCTCCAGAAACCTCCAA (SEQ ID No. 19). Furthermore, one allele of AFM087xa1 generates a 177 bp PCR amplification product with a 5′ primer of the sequence GCTGATATTCTGGTGGGAAA (SEQ ID No. 20) and a 3′ primer of the sequence GGCAAGAGCAAAACTCTGTC (SEQ ID No. 21). Equivalent primers corresponding to unique sequences occurring 5′ and 3′ to these human chromosome 2 CA dinucleotide repeat polymorphisms will be apparent to one of skill in the art. Reasonable equivalent primers include those which hybridize within about 1 kb of the designated primer, and which further are anywhere from about 17 bp to about 27 bp in length. A general guideline for designing primers for amplification of unique human chromosomal genomic sequences is that they possess a melting temperature of at least about 50 C, wherein an approximate melting temperature can be estimated using the formula Tₘₑₗₜ=[2×(# of A or T)+4×(# of G or C)].

A number of other human polymorphic loci occur between these two CA dinucleotide repeat polymorphisms and provide additional targets for determination of a ARDD prognostic allele in a family or other group of genetically related individuals. For example, the National Center for Biotechnology Information web site lists a number of polymorphism markers in the region of the IL-1 locus and provides guidance in designing appropriate primers for amplification and analysis of these markers.

Accordingly, the nucleotide segments of the invention may be used for their ability to selectively form duplex molecules with complementary stretches of human chromosome 2 q 12-13 or cDNAs from that region or to provide primers for amplification of DNA or cDNA from this region. The design of appropriate probes for this purpose requires consideration of a number of factors. For example, fragments having a length of between 10, 15, or 18 nucleotides to about 20, or to about 30 nucleotides, will find particular utility. Longer sequences, e.g., 40, 50, 80, 90, 100, even up to full length, are even more preferred for certain embodiments. Lengths of oligonucleotides of at least about 18 to 20 nucleotides are well accepted by those of skill in the art as sufficient to allow sufficiently specific hybridization so as to be useful as a molecular probe. Furthermore, depending on the application envisioned, one will desire to employ varying conditions of hybridization to achieve varying degrees of selectivity of probe towards target sequence. For applications requiring high selectivity, one will typically desire to employ relatively stringent conditions to form the hybrids. For example, relatively low salt and/or high temperature conditions, such as provided by 0.02 M-0.15 M NaCl at temperatures of about 50 C to about 70 C. Such selective conditions may tolerate little, if any, mismatch between the probe and the template or target strand.

Other alleles or other indicia of aging-related dermatologic disorders may be detected or monitored in a subject in conjunction with detection of the alleles described above.

List of SNPs

| | |
|---|---|
| IL1 A (+4845) | rs17561 G > T |
| IL1 [symbol] (#31 511) | rs16944 C > T |
| IL1 B (−3737) | rs4848306 C > T |
| IL1 B (+3954) | rs1143634 C > T |
| IL1 [symbol] (#30 3877) | rs1143633 G > A |
| IL1RN (+2018) | rs419598 T > C |

Detection of Alleles/Haplotype Determination

Many methods are available for detecting specific alleles at human polymorphic loci. The preferred method for detecting a specific polymorphic allele will depend, in part, upon the molecular nature of the polymorphism. For example, the various allelic forms of the polymorphic locus may differ by a single base-pair of the DNA. Such single nucleotide polymorphisms (or SNPs) are major contributors to genetic variation, comprising some 80% of all known polymorphisms, and their density in the human genome is estimated to be on average 1 per 1,000 base pairs. SNPs are most frequently biallelic-occurring in only two different forms (although up to four different forms of an SNP, corresponding to the four different nucleotide bases occurring in DNA, are theoretically possible). Nevertheless, SNPs are mutationally more stable than other polymorphisms, making them suitable for association studies in which linkage disequilibrium between markers and an unknown variant is used to map disease-causing mutations. In addition, because SNPs typically have only two alleles, they can be genotyped by a simple plus/minus assay rather than a length measurement, making them more amenable to automation.

A variety of methods are available for detecting the presence of a particular single nucleotide polymorphic allele in an individual. Advancements in this field have provided accurate, easy, and inexpensive large-scale SNP genotyping. Most recently, for example, several new techniques have been described including dynamic allele-specific hybridization (DASH), microplate array diagonal gel electrophoresis (MADGE), pyrosequencing, oligonucleotide-specific ligation, the TaqMan system as well as various DNA “chip” technologies such as the Affymetrix SNP chips. These methods require amplification of the target genetic region, typically by PCR. Still other newly developed methods, based on the generation of small signal molecules by invasive cleavage followed by mass spectrometry or immobilized padlock probes and rolling-circle amplification, might eventually eliminate the need for PCR. Several of the methods known in the art for detecting specific single nucleotide polymorphisms are summarized below. The method of the present invention is understood to include all available methods.

Several methods have been developed to facilitate analysis of single nucleotide polymorphisms. In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA® is described by Goelet, P. et al. (PCT Appln. No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. is preferably a heterogeneous phase assay, in which the primer or the target molecule is immobilized to a solid phase.

Recently, several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A.-C., et al., Genomics 8:684-692 (1990); Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem. 208:171-175 (1993)). These methods differ from GBA® in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C., et al., Amer. J. Hum. Genet. 52:46-59 (1993)).

For mutations that produce premature termination of protein translation, the protein truncation test (PTT) offers an efficient diagnostic approach (Roest, et. al., (1993) Hum. Mol. Genet. 2:1719-21; van der Luijt, et. al., (1994) Genomics 20:1-4). For PTT, RNA is initially isolated from available tissue and reverse-transcribed, and the segment of interest is amplified by PCR. The products of reverse transcription PCR are then used as a template for nested PCR amplification with a primer that contains an RNA polymerase promoter and a sequence for initiating eukaryotic translation. After amplification of the region of interest, the unique motifs incorporated into the primer permit sequential in vitro transcription and translation of the PCR products. Upon sodium dodecyl sulfate-polyacrylamide gel electrophoresis of translation products, the appearance of truncated polypeptides signals the presence of a mutation that causes premature termination of translation. In a variation of this technique, DNA (as opposed to RNA) is used as a PCR template when the target region of interest is derived from a single exon.

Any cell type or tissue may be utilized to obtain nucleic acid samples for use in the diagnostics described herein. In a preferred embodiment, the DNA sample is obtained from a bodily fluid, e.g, blood, obtained by known techniques (e.g venipuncture) or saliva. Alternatively, nucleic acid tests can be performed on dry samples (e.g. hair or skin). When using RNA or protein, the cells or tissues that may be utilized must express an IL-1 gene.

Diagnostic procedures may also be performed in situ directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, NY).

In addition to methods that focus primarily on the detection of one nucleic acid sequence, profiles may also be assessed in such detection schemes. Fingerprint profiles may be generated, for example, by utilizing a differential display procedure, Northern analysis and/or RT-PCR.

A preferred detection method is allele specific hybridization using probes overlapping a region of at least one allele of an IL-1 proinflammatory haplotype and having about 5, 10, 20, 25, or 30 nucleotides around the mutation or polymorphic region. In a preferred embodiment of the invention, several probes capable of hybridizing specifically to other allelic variants involved in aging-related dermatologic disorders are attached to a solid phase support, e.g, a “chip” (which can hold up to about 250,000 oligonucleotides). Oligonucleotides can be bound to a solid support by a variety of processes, including lithography. Mutation detection analysis using these chips comprising oligonucleotides, also termed “DNA probe arrays” is described e.g., in Cronin et al. (1996) Human Mutation 7:244. In one embodiment, a chip comprises all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment.

These techniques may also comprise the step of amplifying the nucleic acid before analysis. Amplification techniques are known to those of skill in the art and include, but are not limited to cloning, polymerase chain reaction (PCR), polymerase chain reaction of specific alleles (ASA), ligase chain reaction (LCR), nested polymerase chain reaction, self sustained sequence replication (Guatelli, J. C. et al., 1990, Proc. Natl. Acad. Sci. USA 87:1874-1878), transcriptional amplification system (Kwoh, D. Y. et al., 1989, Proc. Natl. Acad. Sci. USA 86:1173-1177), and Q-B Replicase (Lizardi, P. M. et al., 1988, Bio/Technology 6:1197).

Amplification products may be assayed in a variety of ways, including size analysis, restriction digestion followed by size analysis, detecting specific tagged oligonucleotide primers in the reaction products, allele-specific oligonucleotide (ASO) hybridization, allele specific 5′ exonuclease detection, sequencing, hybridization, and the like.

PCR based detection means can include multiplex amplification of a plurality of markers simultaneously. For example, it is well known in the art to select PCR primers to generate PCR products that do not overlap in size and can be analyzed simultaneously. Alternatively, it is possible to amplify different markers with primers that are differentially labeled and thus can each be differentially detected. Of course, hybridization based detection means allow the differential detection of multiple PCR products in a sample. Other techniques are known in the art to allow multiplex analyses of a plurality of markers.

In a merely illustrative embodiment, the method includes the steps of (i) collecting a sample of cells from a patient, (ii) isolating nucleic acid (e.g., genomic, mRNA or both) from the cells of the sample, (iii) contacting the nucleic acid sample with one or more primers which specifically hybridize 5′ and 3′ to at least one allele of an IL-1 proinflammatory haplotype under conditions such that hybridization and amplification of the allele occurs, and (iv) detecting the amplification product. These detection schemes are especially useful for the detection of nucleic acid molecules if such molecules are present in very low numbers.

In a preferred embodiment of the subject assay, the allele of an IL-1 proinflammatory haplotype is identified by alterations in restriction enzyme cleavage patterns. For example, sample and control DNA is isolated, amplified (optionally), digested with one or more restriction endonucleases, and fragment length sizes are determined by gel electrophoresis.

In yet another embodiment, any of a variety of sequencing reactions known in the art can be used to directly sequence the allele. Exemplary sequencing reactions include those based on techniques developed by Maxim and Gilbert ((1977) Proc. Natl. Acad Sci USA 74:560) or Sanger (Sanger et al (1977) Proc. Nat. Acad. Sci. USA 74:5463). It is also contemplated that any of a variety of automated sequencing procedures may be utilized when performing the subject assays (see, for example Biotechniques (1995) 19:448), including sequencing by mass spectrometry (see, for example PCT publication WO 94/16101; Cohen et al. (1996) Adv Chromatogr 36:127-162; and Griffin et al. (1993) Appl Biochem Biotechnol 38:147-159). It will be evident to one of skill in the art that, for certain embodiments, the occurrence of only one, two or three of the nucleic acid bases need be determined in the sequencing reaction. For instance, A-track or the like, e.g., where only one nucleic acid is detected, can be carried out.

In a further embodiment, protection from cleavage agents (such as a nuclease, hydroxylamine or osmium tetroxide and with piperidine) can be used to detect mismatched bases in RNA/RNA or RNA/DNA or DNA/DNA heteroduplexes (Myers, et al. (1985) Science 230:1242). In general, the art technique of “mismatch cleavage” starts by providing heteroduplexes formed by hybridizing (labeled) RNA or DNA containing the wild-type allele with the sample. The double-stranded duplexes are treated with an agent which cleaves single-stranded regions of the duplex such as which will exist due to base pair mismatches between the control and sample strands. For instance, RNA/DNA duplexes can be treated with RNase and DNA/DNA hybrids treated with S1 nuclease to enzymatically digest the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, for example, Cotton et al (1988) Proc. Natl. Acad Sci USA 85:4397; and Saleeba et al (1992) Methods Enzymol. 217:286-295. In a preferred embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes). For example, the mutY enzyme of E. coli cleaves A at G/A mismatches and the thymidine DNA glycosylase from HeLa cells cleaves T at G/T mismatches (Hsu et al. (1994) Carcinogenesis 15:1657-1662). According to an exemplary embodiment, a probe based on an allele of an IL-1 locus haplotype is hybridized to a cDNA or other DNA product from a test cell(s). The duplex is treated with a DNA mismatch repair enzyme, and the cleavage products, if any, can be detected from electrophoresis protocols or the like. See, for example, U.S. Pat. No. 5,459,039.

In other embodiments, alterations in electrophoretic mobility will be used to identify an IL-1 locus allele. For example, single strand conformation polymorphism (SSCP) may be used to detect differences in electrophoretic mobility between mutant and wild type nucleic acids (Orita et al. (1989) Proc Natl. Acad. Sci. USA 86:2766, see also Cotton (1993) Mutat Res 285:125-144; and Hayashi (1992) Genet Anal Tech Appl 9:73-79). Single-stranded DNA fragments of sample and control IL-1 locus alleles are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In a preferred embodiment, the subject method utilizes heteroduplex analysis to separate double stranded heteroduplex molecules on the basis of changes in electrophoretic mobility (Keen et al. (1991) Trends Genet. 7:5).

In yet another embodiment, the movement of alleles in polyacrylamide gels containing a gradient of denaturant is assayed using denaturing gradient gel electrophoresis (DGGE) (Myers et al. (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:12753).

Examples of other techniques for detecting alleles include, but are not limited to, selective oligonucleotide hybridization, selective amplification, or selective primer extension. For example, oligonucleotide primers may be prepared in which the known mutation or nucleotide difference (e.g., in allelic variants) is placed centrally and then hybridized to target DNA under conditions which permit hybridization only if a perfect match is found (Saiki et al. (1986) Nature 324:163); Saiki et al (1989) Proc. Natl. Acad. Sci USA 86:6230). Such allele specific oligonucleotide hybridization techniques may be used to test one mutation or polymorphic region per reaction when oligonucleotides are hybridized to PCR amplified target DNA or a number of different mutations or polymorphic regions when the oligonucleotides are attached to the hybridizing membrane and hybridized with labelled target DNA.

Alternatively, allele specific amplification technology which depends on selective PCR amplification may be used in conjunction with the instant invention. Oligonucleotides used as primers for specific amplification may carry the mutation or polymorphic region of interest in the center of the molecule (so that amplification depends on differential hybridization) (Gibbs et al (1989) Nucleic Acids Res. 17:2437-2448) or at the extreme 3′ end of one primer where, under appropriate conditions, mismatch can prevent, or reduce polymerase extension (Prossner (1993) Tibtech 11:238. In addition it may be desirable to introduce a novel restriction site in the region of the mutation to create cleavage-based detection (Gasparini et al (1992) Mol. Cell. Probes 6:1). It is anticipated that in certain embodiments amplification may also be performed using Taq ligase for amplification (Barany (1991) Proc. Natl. Acad. Sci. USA 88:189). In such cases, ligation will occur only if there is a perfect match at the 3′ end of the 5′ sequence making it possible to detect the presence of a known mutation at a specific site by looking for the presence or absence of amplification.

In another embodiment, identification of the allelic variant is carried out using an oligonucleotide ligation assay (OLA), as described, e.g., in U.S. Pat. No. 4,998,617 and in Landegren, U. et al. ((1988) Science 241:1077-1080). The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al. (1990) Proc. Natl. Acad. Sci. USA 87:8923-27). In this method, PCR is used to achieve the exponential amplification of target DNA, which is then detected using OLA.

Several techniques based on this OLA method have been developed and can be used to detect alleles of an IL-1 locus haplotype. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. ((1996) Nucleic Acids Res 24: 3728), OLA combined with PCR permits typing of two alleles in a single microtiter well. By marking each of the allele-specific primers with a unique hapten, i.e. digoxigenin and fluorescein, each OLA reaction can be detected by using hapten specific antibodies that are labeled with different enzyme reporters, alkaline phosphatase or horseradish peroxidase. This system permits the detection of the two alleles using a high throughput format that leads to the production of two different colors.

Another embodiment of the invention is directed to kits for detecting a predisposition for developing a dermatologic disorder. This kit may contain one or more oligonucleotides, including 5′ and 3′ oligonucleotides that hybridize 5′ and 3′ to at least one allele of an IL-1 locus haplotype. PCR amplification oligonucleotides should hybridize between 25 and 2500 base pairs apart, preferably between about 100 and about 500 bases apart, in order to produce a PCR product of convenient size for subsequent analysis.

Particularly preferred primer pairs for use in the diagnostic method of the invention include the following: 5

(SEQ ID No. 5)

5′- ATG GTT TTA GAA ATC ATC AAG CCT AGG GCA- 3′
and

(SEQ ID No. 6)

5′- AAT GAA AGG AGG GGA GGA TGA CAG AAA TGT -3′;

(SEQ ID No. 7)

5′- TGG CAT TGA TCT GGT TCA TC-3′
and

(SEQ ID No. 8)

5′- GTT TAG GAA TCT TCC CAC TT-3′;

(SEQ ID No. 9)

5′- CTC AGG TGT CCT CGA AGA AAT CAA A -3′
and

(SEQ ID No. 10)

5′- GCT TTT TTG CTG TGA GTC CCG -3′;

(SEQ ID No. 11)

5′-CTC AGC AAC ACT CCT AT-3′
and

(SEQ ID No. 12)

5′-TCC TGG TCT GCA GCT AA-3′;

(SEQ ID No. 13)

5′-CTA TCT GAG GAA CAA ACT AGT AGC-3′
and

(SEQ ID No. 14)

5′-TAG GAC ATT GCA CCT AGG GTT TGT-3′;

(SEQ ID No. 15)

5′- ATT TTT TTA TAA ATC ATC AAG CCT AGG GCA -3′
and

(SEQ. ID No. 16)

5′- AAT TAA AGG AGG GAA GAA TGA CAG AAA TGT -3′;

(SEQ ID No. 17)

5′-AAG CTT GTT CTA CCA CCT GAA CTA GGC-3′
and

(SEQ ID No. 18)

5′-TTA CAT ATG AGC CTT CCA TG-3′.

The design of additional oligonucleotides for use in the amplification and detection of IL-1 polymorphic alleles by the method of the invention is facilitated by the availability of both updated sequence information from human chromosome 2q13—which contains the human IL-1 locus, and updated human polymorphism information available for this locus. For example, the DNA sequence for the IL-1A, IL-1B and IL-1RN is shown in FIGS. 1 (GenBank Accession No. X03833), 2 (GenBank Accession No. X04500) and 3 (GenBank Accession No. X64532) respectively. Suitable primers for the detection of a human polymorphism in these genes can be readily designed using this sequence information and standard techniques known in the art for the design and optimization of primers sequences. Optimal design of such primer sequences can be achieved, for example, by the use of commercially available primer selection programs such as Primer 2.1, Primer 3 or GeneFisher (See also, Nicklin M. H. J., Weith A. Duff G. W., “A Physical Map of the Region Encompassing the Human Interleukin-1, interleukin-1, and Interleukin-1 Receptor Antagonist Genes” Genomics 19: 382 (1995); Nothwang H. G., et al. “Molecular Cloning of the Interleukin-1 gene Cluster: Construction of an Integrated YAC/PAC Contig and a partial transcriptional Map in the Region of Chromosome 2q13” Genomics 41: 370 (1997); Clark, et al. (1986) Nucl. Acids. Res., 14:7897-7914 [published erratum appears in Nucleic Acids Res., 15:868 (1987) and the Genome Database (GDB) project at the URL http://www.gdb.org].

For use in a kit, oligonucleotides may be any of a variety of natural and/or synthetic compositions such as synthetic oligonucleotides, restriction fragments, cDNAs, synthetic peptide nucleic acids (PNAs), and the like. The assay kit and method may also employ labeled oligonucleotides to allow ease of identification in the assays. Examples of labels which may be employed include radio-labels, enzymes, fluorescent compounds, streptavidin, avidin, biotin, magnetic moieties, metal binding moieties, antigen or antibody moieties, and the like.

The kit may, optionally, also include DNA sampling means. DNA sampling means are well known to one of skill in the art and can include, but not be limited to substrates, such as filter papers, the AmpliCard® (University of Sheffield, Sheffield, England S10 2JF; Tarlow, J W, et al., J. of Invest. Dermatol. 103:387-389 (1994)) and the like; DNA purification reagents such as Nucleon® kits, lysis buffers, proteinase solutions and the like; PCR reagents, such as 10× reaction buffers, thermostable polymerase, dNTPs, and the like; and allele detection means such as the HinfI restriction enzyme, allele specific oligonucleotides, degenerate oligonucleotide primers for nested PCR from dried blood.

Resolving an individual's haplotype involves determining or inferring whether an allele is present on the maternal chromosome, paternal chromosome, both chromosomes, or neither. Haplotypic information includes the results of such a determination for multiple linked alleles. Methods for obtaining and using haplotype data have been previously disclosed in U.S. Pat. No. 6,931,326, U.S. Pat. No. 6,920,398, U.S. Pat. No. 7,141,373, and U.S. Pat. No. 6,951,721, the disclosures of which are incorporated herein by reference.

Age-Related Therapeutics and Pharmacogenomics

The ability to rapidly genotype patients promises to fundamentally change the testing and development of therapeutic or disease-preventative substances. Currently, the effectiveness of a substance for treating or preventing a disease is assessed by testing it on a pool of patients. While many variables in the patient pool are controlled for, the effects of genetic variability are not typically tested. Consequently, a drug may be found to be statistically ineffective when examined in a genetically diverse pool of patients and yet be highly effective for a select group of patients with particular genetic characteristics. Unless patients are separated by genotype, many drugs with great promise for selected populations are likely to be rejected as useless for the population as a whole.

Knowledge of particular alleles associated with aging-related dermatologic disorders, alone or in conjunction with information on other genetic defects contributing to aging-related dermatologic disorders (the genetic profile of aging-related dermatologic disorders) allows a customization of the therapy to the individual's genetic profile, the goal of “pharmacogenomics”. For example, as shown herein, subjects having an allele associated with aging-related dermatologic disorders, such as IL-1RN (+2018) allele 2 are predisposed to aging-related dermatologic disorders. Thus, comparison of a subject's IL-1 profile to the population profile for the disease, permits the selection or design of drugs that are expected to be safe and efficacious for a particular patient or patient population (i.e., a group of patients having the same genetic alteration).

The ability to target populations expected to show the highest clinical benefit, based on the IL-1 gene profile or the genetic profile of aging-related dermatologic disorders, can enable: 1) the repositioning of marketed drugs with disappointing market results; 2) the rescue of drug candidates whose clinical development has been discontinued as a result of safety or efficacy limitations, which are patient subgroup-specific; and 3) an accelerated and less costly development for drug candidates and more optimal drug labeling (e.g. since measuring the effect of various doses of an agent on an aging-related dermatologic disorder causative mutation is useful for optimizing effective dose).

In one embodiment, a subject's IL-1 genotype and aging-related dermatologic disorder predisposition may be used to tailor a recommended lifestyle, including, for example, changes in exercise and diet. The IL-1 genotype may also be used to recommend nutraceuticals that are predicted to benefit a subject having a particular IL-1 genotype and aging-related dermatologic disorder predisposition.

In another embodiment, subject genotypes and aging-related dermatologic disorder predispositions may be used to manage costs of therapy, by separating patients into groups that are likely or unlikely to benefit from one or more therapeutic regimen. Decisions about the appropriate therapeutic regimen for a subject may be made in view of that subject's grouping, and such procedures may decrease the numbers of patients receiving an unnecessary, ineffective or inappropriate therapeutic regimen. Patients may be separated solely on the basis of genotype or on the basis of genotype in combination with other forms of information, such as lifestyle, age, body-mass index, clinical history, other risk factors, etc. Patients may be sorted into more than one group.

IL-1 Production and Molecular Signaling Pathways

To better understand likely targets for therapeutic intervention and likely aging-related dermatologic disorder biomarkers, it is necessary to understand general mechanisms for IL-1 signaling and production. IL-1 is part of a complex web of inter- and intra-cellular signaling events. Many proteins are involved in the inflammatory response and also in immune responses more generally. A partial list includes the interleukins, TNF, NF-κB, the immunoglobulins, clotting factors, lipoxygenases, as well as attendant receptors, antagonists and processing enzymes for the above.

The IL-1 polypeptides, IL-1α and IL-1β, are abundantly produced by activated macrophages that have been stimulated with bacterial lipopolysaccharide (LPS), TNF, IL-1 itself, other macrophage-derived cytokines, or contact with CD4⁺ T cells. The IL-1 promoter contains several regulatory elements including a cAMP responsive element, an AP-1 binding site and an NF-κB binding site. Both and AP-1 (Jun and Fos) must be activated and translocated to the nucleus in order to regulate transcription. NF-κB is normally retained in the cytoplasm through binding with IκB. The NF-κB-IκB complex is disrupted by phosphorylation of IkB. IkB phosphorylation can be regulated by signaling from cell-surface receptors via activation of mitogen-activated protein kinase (MAP kinase) pathways and other kinase pathways. Jun and Fos are also substrates for regulatory kinases, such as JNK, in the case of Jun.

The IL-1A and B transcripts are translated into pro-proteins by a process that may also be regulated by MAP kinase pathways. Inhibitors of MAP kinase phosphorylation such as trebufelone decrease translation of IL-1 transcripts. The IL-1α and β precursor proteins require myristoylation for localization to the membrane and conversion to mature IL-1 by the Interleukin Converting Enzyme (ICE), or Caspose I. Other extracellular proteases may also play a minor role in IL-1 maturation, including trypsin, elastase, chymotrypsin and mast cell chymase. ICE can be inhibited by several agents including the eICE isoform, antibodies to the ICE α, β and gamma. isoforms, the cow pox-produced Crm-A protein and an endogenous tetrapeptide competitive inhibitor.

Mature IL-1α and IL-1β have similar activities and interact with the same receptors. The primary receptor for these factors is the type I IL-1 receptor. The active signaling complex consists of the IL-1 ligand, the type I receptor and the IL-1 receptor accessory protein. A type II receptor, as well as soluble forms of the type I and type II receptors appear to act as decoy receptors to compete for bioavailable IL-1. In addition, a natural inhibitor of IL-1 signaling, IL-1 receptor antagonist, is produced by monocytes. IL-1ra is also produced by hepatocytes and is a major component of the acute phase proteins produced in the liver and secreted into the circulation to regulate immune and inflammatory responses.

The IL-1 signaling complex activates several intracellular signal transduction pathways, including the activities of NF-κB and AP-1 described above. In signaling, IL-1 influences the activity of a host of factors including: PI-3 kinase, phospholipase A2, protein kinase C, the JNK pathway, 5-lipoxygenase, cyclooxygenase 2, p38 MAP kinase, p42/44 MAP kinase, p54 MAP kinase, Rac, Ras, TRAF-6, TRAF-2 and many others. IL-1 also affects expression of a large number of genes including: members of the IL-1 gene cluster, TNF, other interleukin genes (2, 3, 6, 8, 12, 2R, 3R and SR), TGF-β, fibrinogen, matrix metalloprotease 1, collagen, elastase, leukemia inhibiting factor, IFN α, β, gamma., COX-2, inducible nitric oxide synthase, metallothioneins, and many more.

Aging-Related Dermatologic Disorders Associated Biomarkers

In addition to having genetic tests for aging-related dermatologic disorders, it would be desirable to have tests for monitoring a subject's progression towards or during aging-related dermatologic disorders. In other words, certain biomarkers may be indicative of the timing and/or progression of early onset of aging-related conditions. It would be desirable to be able to identify these biomarkers and monitor them to provide information about the onset and progression of aging-related conditions. It is particularly desirable to find biomarkers that are tailored to the subject's genotype.

In a preferred embodiment, biomarkers likely to be associated with aging-related dermatologic disorders may be identified by using subjects or cells comprising differing IL-1 genotypes. A set of biomarkers may be examined in a subject or cell having an aging-related dermatologic disorder-associated allele, such as IL-1RN (+2018) allele 2, IL-1B (−511) allele 2. The same set of biomarkers can be examined in another subject or cell not having an aging-related dermatologic disorders -associated allele. Biomarkers that show a difference dependent upon the IL-1 genotype are likely to be useful for predicting aging-related dermatologic disorders. These differences constitute ARDD-associated phenotypes.

The association between certain biomarkers and aging-related dermatologic disorders may be further established by performing trials wherein certain biomarkers are measured in a population of subjects of various ages, some of which may have already begun to evince aging-related conditions. Optionally, multiple measurements may be done over time as subjects age. Preferably, the presence or absence of ARDD-associated alleles is determined in the subjects. Standard statistical methods may be used to determine the correlation between certain biomarkers and the early onset of aging-related conditions.

Measurements of ARDD-associated biomarkers may be used as an indicator of a subject's current risk of developing ARDD or as an indicator of progression towards or through the aging process.

With respect to cells, biomarkers may be essentially any aspect of cell function, for example: levels or rate of production of signaling molecules, transcription factors, intermediate metabolites, cytokines, prostanoids, steroid hormones (eg. estrogen, progesterone, androstenedione or testosterone), gonadotropins (eg. LH and FSH), gene transcripts, post-translational modifications of proteins, gonadotropin releasing hormone (GnRH), catecholamines (eg. dopamine or norepinephrine), opioids, activin, inhibin, as well as IL-1 bioactivities. Biomarkers may include whole genome analysis of transcript levels or whole proteome analysis of protein levels and/or modifications. Additionally, biomarkers may be reporter genes. For example, an IL-1 promoter or an IL-1 promoter comprising an ARDD-associated allele can be operationally linked to a reporter gene. In an alternative method, the promoter can be an IL-1-regulated promoter, such as IL-8. In this manner, the activity of the reporter gene is reflective of the activity of the promoter. Suitable reporter genes include luciferase (luc), GUS, LacZ, green fluorescent protein (GFP) (and variants thereof, such as RFP, CFP, YFP and BFP), or essentially any other gene that is easily detected. In subjects, biomarkers can be, for example, any of the above as well as electrocardiogram parameters, pulmonary function, IL-6 activities, urine parameters or tissue parameters. Other preferred biomarkers include factors involved in immune and inflammatory responses, as well as factors involved in IL-1 production and signaling, as described above.

Aging-Related Dermatologic Disorder Therapeutics

An aging-related dermatologic disorder therapeutic or ARDD therapeutic may comprise any type of compound, including a protein, peptide, peptidomimetic, small molecule, nucleic acid, or nutraceutical. In preferred embodiments, an ARDD therapeutic is a modulator of a factor involved in IL-1 production or signaling. In a particularly preferred embodiment, an ARDD therapeutic is a modulator of IL-1 bioactivity (e.g. IL-1, IL-1β or an IL-1 receptor agonist or antagonist). Preferred agonists include nucleic acids (e.g. encoding an IL-1 protein or a gene that is up- or down-regulated by an IL-1 protein), protein (e.g. IL-1 proteins or a protein that is up- or down-regulated by an IL-1 protein) or a small molecule (e.g. that regulates expression of an IL-1 protein). Preferred antagonists, which can be identified, for example, using the assays described herein, include nucleic acids (e.g. single (antisense) or double stranded (triplex) DNA or PNA and ribozymes), protein (e.g. antibodies) and small molecules or nutraceuticals that act to suppress or inhibit IL-1 transcription and/or IL-1 activity.

An ARDD therapeutic may also be any cosmetic or pharmaceutical agents useful for the treatment of aging-related dermatologic disorders. These agents may include: agents that improve or eradicate age spots, keratoses and wrinkles; local analgesics and anesthetics; anti-acne agents; anti-bacterials; anti-yeast agents; anti-fungal agents; anti-viral agents; anti-dandruff agents; anti-dermatitis agents; anti-histamine agents; anti-pruritic agents; anti-emetics; anti-motion sickness agents; anti-inflammatory agents; anti-hyperkeratolytic agents; antiperspirants; anti-psoriatic agents; anti-seborrheic agents; hair conditioners and hair treatment agents; anti-aging and anti-wrinkle agents; sunblock and sunscreen agents; skin lightening agents; depigmenting agents; vitamins; corticosteroids; tanning agents; hormones; retinoids; topical cardiovascular agents; hydroxyacids, ketoacids and related compounds; phenyl α acyloxyalkanoic acids and derivatives thereof; and N-acetyl-aldosamines, N-acetylamino acids and related N-acetyl compounds. Additional cosmetic or pharmaceutical agents useful of the treatment of aging-related dermatologic disorders are disclosed in U.S. Patent Publication No. 2002/0028227, which is incorporated herein by reference in its entirety.

More particularly, the ARDD therapeutic may be any cosmetic or pharmaceutical including the one or more of the following: aclovate, acyclovir, acetylsalicylic acid, adapalene, albuterol, aluminum acetate, aluminum chloride, aluminum hydroxide, aluminum chlorohydroxide, amantadine, aminacrine, aminobenzoic acid (PABA), aminocaproic acid, aminosalicylic acid, amitriptyline, anthralin, ascorbic acid, ascoryl palimate, atropine, azelaic acid, bacitracin, bemegride, beclomethasone dipropionate, benzophenone, benzoyl peroxide, βmethasone dipropionate, βmethasone valerate, brompheniramine, bupivacaine, butoconazole, calcipotriene, camphor, capsaicin, carbamide peroxide, chitosan, chlorhexidine, chloroxylenol, chlorpheniramine, ciclopirox, clemastine, clindamycin, clioquinol, cloβsol propionate, clotrimazole, coal tar, cromolyn, crotamiton, cycloserine, dehydroepiandrosterone, desoximetasone, dexamethasone, diphenhydramine, doxypin, doxylamine, dyclonine, econazole, erythromycin, estradiol, ethinyl estradiol, fluocinonide, fluocinolone acetonide, 5-fluorouracil, griseofulvin, guaifenesin, haloprogin, hexylresorcinol, homosalate, hydrocortisone, hydrocortisone 21-acetate, hydrocortisone 17-valerate, hydrocortisone 17-butyrate, hydrogen peroxide, hydroquinone, hydroquinone monoether, hydroxyzine, ibuprofen, ichthammol, imiquimod, indomethacin, ketoconazole, ketoprofen, kojic acid, lidocaine, meclizine, meclocycline, menthol, mepivacaine, methyl nicotinate, methyl salicylate, metronidazole, miconazole, minocycline, minoxidil, monobenzone, mupirocin, naftifine, naproxen, neomycin, nystatin, octyl methoxycinnamate, octyl salicylate, oxybenzone, oxiconazole, oxymetazoline, padimate O, permethrin, pheniramine, phenol, phenylephrine, phenylpropanolamine, piperonyl butoxide, podophyllin, podofilox, povidone iodine, pramoxine, prilocalne, procaine, promethazine propionate, propranolol, pseudoephedrine, pyrethrin, pyrilamine, resorcinol, retinal, 13-cis retinoic acid, retinoic acid, retinol, retinyl acetate, retinyl palmitate, salicylamide, salicylic acid, selenium sulfide, shale tar, sulconazole, sulfur, sulfadiazine, tazarotene, terbinafine, terconazole, tetracaine, tetracycline, tetrahydrozoline, thymol, tioconazole, tolnaftate, triamcinolone diacetate, triamcinolone acetonide, triamcinolone hexacetonide, triclosan, triprolidine, undecylenic acid, urea, vitamin E acetate, wood tar, zinc pyrithione, glycolic acid, lactic acid, methyllactic acid, 4-hydroxy-mandelic acid, mandelic acid, gluconolactone, N-acetyl-glucosamine, N-acetyl-proline, phenyl 2-acetoxyethanoic acid and diphenyl 2-acetoxyethanoic acid.

In Vivo and Cell-Based Screening Assays

Based on the identification of IL-1 mutations that cause or contribute to aging-related dermatologic disorders, the invention further features in vivo and cell-based assays, e.g., for identifying ARDD therapeutics. In one embodiment, a cell having an ARDD-associated allele is contacted with a test compound and at least one biomarker is measured. If at least one biomarker changes such that the phenotype of the cell now more closely resembles that of a cell that does not have an ARDD-associated allele, then the test substance is likely to be effective as an ARDD therapeutic.

As an illustrative example, suppose that an IL-1 allele associated with ARDD causes cells having that allele to overproduce an IL-1 polypeptide. Levels of the IL-1 polypeptide are used as a biomarker in this case. Treatment with a test substance causes the cells to produce the IL-1 polypeptide at a lower level, more closely resembling IL-1 polypeptide production in a cell that does not have an ARDD-associated allele. Accordingly, the test substance is likely to be effective as an ARDD therapeutic. In this manner, test substances with allele-specific effects may be identified. The specificity of the compound vis a vis the IL-1 signaling pathway can, if desired, be confirmed by various control analysis, e.g., measuring the expression of one or more control genes. In particular, this assay can be used to determine the efficacy of IL-1 antisense, ribozyme and triplex compounds.

In another variation a cell is contacted with a test compound and an IL-1 protein and the interaction between the test compound and the IL-1 receptor or between the IL-1 protein (preferably a tagged IL-1 protein) and the IL-1 receptor is detected, e.g., by using a microphysiometer (McConnell et al. (1992) Science 257:1906). An interaction between the IL-1 receptor and either the test compound or the IL-1 protein is detected by the microphysiometer as a change in the acidification of the medium. This assay system thus provides a means of identifying molecular antagonists which, for example, function by interfering with IL-1 protein-IL-1 receptor interactions, as well as molecular agonist which, for example, function by activating an IL-1 receptor.

Essentially any culturable cell type can be used for the cell-based assays. In particular, cells may be immune cells such as monocytes, macrophages or thymocytes, or other cell types such as fibroblasts, keratinocytes, melanocytes, or cells derived from female reproductive organs. Preferrably cells will express an IL-1 receptor.

In another variation, a subject having an ARDD-associated allele is contacted with a test compound and at least one biomarker is measured. If at least one biomarker changes such that the phenotype of the cell now more closely resembles that of a cell that does not have an ARDD-associated allele, then the test substance is likely to be effective as an ARDD therapeutic. The subject may be a human or a transgenic non-human animal.

In preferred embodiments, cellular or in vivo assays are used to identify compounds which modulate expression of an IL-1 gene, modulate translation of an IL-1 mRNA, or which modulate the stability or activity of an IL-1 mRNA or protein. Accordingly, in one embodiment, a cell which is capable of producing IL-1 protein is incubated with a test compound and the amount of IL-1 protein produced in the cell medium is measured and compared to that produced from a cell which has not been contacted with the test compound. In another variation, an IL-1 bioactivity is measured and compared to the bioactivity measured in a cell which has not been contacted with a test compound. Additionally, the effects of test substances on different cells containing various IL-1 alleles may be compared.

Cell-Free Assays

Cell-free assays can also be used to identify compounds which are capable of interacting with an IL-1 protein, to thereby modify the activity of the L-1 protein. Such a compound can, e.g., modify the structure of an IL-1 protein thereby affecting its ability to bind to an IL-1 receptor. In a preferred embodiment, cell-free assays for identifying such compounds consist essentially in a reaction mixture containing an IL-1 protein and a test compound or a library of test compounds in the presence or absence of a binding partner. A test compound can be, e.g., a derivative of an IL-1 binding partner, e.g., a biologically inactive target peptide, or a small molecule.

Accordingly, one exemplary screening assay of the present invention includes the steps of contacting an IL-1 protein or functional fragment thereof with a test compound or library of test compounds and detecting the formation of complexes. For detection purposes, the molecule can be labeled with a specific marker and the test compound or library of test compounds labeled with a different marker. Interaction of a test compound with an IL-1 protein or fragment thereof can then be detected by determining the level of the two labels after an incubation step and a washing step. The presence of two labels after the washing step is indicative of an interaction.

An interaction between molecules can also be identified by using real-time BIA (Biomolecular Interaction Analysis, Pharmacia Biosensor AB) which detects surface plasmon resonance (SPR), an optical phenomenon. Detection depends on changes in the mass concentration of macromolecules at the biospecific interface, and does not require any labeling of interactants. In one embodiment, a library of test compounds can be immobilized on a sensor surface, e.g., which forms one wall of a micro-flow cell. A solution containing the IL-1β protein or functional fragment thereof is then flown continuously over the sensor surface. A change in the resonance angle as shown on a signal recording, indicates that an interaction has occurred. This technique is further described, e.g., in BIAtechnology Handbook by Pharmacia.

Another exemplary screening assay of the present invention includes the steps of (a) forming a reaction mixture including: (i) an IL-1 protein, (ii) an IL-1 receptor, and (iii) a test compound; and (b) detecting interaction of the IL-1 protein and IL-1 receptor. A statistically significant change (potentiation or inhibition) in the interaction of the IL-1 protein and IL-1 receptor in the presence of the test compound, relative to the interaction in the absence of the test compound, indicates a potential antagonist (inhibitor) of IL-1 bioactivity for the test compound. The compounds of this assay can be contacted simultaneously. Alternatively, an IL-1 protein can first be contacted with a test compound for an appropriate amount of time, following which the IL-1β receptor is added to the reaction mixture. The efficacy of the compound can be assessed by generating dose response curves from data obtained using various concentrations of the test compound. Moreover, a control assay can also be performed to provide a baseline for comparison.

Complex formation between an IL-1 protein and IL-1 receptor may be detected by a variety of techniques. Modulation of the formation of complexes can be quantitated using, for example, detectably labeled proteins such as radiolabeled, fluorescently labeled, or enzymatically labeled IL-1 protein or IL-1 receptors, by immunoassay, or by chromatographic detection.

Typically, it will be desirable to immobilize either IL-1 protein or the IL-1 receptor to facilitate separation of complexes from uncomplexed forms of one or both of the proteins, as well as to accommodate automation of the assay. Binding of IL-1 protein and IL-1 receptor can be accomplished in any vessel suitable for containing the reactants. Examples include microtitre plates, test tubes, and micro-centrifuge tubes. In one embodiment, a fusion protein can be provided which adds a domain that allows the protein to be bound to a matrix. For example, glutathione-S-transferase/IL-1 (GST/IL-1β) fusion proteins can be adsorbed onto glutathione sepharose beads (Sigma Chemical, St. Louis, Mo.) or glutathione derivatized microtitre plates, which are then combined with the IL-1 receptor, e.g. an 35S-labeled IL-1 receptor, and the test compound, and the mixture incubated under conditions conducive to complex formation, e.g. at physiological conditions for salt and pH, though slightly more stringent conditions may be desired. Following incubation, the beads are washed to remove any unbound label, and the matrix immobilized and radiolabel determined directly (e.g. beads placed in scintilant), or in the supernatant after the complexes are subsequently dissociated. Alternatively, the complexes can be dissociated from the matrix, separated by SDS-PAGE, and the level of IL-1 protein or IL-1 receptor found in the bead fraction quantitated from the gel using standard electrophoretic techniques such as described in the appended examples. Other techniques for immobilizing proteins on matrices are also available for use in the subject assay. For instance, either IL-1 protein or IL-1 receptor can be immobilized utilizing conjugation of biotin and streptavidin.

Transgenic Animals

As described above, transgenic animals can be made for example, to assist in screening for ARDD therapeutics. Transgenic animals of the invention can include non-human animals containing an IL-1 mutation, which is causative of aging-related dermatologic disorders in humans, under the control of an appropriate IL-1 promoter or under the control of a heterologous promoter. Transgenic animals of the invention can also include an IL-1 gene expressed at such a level as to create an ARDD phenotype. To compare the effects of different IL-1 alleles, transgenic animals may be generated with a variety of IL-1 alleles and differences in ARDD phenotype can be identified. By testing different alleles and different expression levels, an animal with an ARDD phenotype optimal for testing candidate drugs can be generated and identified.

The transgenic animals can also be animals containing a transgene, such as reporter gene, under the control of an IL-1 promoter or fragment thereof. These animals are useful, e.g., for identifying drugs that modulate production of an IL-1, such as by modulating gene expression. In certain variations, the IL-1 allele may be a promoter mutation. In this case it is particularly desirable to operationally fuse the altered promoter to a suitable reporter gene.

Methods for obtaining transgenic non-human animals are well known in the art. In preferred embodiments, the expression of the ARDD causative mutation is restricted to specific subsets of cells, tissues or developmental stages utilizing, for example, cis-acting sequences that control expression in the desired pattern. In the present invention, such mosaic expression of an IL-1 protein can be essential for many forms of lineage analysis and can additionally provide a means to assess the effects of, for example, expression level which might grossly alter development in small patches of tissue within an otherwise normal embryo. Toward this end, tissue-specific regulatory sequences and conditional regulatory sequences can be used to control expression of the IL-1 mutation in certain spatial patterns. Moreover, temporal patterns of expression can be provided by, for example, conditional recombination systems or prokaryotic transcriptional regulatory sequences. Genetic techniques, which allow for the expression of IL-1 mutation can be regulated via site-specific genetic manipulation in vivo, are known to those skilled in the art.

The transgenic animals of the present invention all include within a plurality of their cells an ARDD causative mutation transgene of the present invention, which transgene alters the phenotype of the “host cell”. In an illustrative embodiment, either the cre/loxP recombinase system of bacteriophage P1 (Lakso et al. (1992) PNAS 89:6232-6236; Orban et al. (1992) PNAS 89:6861-6865) or the FLP recombinase system of Saccharomyces cerevisiae (O'Gorman et al. (1991) Science 251:1351-1355; PCT publication WO 92/15694) can be used to generate in vivo site-specific genetic recombination systems. Cre recombinase catalyzes the site-specific recombination of an intervening target sequence located between loxP sequences. loxP sequences are 34 base pair nucleotide repeat sequences to which the Cre recombinase binds and are required for Cre recombinase mediated genetic recombination. The orientation of loxP sequences determines whether the intervening target sequence is excised or inverted when Cre recombinase is present (Abremski et al. (1984) J. Biol. Chem. 259:1509-1514); catalyzing the excision of the target sequence when the loxP sequences are oriented as direct repeats and catalyzes inversion of the target sequence when loxP sequences are oriented as inverted repeats.

Accordingly, genetic recombination of the target sequence is dependent on expression of the Cre recombinase. Expression of the recombinase can be regulated by promoter elements which are subject to regulatory control, e.g., tissue-specific, developmental stage-specific, inducible or repressible by externally added agents. This regulated control will result in genetic recombination of the target sequence only in cells where recombinase expression is mediated by the promoter element. Thus, the activation of expression of the dermatologic disorder causative mutation transgene can be regulated via control of recombinase expression.

Use of the cre/loxP recombinase system to regulate expression of an ARDD causative mutation transgene requires the construction of a transgenic animal containing transgenes encoding both the Cre recombinase and the subject protein. Animals containing both the Cre recombinase and the ARDD causative mutation transgene may be provided through the construction of “double” transgenic animals. A convenient method for providing such animals is to mate two transgenic animals each containing a transgene.

Similar conditional transgenes can be provided using prokaryotic promoter sequences which require prokaryotic proteins to be simultaneous expressed in order to facilitate expression of the transgene. Exemplary promoters and the corresponding trans-activating prokaryotic proteins are given in U.S. Pat. No. 4,833,080.

Moreover, expression of the conditional transgenes can be induced by gene therapy-like methods wherein a gene encoding the transactivating protein, e.g. a recombinase or a prokaryotic protein, is delivered to the tissue and caused to be expressed, such as in a cell-type specific manner. By this method, the transgene could remain silent into adulthood until “turned on” by the introduction of the transactivator.

In an exemplary embodiment, the “transgenic non-human animals” of the invention are produced by introducing transgenes into the germline of the non-human animal. Embryonal target cells at various developmental stages can be used to introduce transgenes. Different methods are used depending on the stage of development of the embryonal target cell. The specific line(s) of any animal used to practice this invention are selected for general good health, good embryo yields, good pronuclear visibility in the embryo, and good reproductive fitness. In addition, the haplotype is a significant factor. For example, when transgenic mice are to be produced, strains such as C57BL/6 or FVB lines are often used (Jackson Laboratory, Bar Harbor, Me.). Preferred strains are those with H-2b, H-2d or H-2q haplotypes such as C57BL/6 or DBA/1. The line(s) used to practice this invention may themselves be transgenics, and/or may be knockouts (i.e., obtained from animals which have one or more genes partially or completely suppressed).

In one embodiment, the transgene construct is introduced into a single stage embryo. The zygote is the best target for microinjection. In the mouse, the male pronucleus reaches the size of approximately 20 micrometers in diameter which allows reproducible injection of 1-2 pl of DNA solution. The use of zygotes as a target for gene transfer has a major advantage in that in most cases the injected DNA will be incorporated into the host gene before the first cleavage (Brinster et al. (1985) PNAS 82:4438-4442). As a consequence, all cells of the transgenic animal will carry the incorporated transgene. This will in general also be reflected in the efficient transmission of the transgene to offspring of the founder since 50% of the germ cells will harbor the transgene.

Normally, fertilized embryos are incubated in suitable media until the pronuclei appear. At about this time, the nucleotide sequence comprising the transgene is introduced into the female or male pronucleus as described below. In some species such as mice, the male pronucleus is preferred. It is most preferred that the exogenous genetic material be added to the male DNA complement of the zygote prior to its being processed by the ovum nucleus or the zygote female pronucleus. It is thought that the ovum nucleus or female pronucleus release molecules which affect the male DNA complement, perhaps by replacing the protamines of the male DNA with histones, thereby facilitating the combination of the female and male DNA complements to form the diploid zygote.

Thus, it is preferred that the exogenous genetic material be added to the male complement of DNA or any other complement of DNA prior to its being affected by the female pronucleus. For example, the exogenous genetic material is added to the early male pronucleus, as soon as possible after the formation of the male pronucleus, which is when the male and female pronuclei are well separated and both are located close to the cell membrane. Alternatively, the exogenous genetic material could be added to the nucleus of the sperm after it has been induced to undergo decondensation. Sperm containing the exogenous genetic material can then be added to the ovum or the decondensed sperm could be added to the ovum with the transgene constructs being added as soon as possible thereafter.

Introduction of the transgene nucleotide sequence into the embryo may be accomplished by any means known in the art such as, for example, microinjection, electroporation, or lipofection. Following introduction of the transgene nucleotide sequence into the embryo, the embryo may be incubated in vitro for varying amounts of time, or reimplanted into the surrogate host, or both. in vitro incubation to maturity is within the scope of this invention. One common method in to incubate the embryos in vitro for about 1-7 days, depending on the species, and then reimplant them into the surrogate host.

For the purposes of this invention a zygote is essentially the formation of a diploid cell which is capable of developing into a complete organism. Generally, the zygote will be comprised of an egg containing a nucleus formed, either naturally or artificially, by the fusion of two haploid nuclei from a gamete or gametes. Thus, the gamete nuclei must be ones which are naturally compatible, i.e., ones which result in a viable zygote capable of undergoing differentiation and developing into a functioning organism. Generally, a euploid zygote is preferred. If an aneuploid zygote is obtained, then the number of chromosomes should not vary by more than one with respect to the euploid number of the organism from which either gamete originated.

In addition to similar biological considerations, physical ones also govern the amount (e.g., volume) of exogenous genetic material which can be added to the nucleus of the zygote or to the genetic material which forms a part of the zygote nucleus. If no genetic material is removed, then the amount of exogenous genetic material which can be added is limited by the amount which will be absorbed without being physically disruptive. Generally, the volume of exogenous genetic material inserted will not exceed about 10 picoliters. The physical effects of addition must not be so great as to physically destroy the viability of the zygote. The biological limit of the number and variety of DNA sequences will vary depending upon the particular zygote and functions of the exogenous genetic material and will be readily apparent to one skilled in the art, because the genetic material, including the exogenous genetic material, of the resulting zygote must be biologically capable of initiating and maintaining the differentiation and development of the zygote into a functional organism.

The number of copies of the transgene constructs which are added to the zygote is dependent upon the total amount of exogenous genetic material added and will be the amount which enables the genetic transformation to occur. Theoretically only one copy is required; however, generally, numerous copies are utilized, for example, 1,000-20,000 copies of the transgene construct, in order to insure that one copy is functional. As regards the present invention, there will often be an advantage to having more than one functioning copy of each of the inserted exogenous DNA sequences to enhance the phenotypic expression of the exogenous DNA sequences.

Any technique which allows for the addition of the exogenous genetic material into nucleic genetic material can be utilized so long as it is not destructive to the cell, nuclear membrane or other existing cellular or genetic structures. The exogenous genetic material is preferentially inserted into the nucleic genetic material by microinjection. Microinjection of cells and cellular structures is known and is used in the art.

Reimplantation is accomplished using standard methods. Usually, the surrogate host is anesthetized, and the embryos are inserted into the oviduct. The number of embryos implanted into a particular host will vary by species, but will usually be comparable to the number of off spring the species naturally produces.

Transgenic offspring of the surrogate host may be screened for the presence and/or expression of the transgene by any suitable method. Screening is often accomplished by Southern blot or Northern blot analysis, using a probe that is complementary to at least a portion of the transgene. Western blot analysis using an antibody against the protein encoded by the transgene may be employed as an alternative or additional method for screening for the presence of the transgene product. Typically, DNA is prepared from tail tissue and analyzed by Southern analysis or PCR for the transgene. Alternatively, the tissues or cells believed to express the transgene at the highest levels are tested for the presence and expression of the transgene using Southern analysis or PCR, although any tissues or cell types may be used for this analysis.

Alternative or additional methods for evaluating the presence of the transgene include, without limitation, suitable biochemical assays such as enzyme and/or immunological assays, histological stains for particular marker or enzyme activities, flow cytometric analysis, and the like. Analysis of the blood may also be useful to detect the presence of the transgene product in the blood, as well as to evaluate the effect of the transgene on the levels of various types of blood cells and other blood constituents.

Progeny of the transgenic animals may be obtained by mating the transgenic animal with a suitable partner, or by in vitro fertilization of eggs and/or sperm obtained from the transgenic animal. Where mating with a partner is to be performed, the partner may or may not be transgenic and/or a knockout; where it is transgenic, it may contain the same or a different transgene, or both. Alternatively, the partner may be a parental line. Where in vitro fertilization is used, the fertilized embryo may be implanted into a surrogate host or incubated in vitro, or both. Using either method, the progeny may be evaluated for the presence of the transgene using methods described above, or other appropriate methods.

The transgenic animals produced in accordance with the present invention will include exogenous genetic material. Further, in such embodiments the sequence will be attached to a transcriptional control element, e.g., a promoter, which preferably allows the expression of the transgene product in a specific type of cell.

Retroviral infection can also be used to introduce the transgene into a non-human animal. The developing non-human embryo can be cultured in vitro to the blastocyst stage. During this time, the blastomeres can be targets for retroviral infection (Jaenich, R. (1976) PNAS 73:1260-1264). Efficient infection of the blastomeres is obtained by enzymatic treatment to remove the zona pellucida (Manipulating the Mouse Embryo, Hogan eds. (Cold Spring Harbor Laboratory Press, Cold Spring Harbor, 1986). The viral vector system used to introduce the transgene is typically a replication-defective retrovirus carrying the transgene (Jahner et al. (1985) PNAS 82:6927-6931; Van der Putten et al. (1985) PNAS 82:6148-6152). Transfection is easily and efficiently obtained by culturing the blastomeres on a monolayer of virus-producing cells (Van der Putten, supra; Stewart et al. (1987) EMBO J. 6:383-388). Alternatively, infection can be performed at a later stage. Virus or virus-producing cells can be injected into the blastocoele (Jahner et al. (1982) Nature 298:623-628). Most of the founders will be mosaic for the transgene since in corporation occurs only in a subset of the cells which formed the transgenic non-human animal. Further, the founder may contain various retroviral insertions of the transgene at different positions in the genome which generally will segregate in the offspring. In addition, it is also possible to introduce transgenes into the germ line by intrauterine retroviral infection of the midgestation embryo (Jahner et al. (1982) supra).

A third type of target cell for transgene introduction is the embryonal stem cell (ES). ES cells are obtained from pre-implantation embryos cultured in vitro and fused with embryos (Evans et al. (1981) Nature 292:154-156; Bradley et al. (1984) Nature 309:255-258; Gossler et al. (1986) PNAS 83: 9065-9069; and Robertson et al. (1986) Nature 322:445-448). Transgenes can be efficiently introduced into the ES cells by DNA transfection or by retrovirus-mediated transduction. Such transformed ES cells can thereafter be combined with blastocysts from a non-human animal. The ES cells thereafter colonize the embryo and contribute to the germ line of the resulting chimeric animal. For review see Jaenisch, R. (1988) Science 240:1468-1474.

Effective Dose

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic induces are preferred. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Formulation and Use

Pharmaceutical compositions for use in accordance with the present invention may be formulated in conventional manner using one or more physiologically acceptable carriers or excipients. Thus, the compounds and their physiologically acceptable salts and solvates may be formulated for administration by, for example, injection, inhalation or insufflation (either through the mouth or the nose) or oral, buccal, parenteral or rectal administration.

For such therapy, the compounds of the invention can be formulated for a variety of loads of administration, including systemic and topical or localized administration. Techniques and formulations generally may be found in Remmington's Pharmaceutical Sciences, Meade Publishing Co., Easton, Pa. For systemic administration, injection is preferred, including intramuscular, intravenous, intraperitoneal, and subcutaneous. For injection, the compounds of the invention can be formulated in liquid solutions, preferably in physiologically compatible buffers such as Hank's solution or Ringer's solution. In addition, the compounds may be formulated in solid form and redissolved or suspended immediately prior to use. Lyophilized forms are also included.

For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., ationd oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

The compounds may also be formulated for topical administration. Compositions comprising an ARDD therapeutic may be formulated as solution, gel, lotion, cream, ointment, shampoo, spray, stick, powder, masque, mouth rinse or wash, vaginal gel or preparation, or other form acceptable for use on skin, nail, hair, oral mucosa, vaginal mucosa, mouth or gums.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration bile salts and fusidic acid derivatives. in addition, detergents may be used to facilitate permeation. Transmucosal administration may be through nasal sprays or using suppositories. For topical administration, the oligomers of the invention are formulated into ointments, salves, gels, or creams as generally known in the art. A wash solution can be used locally to treat an injury or inflammation to accelerate healing.

In clinical settings, a gene delivery system for the ARDD therapeutic gene can be introduced into a patient by any of a number of methods, each of which is familiar in the art. For instance, a pharmaceutical preparation of the gene delivery system can be introduced systemically, e.g., by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g., Chen et al. (1994) PNAS 91: 3054-3057). An ARDD therapeutic gene can be delivered in a gene therapy construct by electroporation using techniques described, for example, by Dev et al. ((1994) Cancer Treat Rev 20:105-115).

The pharmaceutical preparation of the gene therapy construct or compound of the invention can consist essentially of the gene delivery system in an acceptable diluent, or can comprise a slow release matrix in which the gene delivery vehicle or compound is imbedded. Alternatively, where the complete gene delivery system can be produced intact from recombinant cells, e.g., retroviral vectors, the pharmaceutical preparation can comprise one or more cells which produce the gene delivery system.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

The following examples are illustrative, but not limiting, of the methods and compositions of the present invention. Other suitable modifications and adaptations of the variety of conditions and parameters normally encountered in therapy and that are obvious to those skilled in the art are within the spirit and scope of the embodiments.

### Example 1

### Exemplary Methods for Detection of Certain IL-1 Alleles

Blood is taken by venipuncture and stored uncoagulated at −20° C. prior to DNA extraction. Ten milliliters of blood are added to 40 ml of hypotonic red blood cell (RBC) lysis solution (10 mM Tris, 0.32 Sucrose, 4 mM MgCl2, 1% Triton X-100) and mixed by inversion for 4 minutes at room temperature (RT). Samples are then centrifuged at 1300 g for 15 minutes, the supernatant aspirated and discarded, and another 30 ml of RBC lysis solution added to the cell pellet. Following centrifugation, the pellet is resuspended in 2 ml white blood cell (WBC) lysis solution (0.4 M Tris, 60 mM EDTA, 0.15 M NaCl, 10% SDS) and transferred into a fresh 15 ml polypropylene tube. Sodium perchlorate is added at a final concentration of 1M and the tubes are first inverted on a rotary mixer for 15 minutes at RT, then incubated at 65° C. for 25 minutes, being inverted periodically. After addition of 2 ml of chloroform (stored at −20° C.), samples are mixed for 10 minutes at room temperature and then centrifuged at 800 G for 3 minutes. At this stage, a very clear distinction of phases can be obtained using 300 l Nucleon Silica suspension (Scotlab, UK) and centrifugation at 1400 G for 5 minutes. The resulting aqueous upper layer is transferred to a fresh 15 ml polypropylene tube and cold ethanol (stored at −20° C.) is added to precipitate the DNA. This is spooled out on a glass hook and transferred to a 1.5 ml eppendorf tube containing 500 l TE or sterile water. Following overnight resuspension in TE, genomic DNA yield is calculated by spectrophotometry at 260 nm. Aliquots of samples are diluted at 100 ug/ml, transferred to microtiter containers and stored at 4° C. Stocks are stored at −20° C. for future reference.

Generally, alleles are detected by PCR followed by a restriction digest or hybridization with a probe. Exemplary primer sets and analyses are presented for exemplary loci.

IL-1RN (+2018). PCR primers are designed (mismatched to the genomic sequence) to engineer two enzyme cutting sites on the two alleles to allow for RFLP analysis. The gene accession number is X64532. Oligonucleotide primers are:

| | |
|---|---|
| 5′ CTATCTGAGGAACAACCAACTAGTAGC 3′ | (SEQ ID No. 13) |
| 5′ TAGGACATTGCACCTAGGGTTTGT 3′ | (SEQ ID No. 14) |

Cycling is performed at [96° C., 1 min]; [94° C., 1 min; 57° C., 1 min; 70° C., 2 min;]×35; [70° C., 5 min]×1; 4° C. Each PCR reaction is divided in two 25 ul aliquots: to one is added 5 Units of Alu 1, to the other 5 Units of Msp 1, in addition to 3 ul of the specific 10× restriction buffer. Incubation is at 37° C. overnight. Electrophoresis is by PAGE 9%.

The two enzymes cut respectively the two different alleles. Alu 1 will produce 126 and 28 bp fragments for allele 1, while it does not digest allele 2 (154 bp). Msp 1 will produce 125 and 29 bp with allele 2, while allele 1 is uncut (154 bp). Hence the two reactions (separated side by side in PAGE) will give inverted patterns of digestion for homozygotes, and identical patterns in heterozygotes. Allelic frequencies are 0.74 and 0.26.

IL-1RN (VNTR). The IL1-RN (VNTR) marker may be genotyped in accordance with the following procedure. As indicated above, the two alleles of the IL1-RN (+2018) marker are >97% in linkage disequilibrium with the two most frequent alleles of IL-1RN (VNTR), which are allele 1 and allele 2. The gene accession number is X64532. The oligonucleotide primers used for PCR amplification are:

| | |
|---|---|
| 5′ CTCAGCAACACTCCTAT 3′ | (SEQ ID No. 11) |
| 5′ TCCTGGTCTGCAGGTAA 3′ | (SEQ ID No. 12) |

Cycling is performed at [96° C., 1 min]×1; [94° C., 1 min; 60° C., 1 min; 70° C., 2 min]×35; [70° C., 5 min]×1; 4° C. Electrophoresis is conducted in 2% agarose at 90V for 30 min.

The PCR product sizes are direct indication of number of repeats: the most frequent allele (allele 1) yields a 412 bp product. As the flanking regions extend for 66 bp, the remaining 344 bp imply four 86 bp repeats. Similarly, a 240 bp product indicates 2 repeats (allele 2), 326 is for 3 repeats (allele 3), 498 is 5 (allele 4), 584 is 6 (allele 6). Frequencies for the four most frequent alleles are 0.734, 0.241, 0.021 and 0.004.

IL-1A (−889). The IL-1A (−889) marker may be genotyped in accordance with the following procedure. McDowell et al., Arthritis Rheum. 38:221-28, 1995. One of the PCR primers has a base change to create an Nco I site when amplifying allele 1 (C at −889) to allow for RFLP analysis. The gene accession number is X03833. The oligonucleotide primers used for PCR amplification are:

(SEQ ID No. 17)

5′ AAG CTT GTT CTA CCA CCT GAA CTA GGC 3′

(SEQ ID No. 18)

5′ TTA CAT ATG AGC CTT CCA TG 3′

MgCl₂ is used at 1 mM final concentration, and PCR primers are used at 0.8 μM. Cycling is performed at [96° C., 1 min]×1; [94° C., 1 min; 50° C., 1 min; 72° C., 2 min]×45; [72° C., 5 min]×1; 4° C. To each PCR reaction is added 6 Units of Nco I in addition to 3 μl of the specific 10× restriction buffer. Incubation is at 37/overnight. Electrophoresis is conducted by 6% PAGE. Nco I digest will produce fragments 83 and 16 bp in length, whereas the restriction enzyme does not cut allele 2. Correspondingly, heterozygotes will have three bands. Frequencies for the two alleles are 0.71 and 0.29.

IL-1A (+4845). The IL-1A (+4845) marker may be genotyped in accordance with the following procedure. The PCR primers create an Fnu 4H1 restriction site in allele 1 to allow for RFLP analysis. The gene accession number is X03833. The oligonucleotide primers used for PCR amplification are:

(SEQ ID No. 5)

5′ ATG GTT TTA GAA ATC ATC AAG CCT AGG GCA 3′

(SEQ ID No. 6)

5′ AAT GAA AGG AGG GGA GGA TGA CAG AAA TGT 3′

MgCl₂ is used at 1 mM final concentration, and PCR primers are used at 0.8 μM. DMSO is added at 5% and DNA template is at 150 ng/50 μl PCR. Cycling is performed at [95° C., 1 min]×1; [94° C., 1 min; 56° C., 1 min; 72° C., 2 min]×35; [72° C., 5 min]×1; 4° C. To each PCR reaction is added 2.5 Units of Fnu 4H1 in addition to 2 μl of the specific 10× restriction buffer. Incubation is at 37° C. overnight. Electrophoresis is conducted by 9% PAGE.

Fnu 4H1 digest will produce a constant band of 76 bp (present regardless of the allele), and two further bands of 29 and 124 bp for allele 1, and a single further band of 153 bp for allele 2. Frequencies for the two alleles are 0.71 and 0.29.

IL-1B (−511). The IL-1B (−511) marker may be genotyped in accordance with the following procedure. The gene accession number is X04500. The oligonucleotide primers used for PCR amplification are:

| | |
|---|---|
| 5′ TGG CAT TGA TCT GGT TCA TC 3′ | (SEQ ID No. 7) |
| 5′ GTT TAG GAA TCT TCC CAC TT 3′ | (SEQ ID No. 8) |

MgCl₂ is used at 2.5 mM final concentration, and PCR primers are used at 1 PM. Cycling is performed at [95° C., 1 min]×1; [95° C., 1 min; 53° C., 1 min; 72° C., 1 min]×35; [72° C., 5 min]×1; 4° C. Each PCR reaction is divided into two aliquots: to one aliquot is added 3 Units of Ava I, to the other aliquot is added 3.7 Units of Bsu 36I. To both aliquots is added 3 μl of the specific 10× restriction buffer. Incubation is at 37° C. overnight. Electrophoresis is conducted by 9% PAGE.

Each of the two restriction enzymes cuts one of the two alleles, which allows for RFLP analysis. Ava I will produce two fragments of 190 and 114 bp with allele 1, and it does not cut allele 2 (304 bp). Bsu 361 will produce two fragments of 190 and 11 base pairs with allele 2, and it does not cut allele 1 (304 bp). Frequencies for the two alleles are 0.61 and 0.39.

Procedure for identifying the single base variation (C/T) polymorphism at IL-1B base -511 are described in U.S. Pat. No. 5,686,246 and U.S. Pat. No. 6,140,047, the disclosures of which is incorporated herein by reference in their entireties.

IL-1B (+3954). The IL-1B (+3954) marker may be genotyped in accordance with the following procedure. The gene accession number is X04500. The oligonucleotide primers used for PCR amplification are:

(SEQ ID No. 9)

5′ CTC AGG TGT CCT CGA AGA AAT CAA A 3′

(SEQ ID No. 10)

5′ GCT TTT TTG CTG TGA GTC CCG 3′

MgCl₂ is used at 2.5 mM final concentration, and DNA template at 150 ng/50 μl PCR. Cycling is performed at [95° C., 2 min]×1; [95° C., 1 min; 67.5° C., 1 min; 72° C., 1 min]×35; [72° C., 5 min]×1; 4° C. To each PCR reaction is added 10 Units of Taq I (Promega) in addition to 3 μl of the specific 10× restriction buffer. Incubation is at 65/overnight. Electrophoresis is conducted by 9% PAGE.

The restriction enzyme digest produces a constant band of 12 bp and either two further bands of 85 and 97 bp corresponding to allele 1, or a single band of 182 bp corresponding to allele 2. Frequencies for the two alleles are 0.82 and 0.18.

IL-1B (−3737): Methods for detection of this allele are described in detail in U.S. Patent Publication No. 2003/0235890 to Wyllie et al., the disclosure of which is incorporated herein by reference in its entirety.

### Example 2

Skin inflammatory response to an external stimuli is influenced by the IL-1 genotypic variants. The following example provides evidence that individuals with a hypo-inflammatory genotype require a stronger stimulus to elicit an inflammatory response than subjects with a pro-inflammatory genotype.

In this example, the response from an UV-light stimulus impacting a defined area of skin in serially graded doses is measured by the amount of energy (minimal erythemal dose, MED; given in “seconds of exposure to a calibrated, standardized UV source”) required to elicit a minimal erythemal response (skin reddening). This is a standard test in the dermatologic/cosmetic fields of research. In this study, subjects were screened for selected genotypes representing “genotype-specific groups” (1, 2, 3) prior to determination of their MED (see attached spread sheet for genotypes (IL-1 SNPs) included in each group). The IL-1 allelic patterns for each of Groups I, 2, and 3 are provided in Table 1 below. Briefly, the Group 1 genotype included the allelic pattern of IL-1A (+4845) allele 2, IL-1B (+3954) allele 2, IL-1B (−511) allele 1, IL-1B (−3737) allele 1, and IL-1RN (+2018) allele 1; the Group 2 genotype included the allelic pattern of IL-1A (+4845) allele 1, IL-1B (+3954) allele 1, IL-1B (−511) allele 2, IL-1B (−3737) allele 1, and IL-1RN (+2018) allele 2; and the Group 3 genotype included the allelic pattern of IL-1A (+4845) allele 1, IL-1B (+3954) allele 1, IL-1B (−511) allele 1, IL-1B (−3737) allele 2, and IL-1RN (+2018) allele 1.

Each subject's minimal erythema dose (MED) was determined on the buttocks. Up to seven irradiation exposures were given on adjacent unprotected skin sites. Each exposure represented a 25% increase in energy over the previous exposure. The sites were graded for immediate erythema (IE) and immediate pigment darkening (IPD) after completion of each exposure. The 7 exposures were performed within a space on the buttock cheek of about 7 cm×4 cm. The exposures were made on skin that is even in color and that appears to have received little previous UV exposure.

UV radiation was supplied by an artificial source that has a spectral output in the ultraviolet range comparable to that of the natural solar spectrum. A single port solar simulator with a 150-watt xenon arc lamp (Model 16S, Solar UV Simulator, Solar Light Co., Philadelphia, Pa.) was used for irradiation. UVA+UVB radiation was obtained by using a combination of the UG-5 or UG-11 and WG-320 filters, (Schott Glass Technologies) placed in the radiation path of the solar simulator. The protocol is summarized in the following.

A 1 cm diameter beam of radiation strikes the skin at a distance of 3 inches from the lamp housing. The radiation output of the xenon bulb is measured using the 3D-600 meter (Solar Light Co.). Measurements are taken at least 30 minutes after lamp warm-up. UVA/UVB radiation output are recorded in MED/hr/cm² and in J/cm² prior to MED determination and on each day of irradiation. If necessary, the radiation and spectral output may be adjusted to remain constant throughout the duration of this study. The spectral output of the system is measured and adjusted if necessary at the beginning of each exposure day and documented for the Sponsor. If more than one solar simulator is needed, they are adjusted to provide the same radiation and spectral output. This radiation and spectral output are adjusted to the same values for all simulators and remain constant throughout the duration of this study. The spectral output of the system is measured and adjusted if necessary at the beginning of each exposure day and documented for the Sponsor.

MED Determination/Screening was carried out as follows. Twenty-four (24) hours after MED exposure series, all sites were clinically graded for erythema. The data will be provided as the grade given to each site by the scorer for each of eight sites (one unexposed, seven exposed). Data was sorted by group subject number and UVR dose. The average score for each of exposure sites receiving the same UVR dose was calculated for each group. The average score for sites exposed to the same UVR dose within a group were then compared between groups 1, 2 and 3 using ANOVA with Fisher's LSD. Statistical significance was determined at p≦0.05.

The results indicate that the MED of Group #2 is significantly different from that of either Groups #1 or #3; and this group shows a hypo-inflammatory response pattern because a greater UV exposure (seconds) is required to elicit a minimal erythemal response (MED).

TABLE 1
Genotype Definition of Groups (Skin Pilot Studies)

| | | | | | |
|---|---|---|---|---|---|
| | | | | | Cauc. |
| | | | | | Frequency |
| A(+4845) | B(+3954) | B(−511) | B(−3737) | RN(+2018) | (%) |

Group #1

| | | | | | | | |
|---|---|---|---|---|---|---|---|
| “Pure” | 2.2 | 2.2 | 1.1 | 1.1 | 1.1 | | 2.70 |
| “Accepted” | 2.2 | 1.2 | 1.1 | 1.2 | 1.1 | | 1.00 |
| “Accepted” | 2.2 | 2.2 | 1.1 | 1.2 | 1.1 | | 0.32 |
| “Accepted” | 2.2 | 1.2 | 1.1 | 1.1 | 1.1 | | 0.08 |
| “Accepted” | 1.2 | 2.2 | 1.1 | 1.1 | 1.1 | | 0.80 |
| | | | | | | Tot. % | 4.90 |

Group #2

| | | | | | | | |
|---|---|---|---|---|---|---|---|
| “Pure” | 1.1 | 1.1 | 2.2 | 1.1 | 2.2 | | 2.00 |
| “Accepted” | 1.1 | 1.1 | 2.2 | 1.1 | 1.2 | | 3.60 |
| | | | | | | Tot. % | 5.60% |

Group #3

| | | | | | | | |
|---|---|---|---|---|---|---|---|
| “Pure” | 1.1 | 1.1 | 1.1 | 2.2 | 1.1 | | 9.80 |
| | | | | | | Tot. % | 9.80 |

TABLE 2
Results from MED Determination/Screening

| Genotype Group #1 | | | Genotype Group #2 | | | Genotype Group #3 | | |
|---|---|---|---|---|---|---|---|---|
| | SUB | MED in | | SUB | MED in | | SUB | MED in |
| Count | # | sec | Count | # | sec | Count | # | sec |
| 1 | 031 | 24 | 1 | 053 | 38 | 1 | 008 | 48 |
| 2 | 089 | 38 | 2 | 137 | 48 | 2 | 012 | 38 |
| 3 | 107 | 48 | 3 | 153 | 38 | 3 | 013 | 38 |
| 4 | 110 | 38 | 4 | 219 | 60 | 4 | 037 | 38 |
| 5 | 144 | 48 | 5 | 229 | 48 | 5 | 038 | 48 |
| 6 | 224 | 30 | 6 | 233 | 48 | 6 | 044 | 38 |
| 7 | 271 | 48 | 7 | 314 | 75 | 7 | 048 | 75 |
| 8 | 383 | 48 | 8 | 315 | 38 | 8 | 058 | 30 |
| 9 | 411 | 48 | 9 | 346 | 38 | 9 | 062 | 30 |
| 10 | 439 | 38 | 10 | 379 | 60 | 10 | 069 | 38 |
| 11 | 457 | 48 | 11 | 419 | 38 | 11 | 074 | 30 |
| 12 | 473 | 38 | 12 | 425 | 60 | 12 | 078 | 30 |
| 13 | 560 | 48 | 13 | 437 | 48 | 13 | 106 | 38 |
| 14 | 573 | 38 | 14 | 440 | 48 | 14 | 136 | 48 |
| 15 | 595 | 38 | 15 | 511 | 75 | 15 | 162 | 38 |
| | Avg | 41.2 | 16 | 572 | 38 | 16 | 201 | 30 |
| | Std | 7.59 | 17 | 606 | 30 | 17 | 293 | 48 |
| | Min | 24 | | Avg | 48.7 | 18 | 322 | 38 |
| | Max | 48 | | Std | 13.2 | 19 | 323 | 48 |
| | | | | Min | 30 | 20 | 335 | 38 |
| | | | | Max | 75 | 21 | 381 | 38 |
| | | | | | | 22 | 430 | 60 |
| | | | | | | 23 | 444 | 38 |
| | | | | | | 24 | 467 | 38 |
| | | | | | | 25 | 497 | 38 |
| | | | | | | 26 | 518 | 30 |
| | | | | | | 27 | 520 | 38 |
| | | | | | | 28 | 544 | 38 |
| | | | | | | 29 | 546 | 48 |
| | | | | | | 30 | 601 | 30 |
| | | | | | | 31 | 618 | 38 |
| | | | | | | | Avg | 40.0 |
| | | | | | | | Std | 9.6 |
| | | | | | | | Min | 30 |
| | | | | | | | Max | 75 |

Statistical Analysis:
Grp #2 vs Grp #3- p(one-tail) = 0.01
p(two-tailed) = 0.03
Grp #2 vs Grp #1- p(one-tailed) = 0.03
p(two-tailed) = 0.06

### Example 3

Example 2 identifies differences in response to UV-Light (Solar Simulator) between young females who were placed into one of three genotype groups based on IL-1 polymorphism genotyping. Their response (as measured by MED) indicated that women in Group #2 required a higher energy dosage to produce a minimal visible erythemic (inflammatory) response than women in either Groups #1 or #3.

In addition to the analysis of associations between MED and subject groups defined by specific genotypes, an analysis was performed on single SNP associations with quantitative measures of baseline skin color (expressed as L* and a* values as explained below) and response to UV-light (MED). The skin type of individuals (as measured by the Fitzpatrick skin type scale) was also tested as a confounding variable in these SNP associations with response to UV-light.

As used in this Example, Minimal Erythemal Dose (MED) is an indication of how much exposure to UV light is required to induce the first signs of skin reddening. The higher the value, the more energy needed to redden the skin. Skin with a higher MED value is therefore the least inflammatory after UV. The lower the value the more inflammatory response to UV in that skin.

The Average L* value is the measure of skin color; the higher the value—the lighter the color (pigmentation); the lower the value—the darker the pigmentation. The Average L* value may be used as an adjustment factor, since higher pigmentation (lower L* value) may protect skin from UV light. It would be expected that hyper-inflammatory genotypes may associate with higher skin pigment levels (Skin darkness).

The Average a* value is the measure of skin color. The higher the value the redder the skin color (measure of erythema). It would expected that hyper-inflammatory IL-1 genotypes would have higher values (redness) even without UV exposure.

As is shown in Table 3 below, no associations were found between the IL-1B −511 polymorphism and L* or a* variables. However, an interesting association between the IL-1B −511 polymorphism and MED was found. Subject with the 1,1 (or 11) genotype (CC) have statistically lower (38.76) values of MED than subjects with the 2,2 (or (22) genotype (45.15); P=0.004. There are no MED determinations for 1,2 (or 12) of the subjects.

The 2.2 (C/C) subjects for the IL-1B −511 polymorphism had a lower inflammatory response in the skin after UV. The 1.1 (T/T) subjects for the IL-1B −511 polymorphism had a higher inflammatory response to UV.

TABLE 3
Associations between the IL-1B-511 polymorphism
and L* or a* variables.

| | | | | | ANOVA | P |
|---|---|---|---|---|---|---|
| | | N | Mean | SD | P | trend |
| L_value | 1,1) C_T_511 | 243 | 67.01 | 4.068 | .409 | |
| | 1,2 | 306 | 66.87 | 4.025 | | .183 |
| | 2,2 | 72 | 66.27 | 4.794 | | |
| | Total | 621 | 66.86 | 4.136 | | |
| A_value | 1,1) C_T_511 | 243 | 7.73 | 1.882 | .452 | |
| | 1,2 | 306 | 7.75 | 1.750 | | .224 |
| | 2,2 | 72 | 8.03 | 2.001 | | |
| | Total | 621 | 7.78 | 1.832 | | |
| MED | 1,1) C_T_511 | 50 | 38.76 | 6.808 | .004 | |
| | 1,2 | 0 | . . . | . . . | | |
| | 2,2 | 20 | 45.15 | 10.459 | | |
| | Total | 70 | 40.59 | 8.455 | | |

As is shown in the table below, no associations were found between the IL-1RN +2018 polymorphism, and L* or a* variables. However, an association between this polymorphism and MED was found. Subjects with the 1,1 genotype (CC) have statistically lower (38.76) values of MED than the other subjects. ANOVA P: 0.005.

However, no statistically significant trend among genotypes was found. A surprisingly higher MED value ((47.90) for 1,2 subjects was found. The 1,1 (TT) subjects for the IL-RN +2018 polymorphism had a higher inflammatory response in the skin after UV. The carriers of the 2 allele of the IL-RN +2018 polymorphism, had higher MED values and then a lower inflammatory response to UV.

TABLE 4
Associations between the IL-1 RN +2018
polymorphism and L* or a* variables.

| | | | | | ANOVA | P |
|---|---|---|---|---|---|---|
| | | N | Mean | SD | P | trend |
| L_value | 1,1) T_C2018 | 328 | 67.06 | 4.153 | .497 | |
| | 1,2 | 247 | 66.65 | 3.963 | | .650 |
| | 2,2 | 53 | 66.78 | 4.828 | | |
| | Total | 628 | 66.87 | 4.139 | | |
| A_value | 1,1) T_C2018 | 328 | 7.67 | 1.835 | .234 | |
| | 1,2 | 247 | 7.92 | 1.725 | | .972 |
| | 2,2 | 53 | 7.68 | 2.229 | | |
| | Total | 628 | 7.77 | 1.830 | | |
| MED | 1,1) T_C2018 | 50 | 38.76 | 6.808 | .005 | |
| | 1,2 | 10 | 47.90 | 11.893 | | .189 |
| | 2,2 | 10 | 42.40 | 8.527 | | |
| | Total | 70 | 40.59 | 8.455 | | |

Even after the additional adjustment for L value, the association between the −511 C>T polymorphism and MED levels remained statistically significant (P=0.025), demonstrating the independence of the association. Further adjustment of this association for the Fitzpatrick skin type did not modified the statistical significance of the −511C>T polymorphism (P<0.05).

### Example 4

Haplotype Influence on Skin Inflammatory Response. The genotypic definition of Group #2 was based on the haplotype (single chromosomal alignment of IL-1 polymorphism alleles) shown in Table 5 (below) as “B2” with the additional stipulation that the IL1RN (+2018) allele would be the rarer variant (“2” or nucleotide “C”). More specifically, Group #2 was defined to include specific haplotype pairs (both chromosomes; or genotype) shown below in Table 6 as B2/B2 and B2/B4. In a similar manner, Group #1 individuals were defined by haplotype B3 and predominantly the B3/B3 haplotype pair, with the stipulation that the IL-1RN (+2018) alleles were 1.1. Group #3 was defined by the B1 haplotype and the B1/B1 haplotype pair, with the stipulation that the IL-1RN (+2018) alleles were 1.1.

TABLE 5
Predominant haplotypes for IL-1 B-promoter and most common
IL-1A (+4845)-IL-1B (+3954)-IL-1B(+3877) haplotypes
found with each IL-1B promoter haplotype.

| B-promoter | IL-1A | IL-1B | IL-1 B | IL1B | IL1B | IL1B |
|---|---|---|---|---|---|---|
| Haplotype | (+4845) | (+3954) | (+3877) | (−511) | (−1464) | (−3737) |
| B1 | 1 | 1 | 2 | 1 | 1 | 2 |
| B2 | 1 | 1 | 1 | 2 | 2 | 1 |
| B3 | 2 | 2 | 1 | 1 | 1 | 1 |
| B4 | 1 | 1 | 1 | 2 | 1 | 1 |

TABLE 6
Predominant IL-1 Haplotype Pairs

| Haplotype | IL-1A | IL-1B | IL-1 B | IL1B | IL1B | IL1B |
|---|---|---|---|---|---|---|
| pairs | (+4845) | (+3954) | (+3877) | (−511) | (−1464) | (−3737) |
| B1/B1 | 1.1 | 1.1 | 2.2 | 1.1 | 1.1 | 2.2 |
| B1/B2 | 1.1 | 1.1 | 1.2 | 1.2 | 1.2 | 1.2 |
| B1/B3 | 1.2 | 1.2 | 1.2 | 1.1 | 1.1 | 1.2 |
| B1/B4 | 1.1 | 1.1 | 1.2 | 1.2 | 1.1 | 1.2 |
| B2/B2 | 1.1 | 1.1 | 1.1 | 2.2 | 2.2 | 1.1 |
| B2/B3 | 1.2 | 1.2 | 1.1 | 1.2 | 1.2 | 1.1 |
| B2/B4 | 1.1 | 1.1 | 1.1 | 2.2 | 1.2 | 1.1 |
| B3/B3 | 2.2 | 2.2 | 1.1 | 1.1 | 1.1 | 1.1 |
| B3/B4 | 1.2 | 1.2 | 1.1 | 1.2 | 1.1 | 1.1 |
| B4/B4 | 1.1 | 1.1 | 1.1 | 2.2 | 1.1 | 1.1 |

### Example 5

To demonstrate the functional significance of these IL-1 promoter haplotypes, a separate study was performed on peripheral blood mononucleated cells from individuals of known IL-1 genotype (Caucasian; Italian). The amount of IL-1B protein released by such cells was measured in vitro before and after stimulation by endotoxin (LPS). The haplotype pairs which have shown the major differential in skin inflammatory response to stimulation (UV-light) are also the pairs that demonstrated major difference in production of IL-1B protein in this model system (Table 7 below).

TABLE 7
Mean changes in IL1B levels after stimulation.
according to IL-1 beta promoter haplotype pair

| | |
|---|---|
| IL1B promoter | (IL-1B stim minus IL-1B baseline) |

| Haplotype pairs | N | Geometric mean* | SD | P value** |
|---|---|---|---|---|
| B1B1 | 20 | 2.51 | 3.18 | <0.0001 |
| B1B2 | 12 | 1.18 | 2.49 | 0.0005 |
| B1B3 | 10 | 1.56 | 4.07 | 0.002 |
| B1B4 | 5 | 1.47 | 3.43 | 0.0625 |
| B2B2 | 4 | 0.36 | 4.62 | 0.125 |
| B2B3 | 4 | 2.61 | 2.05 | 0.125 |
| B2B4 | 7 | 0.55 | 1.8 | 0.0156 |
| B3B3 | 4 | 3.25 | 2.11 | 0.125 |
| B3B4 | 3 | 2.06 | 2.96 | 0.25 |
| B4B4 | 1 | 0.56 | | |

These results indicate that the genetic differences seen between genotype groups in the skin study, with response to an external inducer of inflammation (UV-Light) are particularly relevant because of their discrimination between predominant haplotypes found in not only the Caucasian population (as tested), but in other world-wide populations when focusing on predominant haplotypes as seen in Table 6 above.

Genotype Definitions:

| | | |
|---|---|---|
| IL1 [symbol] (+3954) | C/C | 1.1 |
| | C/T | 1.2 |
| | T/T | 2.2 |
| IL1 [symbol] (−511) | C/C | 1.1 |
| | C/T | 1.2 |
| | T/T | 2.2 |
| ILIA (+4845) | G/G | 1.1 |
| | G/T | 1.2 |
| | T/T | 2.2 |
| IL1B (−3737) | C/C | 1.1 |
| | C/T | 1.2 |
| | T/T | 2.2 |
| IL1B (+3877) | G/G | 1.1 |
| | A/G | 1.2 |
| | A/A | 2.2 |
| IL1RN_rs315952 | T/T | 1.1 |
| | C/T | 1.2 |
| | C/C | 2.2 |
| IL1RN_rs9005 | G/G | 1.1 |
| | A/G | 1.2 |
| | A/A | 2.2 |
| IL1RN + (2018) | T/T | 1.1 |
| | C/T | 1.2 |
| | CC | 2.2 |

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

From the foregoing detailed description of the specific embodiments of the invention, it should be apparent that unique methods have been described. Although particular embodiments have been disclosed herein in detail, this has been done by way of example for purposes of illustration only, and is not intended to be limiting with respect to the scope of the appended claims which follow. In particular, it is contemplated by the inventor that various substitutions, alterations, and modifications may be made to the invention without departing from the spirit and scope of the invention as defined by the claims.

While the invention has been described with reference to particularly preferred embodiments and examples, those skilled in the art recognize that various modifications may be made to the invention without departing from the spirit and scope thereof.

All of the above U.S. patents, U.S. patent application publications, U.S. patent applications, foreign patents, foreign patent applications and non-patent publications referred to in this specification and/or listed in the Application Data Sheet are incorporated herein by reference, in their entirety.

What is claimed is:

1. A method of predicting a subject's propensity to developing a dermatologic disorder, comprising the steps of:
(a) isolating genomic DNA from a patient;
(b) determining a genetic polymorphism pattern for IL-1B and IL-1RN in the genomic DNA; and
(c) comparing the genetic polymorphism patterns to a control sample, wherein said control sample comprises an IL-1RN (+2018) allele 2 and IL-1B (−511) allele 2, and wherein the similarity of the genetic polymorphism pattern to the control sample indicates reduced susceptibility to developing a dermatologic disorder.

2. The method as set forth in claim 1 wherein the control samples are ethnically matched control samples.

3. The method as set forth in claim 1, wherein the method step of determining a genetic polymorphism pattern for IL-1B and IL-1RN further comprises detecting at least one allele in linkage disequilibrium with IL-1B (−511) allele 2 and IL-1RN (+2018) allele 2.

4. The method as set forth in claim 1, wherein the method step of determining a genetic polymorphism pattern for IL-1B and IL-1RN further comprises detecting at least one allele selected from the group consisting of IL-1A (+4845) allele 1, IL-1B (+3954) allele 1, and IL-1B (−3737) allele 1.

5. A method of predicting a subject's propensity to developing a dermatologic disorder, said method comprising the steps of:
a) isolating genomic DNA from a patient; and
b) determining an allelic pattern for IL-1B and IL-1RN in the genomic DNA;
wherein the allelic pattern of at least one copy of IL-1RN (+2018) allele 2 and at least one copy of IL-1B (−511) allele 2 indicates decreased susceptibility to developing an inflammatory-based dermatologic disorder.

6. The method as in claim 5, wherein said step of determining an allelic pattern comprises amplification with a polymerase chain reaction (PCR) and at least one PCR primer, wherein said PCR primer is selected from the group consisting of: 5′ CTCAGCAACACTCCTAT 3′ (SEQ ID No. 11); 5′ TCCTGGTCTGCAGGTAA 3′ (SEQ ID No. 12), 5′ TGG CAT TGA TCT GGT TCA TC 3′ (SEQ ID No. 7); and 5′ GTT TAG GAA TCT TCC CAC TT 3′ (SEQ ID No. 8).

7. The method as in claim 5, wherein said step of determining an allelic pattern comprises digestion with at least one restriction enzyme selected from the group consisting of AvaI and Bsu36I.

8. The method of claim 5 further comprising determining the presence of at least one additional allele found in a predominant haplotype with IL-1B (−511) allele 2 or IL-1RN (+2018) allele 2 in the genomic DNA, wherein the at least one additional allele is selected from the group consisting of allele 1 of IL-1A (−3737), allele 1 of IL-1B (+3954), allele 1 of IL-1B (+3877), allele 2 of IL-1B (−1464), and allele 1 of IL-1B (−3737).

9. The method of claim 5 further comprising determining the presence of at least one allelic pair found in a predominant haplotype pair with IL-1B (−511) allele 2 or IL-1RN (+2018) allele 2 in the genomic DNA, wherein the at least one allelic pair is selected from the group consisting of: allele 1 of IL-1A (−3737) and allele 1 of IL-1A (−3737); allele 1 of IL-1B (+3954) and allele 1 of IL-1B (+3954); allele 1 of IL-1B (+3877) and allele 1 of IL-1B (+3877); allele 1 of IL-1B (−1464) and allele 2 of IL-1B (−1464); and allele 1 of IL-1B (−3737) and allele 1 of IL-1B (−3737).

10. The method of claim 5 further comprising determining the presence of at least one allelic pair found in a predominant haplotype pair with IL-1B (−511) allele 2 or IL-1RN (+2018) allele 2 in the genomic DNA, wherein the at least one allelic pair is selected from the group consisting of: allele 1 of IL-1A (−3737) and allele 1 of IL-1A (−3737); allele 1 of IL-1B (+3954) and allele 1 of IL-1B (+3954); allele 1 of IL-1B (+3877) and allele 1 of IL-1B (+3877); allele 2 of IL-1B (−1464) and allele 2 of IL-1B (−1464); and allele 1 of IL-1B (−3737) and allele 1 of IL-1B (−3737).

11. The method of claim 5 wherein the allelic pattern that indicates decreased susceptibility to developing an inflammatory-based dermatologic disorder includes an IL-1 polymorphic allele found to be in linkage disequilibrium with an IL-1 inflammatory haplotype, wherein the IL-1 inflammatory haplotype comprises the alleles selected from the group consisting of allele 1 of IL-1A (−3737), allele 1 of IL-1B (+3954), allele 1 of IL-1B (+3877), allele 2 of IL-1B (−1464), and allele 1 of IL-1B (−3737).

12. The method of claim 5 wherein the allelic pattern that indicates decreased susceptibility to developing an inflammatory-based dermatologic disorder includes an IL-1 polymorphic allele found to be in linkage disequilibrium with an IL-1 inflammatory haplotype pair, wherein the IL-1 inflammatory haplotype pair comprises the allelic pair selected from the group consisting of: allele 1 of IL-1A (−3737) and allele 1 of IL-1A (−3737); allele 1 of IL-1B (+3954) and allele 1 of IL-1B (+3954); allele 1 of IL-1B (+3877) and allele 1 of IL-1B (+3877); allele 2 of IL-1B (−1464) and allele 2 of IL-1B (−1464); and allele 1 of IL-1B (−3737) and allele 1 of IL-1B (−3737).

13. The method of claim 5 wherein the allelic pattern that indicates decreased susceptibility to developing an inflammatory-based dermatologic disorder includes an IL-1 polymorphic allele found to be in linkage disequilibrium with an IL-1 inflammatory haplotype pair, wherein the IL-1 inflammatory haplotype pair comprises the allelic pair selected from the group consisting of: allele 1 of IL-1A (−3737) and allele 1 of IL-1A (−3737); allele 1 of IL-1B (+3954) and allele 1 of IL-1B (+3954); allele 1 of IL-1B (+3877) and allele 1 of IL-1B (+3877); allele 1 of IL-1B (−1464) and allele 2 of IL-1B (−1464); and allele 1 of IL-1B (−3737) and allele 1 of IL-1B (−3737).

14. A kit for predicting a patient's susceptibility to a dermatologic disorder, said kit comprising:
(a) a DNA sample collecting means;
(b) a means for determining a genetic polymorphism pattern for IL-1B and IL-1RN , wherein said means comprises a set of polymerase chain reaction (PCR) primers, and
(c) a control sample comprising IL-1RN (+2018) allele 2 and IL-1B (−511) allele 2.

15. A method of predicting a subject's propensity to developing a dermatologic disorder, comprising the steps of:
(a) isolating genomic DNA from a patient;
(b) determining a genetic polymorphism pattern for IL-1B and IL-1RN in the genomic DNA; and
(c) comparing the genetic polymorphism patterns to a control sample, wherein said control sample comprises an IL-1RN (+2018) allele 1 and IL-1B (−511) allele 1, and wherein the similarity of the genetic polymorphism pattern to the control sample indicates increased susceptibility to developing a dermatologic disorder.

16. The method as set forth in claim 1 wherein the control samples are ethnically matched control samples.

17. The method as set forth in claim 1, wherein the method step of determining a genetic polymorphism pattern for IL-1B and IL-1RN further comprises detecting at least one allele in linkage disequilibrium with IL-1B (−511) allele 1 and IL-1RN (+2018) allele 1.

18. The method as set forth in claim 1, wherein the method step of determining a genetic polymorphism pattern for IL-1B and IL-1RN further comprises detecting at least one allele selected from the group consisting of IL-1A (+4845) allele 2, IL-1B (+3954) allele 2, and IL-1B (−3737) allele 1.

19. The method as set forth in claim 1, wherein the method step of determining a genetic polymorphism pattern for IL-1B and IL-1RN further comprises detecting at least one allele selected from the group consisting of IL-1A (+4845) allele 1, IL-1B (+3954) allele 1, and IL-1B (−3737) allele 2.


SEQUENCE LISTING (23 sequences)

SEQ ID NO: 1 - DNA, 11970 bp, Homo sapiens
aagcttctaccctagtctggtgctacacttacattgcttacatccaagtgtggttatttctgtggctcctgttataactattatagcaccaggtctatgaccaggagaattagactggcattaaatcagaataagagattttgcacctgcaatagaccttatgacacctaaccaaccccattatttacaattaaacaggaacagagggaatactttatccaactcacacaagctgttttcctcccagatccatgcttttttgcgtttattattttttagagatgggggcttcactatgttgcccacactggactaaaactctgggcctcaagtgattgtcctgcctcagcctcctgaatagctgggactacaggggcatgccatcacacctagttcatttcctctatttaaaatatacatggcttaaactccaactgggaacccaaaacattcatttgctaagagtctggtgttctaccacctgaactaggctggccacaggaattataaaagctgagaaattctttaataatagtaaccaggcaacatcattgaaggctcatatgtaaaaatccatgccttcctttctcccaatctccattcccaaacttagccactggttctggctgaggccttacgcatacctcccggggcttgcacacaccttcttctacagaagacacaccttgggcatatcctacagaagaccaggcttctctctggtccttggtagagggctactttactgtaacagggccagggtggagagttctctcctgaagctccatcccctctataggaaatgtgttgacaatattcagaagagtaagaggatcaagacttctttgtgctcaaataccactgttctcttctctaccctgccctaaccaggagcttgtcaccccaaactctgaggtgatttatgccttaatcaagcaaacttccctcttcagaaaagatggctcattttccctcaaaagttgccaggagctgccaagtattctgccaattcaccctggagcacaatcaacaaattcagccagaacacaactacagctactattagaactattattattaataaattcctctccaaatctagccccttgacttcggatttcacgatttctcccttcctcctagaaacttgataagtttcccgcgcttccctttttctaagactacatgtttgtcatcttataaagcaaaggggtgaataaatgaaccaaatcaataacttctggaatatctgcaaacaacaataatatcagctatgccatctttcactattttagccagtatcgagttgaatgaacatagaaaaatacaaaactgaattcttccctgtaaattccccgttttgacgacgcacttgtagccacgtagccacgcctacttaagacaattacaaaaggcgaagaagactgactcaggcttaagctgccagccagagagggagtcatttcattggcgtttgagtcagcaaaggtattgtcctcacatctctggctattaaagtattttctgttgttgtttttctctttggctgttttctctcacattgccttctctaaagctacagtctctcctttcttttcttgtccctccctggtttggtatgtgacctagaattacagtcagatttcagaaaatgattctctcattttgctgataaggactgattcgttttactgagggacggcagaactagtttcctatgagggcatgggtgaatacaactgaggcttctcatgggagggaatctctactatccaaaattattaggagaaaattgaaaatttccaactctgtctctctcttacctctgtgtaaggcaaataccttattcttgtggtgtttttgtaacctcttcaaactttcattgattgaatgcctgttctggcaatacattaggttgggcacataaggaataccaacataaataaaacattctaaaagaagtttacgatctaataaaggagacaggtacatagcaaactaattcaaaggagctagaagatggagaaaatgctgaatgtggactaagtcattcaacaaagttttcaggaagcacaaagaggaggggctcccctcacagatatctggattagaggctggctgagctgatggtggctggtgttctctgttgcagaagtcaagatggccaaagttccagacatgtttgaagacctgaagaactgttacaggtaaggaataagatttatctcttgtgatttaatgagggtttcaaggctcaccagaatccagctaggcataacagtggccagcatgggggcaggccggcagaggttgtagagatgtgtactagtcctgaagtcagagcaggttcagagaagacccagaaaaactaagcattcagcatgttaaactgagattacattggcagggagaccgccattttagaaaaattatttttgaggtctgctgagccctacatgaatatcagcatcaacttagacacagcctctgttgagatcacatgccctgatataagaatgggttttactggtccattctcaggaaaacttgatctcattcaggaacaggaaatggctccacagcaagctgggcatgtgaactcacatatgcaggcaaatctcactcagatgtagaagaaaggtaaatgaacacaaagataaaattacggaacatattaaactaacatgatgtttccattatctgtagtaaatactaacacaaactaggctgtcaaaattttgcctggatattttactaagtataaattatgaaatctgttttagtgaatacatgaaagtaatgtgtaacatataatctatttggttaaaataaaaaggaagtgcttcaaaacctttcttttctctaaaggagcttaacattcttccctgaacttcaattaaagctcttcaatttgttagccaagtccaatttttacagataaagcacaggtaaagctcaaagcctgtcttgatgactactaattccagattagtaagatatgaattactctacctatgtgtatgtgtagaagtccttaaatttcaaagatgacagtaatggccatgtgtatgtgtgtgacccacaactatcatggtcattaaagtacattggccagagaccacatgaaataacaacaattacattctcatcatcttattttgacagtgaaaatgaagaagacagttcctccattgatcatctgtctctgaatcaggtaagcaaatgactgtaattctcatgggactgctattcttacacagtggtttcttcatccaaagagaacagcaatgacttgaatcttaaatacttttgttttaccctcactagagatccagagacctgtctttcattataagtgagaccagctgcctctctaaactaatagttgatgtgcattggcttctcccagaacagagcagaactatcccaaatccctgagaactggagtctcctggggcaggcttcatcaggatgttagttatgccatcctgagaaagccccgcaggccgcttcaccaggtgtctgtctcctaacgtgatgtgttgtggttgtcttctctgacaccagcatcagaggttagagaaagtctccaaacatgaagctgagagagaggaagcaagccagctgaaagtgagaagtctacagccactcatcaatctgtgttattgtgtttggagaccacaaatagacactataagtactgcctagtatgtcttcagtactggctttaaaagctgtccccaaaggagtatttctaaaatattttgagcattgttaagcagatttttaacctcctgagagggaactaattggaaagctaccactcactacaatcattgttaacctatttagttacaacatctcatttttgagcatgcaaataaatgaaaaagtcttcctaaaaaaatcatctttttatcctggaaggaggaaggaaggtgagacaaaagggagagagggagggaagcctaatgaaacaccagttacctaagaccagaatggagatcctcctcactacctctgttgaatacagcacctactgaaagaactttcattccctgaccatgaacagcctctcagcttctgttttccttcctcacagaaatccttctatcatgtaagctatggcccactccatgaaggctgcatggatcaatctgtgtctctgagtatctctgaaacctctaaaacatccaagcttaccttcaaggagagcatggtggtagtagcaaccaacgggaaggttctgaagaagagacggttgagtttaagccaatccatcactgatgatgacctggaggccatcgccaatgactcagaggaaggtaaggggtcaagcacaataatatctttcttttacagttttaagcaagtagggacagtagaatttaggggaaaattaaacgtggagtcagaataacaagaagacaaccaagcattagtctggtaactatacagaggaaaattaatttttatccttctccaggagggagaaatgagcagtggcctgaatcgagaatacttgctcacagccattatttcttagccatattgtaaaggtcgtgtgacttttagcctttcaggagaaagcagtaataagaccacttacgagctatgttcctctcatactaactatgcctccttggtcatgttacataatcttttcgtgattcagtttcctctactgtaaaatggagataatcagaatcccccactcattggattgttgtaaagattaagagtctcaggctttacagactgagctagctgggccctcctgactgttataaagattaaatgagtcaacatcccctaacttctggactagaataatgtctggtacaaagtaagcacccaataaatgttagctattactatcattattattattattttattttttttttttgagatggagtctggctctgtcacccaggctggagtgcagtggcacaatctcggctcactgcaagctctgcctcctgggttcatgccattctcctgcctcagcctcccgagtaagctgggaatacaggcacccgccactgttcccggctaattttttgtatttttagtagagacggagtttcaccgtggtctccatctcctcgtgatccacccaccttggcctcccaaagtgccgggattacaggcgtgagccaccgcgcccggcctattattattattattactactactactacctatatgaatactaccagcaatactaatttattaatgactggattatgtctaaacctcacaagaatcctaccttctcattttacataaaaggaaactaagctcattgagataggtaaactgcccaatggcatacatctgtaagtgggagagcctcaaatctaattcagttctacctgagtaaaaaaatcatggtttctcctccatccctttactgtacaagcctccacatgaactataaacccaatattcctgtttttaagataatacctaagcaataacgcatgttcacctagaaggttttaaaatgtaacaaaatataagaaaataaaaatcactcatatcgtcagtgagagtttactactgccagcactatggtatgtttccttaaaatctttgctatacacatacctacatgtgaacaaatatgtctaacatcaagaccacactatttacaactttatatccagcttttcttacttagcaatgtattgaggacattttagagtgcccgtttttcaccattataagcaatgcaacaatgaacatctgtataaataaatattcatttctctcaccctttatttccttagaatatattcctagaagtagaatttcccagagccatgaggatttgtgacgctattgatatgtgccactttgcactctctgtgacatatataattatttttaatgcattcatttttttctcagagtgcattcgtttgaaaacatagacgggaaatactggtagtcttccttgtcagttagaaacacccaaacaatgaaaaatgaaaaagttgcacaaatagtctctaaaaacaatgaaactattgcctgaggaattgaagtttaaaaagaagcacataagcaacaacaaggataatcctagaaaaccagttctgctgactgggtgatttcacttctctttgcttcctcatctggattggaatattcctaataccccctccagaactattttccctgtttgtactagactgtgtatatcatctgtgtttgtacatagacattaatctgcacttgtgatcatggttttagaaatcatcaagcctaggtcatcaccttttagcttcctgagcaatgtgaaatacaactttatgaggatcatcaaatacgaattcatcctgaatgacgccctcaatcaaagtataattcgagccaatgatcagtacctcacggctgctgcattacataatctggatgaagcaggtacattaaaatggcaccagacatttctgtcatcctcccctcctttcatttacttatttatttatttcaatctttctgcttgcaaaaaacatacctcttcagagttctgggttgcacaattcttccagaatagcttgaagcacagcacccccataaaaatcccaagccagggcagaaggttcaactaaatctggaagttccacaagagagaagtttcctatctttgagagtaaagggttgtgcacaaagctagctgatgtactacctctttggttctttcagacattcttaccctcaattttaaaactgaggaaactgtcagacatattaaatgatttactcagatttacccagaagccaatgaagaacaatcactctcctttaaaaagtctgttgatcaaactcacaagtaacaccaaaccaggaagatctttattatctctgataacatatttgtgaggcaaaacctccaataagctacaaatatggcttaaaggatgaagtttagtgtccaaaaacttttatcacacacatccaattttcatggcggacatgttttagtttcaacagtatacatattttcaaaggtccagagaggcaattttgcaataaacaagcaagactttttctgattggatgcacttcagctaacatgctttcaactctacatttacaaattattttgtgttctatttttctacttaatattatttctgcaattttcccaatattgacatcgtgtatgtatttgccatttttaatatcactagacaattcaatcaggttgctacgttggtcccttgggtttactctaaatagcttgattgcaaatatctttgtatatattattgttttttctcctatcttgtaatttctttgagcacatcccaaagaggaatgcctagatcaatgggcacaaataatttgacagctcttattaaacattattctgtaagtaaaaactgaactacttttcagtatcactagcaacatatgagtgtatcagcttcctaaacccctccatgttaggtcattatgaacttatgatctaacaaattacagggtcttatcccactaatgaaattataagagattcaacacttattcagccccgaaggattcattcaacgtagaaaattctaagaacattaaccaagtatttacctgcctagtgagtgtggaagacattgtgaaggacacaaagatgtatagaattccattcctgacttccaggtatttacaccataggtggggacctaactacacacacacacacacacacacacacacacacacacaccatgcacacacaatctacatcaacacttgattttatacaaatacaatgaatttactttctttttggttcttctcttcaccagtgaaatttgacatgggtgcttataagtcatcaaaggatgatgctaaaattaccgtgattctaagaatctcaaaaactcaattgtatgtgactgcccaagatgaagaccaaccagtgctgctgaaggtcagttgtcctttgtctccaacttaccttcatttacatctcatatgtttgtaaataagcccaataggcagacacctctaacaaggtgacactgtcctctttccttcctaccacagcccccacctacccaccccactcccattgattccagaggcgtgcctaggcaggatctatgagaaaatataacagagagtaagaggaaaattaccttctttctttttcctttccctgcctgaccttattcacctcccatcccagagcatccatttattccattgatctttactgacatctattatctgacctacacaatactagacattaggacaatgtggcctgcctccaagaaactcaaataagccaactgagatcagagaggattaatcacctgccaatgggcacaaagcaacaagctgggagccaagtcccaaaatggggcctgctgcttccagttcccctctctctgcattgatgtcagcattatccttcgtcccagtcctgtctccactaccactttccccctcaaacacacacacacacaacagccttagatgttttctccactgataagtaggtgactcaatttgtaagtatataatccaagaccttctattcccaagtagaatttatgtgcctgcctgtgcttttctacctggatcaagtgatgtctacagagtagggcagtagcttcattcatgaactcattcaacaagcattattcactgagagccttgtatttttcaggcatagtgccaacagcagtgtggacagtggtgcatcaaagcctctagtctcatagaacttagtcttctggaggatatggaaaacagacaacccaaacaaccaacaaaagagcaagatgctgcaaaaaaaaaaaaaatgaatagggtgctaagatagagaaaagtgggagagtgctatttagacaaagtggtaaaaacaaagccccttgtgagatgagagctgccgacagagggggcgggtcatggttgtgggtttttgggtaggacattcagaggagggggcgggtcgtggttgtgggtttttgggtaggacattcagaggagggggcgggtcgtggttgtgggtttttgggtaggacattcagaggagggggcgggtcgtggttgtgggtttttgggtaggacattcagaggagggggcgggtcgtggttgtgggtttttgggacattcagaggagtctgaatgcacccaggcctacaacttcaagatggtaaaggacagctccaaggatcagaagaagcattcttggaactggggcattttgagaaggaggaaaaatatgcagagactagtgcttgcagagcttgcatttggatttcatttgaggtacaatgaaaacccattaatgggtttcacacagtgcaatggcctgacctcacttatatttcctaaaatagaaaacagatcagaaggaaggcaatagagaagcagaaagtccaatgaggaggtttcacagcagtcatgggggtggggtaaggaaaagaagtggaaagaaacagacagaattgggttatattttggagatagaaccaacagaaggaagaggagaaacaacatttactgagaagggaaaaagtaggagaggaataggtttgggaaataaatcctgctgacattggaaaccccaaggaagcctcaaaagtatatttacttgctttagatttaaaagaataggaaagaagcatctcaacttggaatttgaaatctatttttccataaaagtattgttaaattctactcatactcacaagaaaagtacattctaaagagtatattgaaagagtttactgatatacttaggaattttgtgtgtatgtgtgtgtgtgtatgtgtgtgtgtgtgtttaaccttcaattgttgacttaaatactgagataaatgtcatctaaatgctaaattgatttcccaaaggtatgatttgttcacttggagatcaaaatgtttagggggcttagaatcactgtagtgctcagatttgatgcaaaatgtcttaggcctatgttgaaggcaggacagaaacaatgtttccctcctacctgcctggatacagtaagatactagtgtcactgacaatcttcataactaatttagatctctctccaatcaactaaggaaatcaactcttattaatagactgggccacacatctactaggcatgtaataaatgcttgctgaatgaacaaatgaatgaagagcctatagcatcatgttacagccatagtcctaaagtggtgtttctcatgaaggccaaatgctaagggattgagcttcagtcctttttctaacatcttgttctctaacagaattctcttcttttcttcataggagatgcctgagatacccaaaaccatcacaggtagtgagaccaacctcctcttcttctgggaaactcacggcactaagaactatttcacatcagttgcccatccaaacttgtttattgccacaaagcaagactactgggtgtgcttggcaggggggccaccctctatcactgactttcagatactggaaaaccaggcgtaggtctggagtctcacttgtctcacttgtgcagtgttgacagttcatatgtaccatgtacatgaagaagctaaatcctttactgttagtcatttgctgagcatgtactgagccttgtaattctaaatgaatgtttacactctttgtaagagtggaaccaacactaacatataatgttgttatttaaagaacaccctatattttgcatagtaccaatcattttaattattattcttcataacaattttaggaggaccagagctactgactatggctaccaaaaagactctacccatattacagatgggcaaattaaggcataagaaaactaagaaatatgcacaatagcagttgaaacaagaagccacagacctaggatttcatgatttcatttcaactgtttgccttctgcttttaagttgctgatgaactcttaatcaaatagcataagtttctgggacctcagttttatcattttcaaaatggagggaataatacctaagccttcctgccgcaacagttttttatgctaatcagggaggtcattttggtaaaatacttctcgaagccgagcctcaagatgaaggcaaagcacgaaatgttattttttaattattatttatatatgtatttataaatatatttaagataattataatatactatatttatgggaaccccttcatcctctgagtgtgaccaggcatcctccacaatagcagacagtgttttctgggataagtaagtttgatttcattaatacagggcattttggtccaagttgtgcttatcccatagccaggaaactctgcattctagtacttgggagacctgtaatcatataataaatgtacattaattaccttgagccagtaattggtccgatctttgactcttttgccattaaacttacctgggcattcttgtttcattcaattccacctgcaatcaagtcctacaagctaaaattagatgaactcaactttgacaaccatgagaccactgttatcaaaactttcttttctggaatgtaatcaatgtttcttctaggttctaaaaattgtgatcagaccataatgttacattattatcaacaatagtgattgatagagtgttatcagtcataactaaataaagcttgcaacaaaattctctgacacatagttattcattgccttaatcattattttactgcatggtaattagggacaaatggtaaatgtttacataaataattgtatttagtgttactttataaaatcaaaccaagattttatatttttttctcctctttgttagctgccagtatgcataaatggcattaagaatgataatatttccgggttcacttaaagctcatattacacatacacaaaacatgtgttcccatctttatacaaactcacacatacagagctacattaaaaacaactaataggccaggcacggtggctcagacctgtaatcccagcactttgggagg

SEQ ID NO: 2 - DNA, 9721 bp, Homo sapiens
  misc_feature (135)..(136): n is a, c, g, or t
agaaagaaagagagagagaaagaaaagaaagaggaaggaaggaaggaaggaagaaagacaggctctgaggaaggtggcagttcctacaacgggagaaccagtggttaatttgcaaagtggatcctgtggaggcanncagaggagtcccctaggccacccagacagggcttttagctatctgcaggccagacaccaaatttcaggagggctcagtgttaggaatggattatggcttatcaaattcacaggaaactaacatgttgaacagcttttagatttcctgtggaaaatataacttactaaagatggagttcttgtgactgactcctgatatcaagatactgggagccaaattaaaaatcagaaggctgcttggagagcaagtccatgaaatgctctttttcccacagtagaacctatttccctcgtgtctcaaatacttgcacagaggctcactcccttggataatgcagagcgagcacgatacctggcacatactaatttgaataaaatgctgtcaaattcccattcacccattcaagcagcaaactctatctcacctgaatgtacatgccaggcactgtgctagacttggctcaaaaagatttcagtttcctggaggaaccaggagggcaaggtttcaactcagtgctataagaagtgttacaggctggacacggtggctcacgcctgtaatcccaacatttgggaggccgaggcgggcagatcacaaggtcaggagatcgagaccatcctggctaacatggtgaaaccctgtctctactaaaaatacaaaaaattagccgggcgttggcggcaggtgcctgtagtcccagctgctggggaggctgaggcaggagaatggtgtgaacccgggaggcggaacttgcagggggccgagatcgtgccactgcactccagcctgggcgacagagtgagactctgtctcaaaaaaaaaaaaaaagtgttatgatgcagacctgtcaaagaggcaaaggagggtgttcctacactccaggcactgttcataacctggactctcattcattctacaaatggagggctcccctgggcagatccctggagcaggcactttgctggtgtctcggttaaagagaaactgataactcttggtattaccaagagatagagtctcagatggatattcttacagaaacaatattcccacttttcagagttcaccaaaaaatcattttaggcagagctcatctggcattgatctggttcatccatgagattggctagggtaacagcacctggtcttgcagggttgtgtgagcttatctccagggttgccccaactccgtcaggagcctgaaccctgcataccgtatgttctctgccccagccaagaaaggtcaattttctcctcagaggctcctgcaattgacagagagctcccgaggcagagaacagcacccaaggtagagacccacaccctcaatacagacagggagggctattggcccttcattgtacccatttatccatctgtaagtgggaagattcctaaacttaagtacaaagaagtgaatgaagaaaagtatgtgcatgtataaatctgtgtgtcttccactttgtcccacatatactaaatttaaacattcttctaacgtgggaaaatccagtattttaatgtggacatcaactgcacaacgattgtcaggaaaacaatgcatatttgcatggtgatacatttgcaaaatgtgtcatagtttgctactccttgcccttccatgaaccagagaattatctcagtttattagtcccctcccctaagaagcttccaccaatactcttttcccctttcctttaacttgattgtgaaatcaggtattcaacagagaaatttctcagcctcctacttctgcttttgaaagctataaaaacagcgagggagaaactggcagataccaaacctcttcgaggcacaaggcacaacaggctgctctgggattctcttcagccaatcttcattgctcaagtatgactttaatcttccttacaactaggtgctaagggagtctctctgtctctctgcctctttgtgtgtatgcatattctctctctctctctctttctttctctgtctctcctctccttcctctctgcctcctctctcagctttttgcaaaaatgccaggtgtaatataatgcttatgactcgggaaatattctgggaatggatactgcttatctaacagctgacaccctaaaggttagtgtcaaagcctctgctccagctctcctagccaatacattgctagttggggtttggtttagcaaatgcttttctctagacccaaaggacttctctttcacacattcattcatttactcagagatcatttctttgcatgactgccatgcactggatgctgagagaaatcacacatgaacgtagccgtcatggggaagtcactcattttctcctttttacacaggtgtctgaagcagccatggcagaagtacctgagctcgccagtgaaatgatggcttattacaggtcagtggagacgctgagaccagtaacatgagcaggtctcctctttcaagagtagagtgttatctgtgcttggagaccagatttttcccctaaattgcctctttcagtggcaaacagggtgccaagtaaatctgatttaaagactactttcccattacaagtccctccagccttgggacctggaggctatccagatgtgttgttgcaagggcttcctgcagaggcaaatggggagaaaagattccaagcccacaatacaaggaatccctttgcaaagtgtggcttggagggagagggagagctcagattttagctgactctgctgggctagaggttaggcctcaagatccaacagggagcaccagggtgcccacctgccaggcctagaatctgccttctggactgttctgcgcatatcactgtgaaacttgccaggtgtttcaggcagctttgagaggcaggctgtttgcagtttcttatgaacagtcaagtcttgtacacagggaaggaaaaataaacctgtttagaagacataattgagacatgtccctgtttttattacagtggcaatgaggatgacttgttctttgaagctgatggccctaaacagatgaaggtaagactatgggtttaactcccaacccaaggaagggctctaacacagggaaagctcaaagaagggagttctgggccactttgatgccatggtattttgttttagaaagactttaacctcttccagtgagacacaggctgcaccacttgctgacctggccacttggtcatcatatcaccacagtcactcactaacgttggtggtggtggccacacttggtggtgacaggggaggagtagtgataatgttcccatttcatagtaggaagacaaccaagtcttcaacataaatttgattatccttttaagagatggattcagcctatgccaatcacttgagttaaactctgaaaccaagagatgatcttgagaactaacatatgtctaccccttttgagtagaatagttttttgctacctggggtgaagcttataacaacaagacatagatgatataaacaaaaagatgaattgagacttgaaagaaaaccattcacttgctgtttgaccttgacaagtcattttacccgctttggacctcatctgaaaaataaagggctgagctggatgatctctgagattccagcatcctgcaacctccagttctgaaatattttcagttgtagctaagggcatttgggcagcaaatggtcatttttcagactcatccttacaaagagccatgttatattcctgctgtcccttctgttttatatgatgctcagtagccttcctaggtgcccagccatcagcctagctaggtcagttgtgcaggttggaggcagccacttttctctggctttattttattccagtttgtgatagcctcccctagcctcataatccagtcctcaatcttgttaaaaacatatttctttagaagttttaagactggcataacttcttggctgcagctgtgggaggagcccattggcttgtctgcctggcctttgccccccattgcctcttccagcagcttggctctgctccaggcaggaaattctctcctgctcaactttcttttgtgcacttacaggtctctttaactgtctttcaagcctttgaaccattatcagccttaaggcaacctcagtgaagccttaatacggagcttctctgaataagaggaaagtggtaacatttcacaaaaagtactctcacaggatttgcagaatgcctatgagacagtgttatgaaaaaggaaaaaaaagaacagtgtagaaaaattgaatacttgctgagtgagcataggtgaatggaaaatgttatggtcatctgcatgaaaaagcaaatcatagtgtgacagcattagggatacaaaaagatatagagaaggtatacatgtatggtgtaggtggggcatgtacaaaaagatgacaagtagaatcgggatttattctaaagaatagcctgtaaggtgtccagaagccacattctagtcttgagtctgcctctacctgctgtgtgcccttgagtacacccttaacctccttgagcttcagagagggataatctttttattttattttattttattttgttttgttttgttttgttttgttttatgagacagagtctcactctgttgcccaggctggagtgcagtggtacaatcttggcttactgcatcctccacctcctgagttcaagcgattctccttcctcagtctcctgaatagctaggattacaggtgcaccccaccacacccagctaatttttgtatttttagtagagaaggggtttcgccatgttggccaggctggttttgaagtcctgacctaaatgattcatccacctcggcttcccaaagtgctgggattacaggcatgagccaccacgcctggcccagagagggatgatctttagaagctcgggattctttcaagccctttcctcctctctgagctttctactctctgatgtcaaagcatggttcctggcaggaccacctcaccaggctccctccctcgctctctccgcagtgctccttccaggacctggacctctgccctctggatggcggcatccagctacgaatctccgaccaccactacagcaagggcttcaggcaggccgcgtcagttgttgtggccatggacaagctgaggaagatgctggttccctgcccacagaccttccaggagaatgacctgagcaccttctttcccttcatctttgaagaaggtagttagccaagagcaggcagtagatctccacttgtgtcctcttggaagtcatcaagccccagccaactcaattcccccagagccaaagccctttaaaggtagaaggcccagcggggagacaaaacaaagaaggctggaaaccaaagcaatcatctctttagtggaaactattcttaaagaagatcttgatggctactgacatttgcaactccctcactctttctcaggggcctttcacttacattgtcaccagaggttcgtaacctccctgtgggctagtgttatgaccatcaccattttacctaagtagctctgttgctcggccacagtgagcagtaatagacctgaagctggaacccatgtctaatagtgtcaggtccagtgttcttagccaccccactcccagcttcatccctactggtgttgtcatcagactttgaccgtatatgctcaggtgtcctccaagaaatcaaattttgccacctcgcctcacgaggcctgcccttctgattttatacctaaacaacatgtgctccacatttcagaacctatcttcttcgacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaaggtaaatggaaacatcctggtttccctgcctggcctcctggcagcttgctaattctccatgttttaaacaaagtagaaagttaatttaaggcaaatgatcaacacaagtgaaaaaaaatattaaaaaggaatatacaaactttggtcctagaaatggcacatttgattgcactggccagtgcatttgttaacaggagtgtgaccctgagaaattagacggctcaagcactcccaggaccatgtccacccaagtctcttgggcatagtgcagtgtcaattcttccacaatatggggtcatttgatggacatggcctaactgcctgtgggttctctcttcctgttgttgaggctgaaacaagagtgctggagcgataatgtgtccatccccctccccagtcttccccccttgccccaacatccgtcccacccaatgccaggtggttccttgtagggaaattttaccgcccagcaggaacttatatctctccgctgtaacgggcaaaagtttcaagtgcggtgaacccatcattagctgtggtgatctgcctggcatcgtgccacagtagccaaagcctctgcacaggagtgtgggcaactaaggctgctgactttgaaggacagcctcactcagggggaagctatttgctctcagccaggccaagaaaatcctgtttctttggaatcgggtagtaagagtgatcccagggcctccaattgacactgctgtgactgaggaagatcaaaatgagtgtctctctttggagccactttcccagctcagcctctcctctcccagtttcttcccatgggctactctctgttcctgaaacagttctggtgcctgatttctggcagaagtacagcttcacctctttcctttccttccacattgatcaagttgttccgctcctgtggatgggcacattgccagccagtgacacaatggcttccttccttccttccttcagcatttaaaatgtagaccctctttcattctccgttcctactgctatgaggctctgagaaaccctcaggcctttgaggggaaaccctaaatcaacaaaatgaccctgctattgtctgtgagaagtcaagttatcctgtgtcttaggccaaggaacctcactgtgggttcccacagaggctaccaattacatgtatcctactctcggggctaggggttggggtgaccctgcatgctgtgtccctaaccacaagacccccttctttcttcagtggtgttctccatgtcctttgtacaaggagaagaaagtaatgacaaaatacctgtggccttgggcctcaaggaaaagaatctgtacctgtcctgcgtgttgaaagatgataagcccactctacagctggaggtaagtgaatgctatggaatgaagcccttctcagcctcctgctaccacttattcccagacaattcaccttctccccgcccccatccctaggaaaagctgggaacaggtctatttgacaagttttgcattaatgtaaataaatttaacataatttttaactgcgtgcaaccttcaatcctgctgcagaaaattaaatcattttgccgatgttattatgtcctaccatagttacaaccccaacagattatatattgttagggctgctctcatttgatagacaccttgggaaatagatgacttaaagggtcccattatcacgtccactccactcccaaaatcaccaccactatcacctccagctttctcagcaaaagcttcatttccaagttgatgtcattctaggaccataaggaaaaatacaataaaaagcccctggaaactaggtacttcaagaagctctagcttaattttcacccccccaaaaaaaaaaaattctcacctacattatgctcctcagcatttggcactaagttttagaaaagaagaagggctcttttaataatcacacagaaagttgggggcccagttacaactcaggagtctggctcctgatcatgtgacctgctcgtcagtttcctttctggccaacccaaagaacatctttcccataggcatctttgtcccttgccccacaaaaattcttctttctctttcgctgcagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaaagagagctgtacccagagagtcctgtgctgaatgtggactcaatccctagggctggcagaaagggaacagaaaggtttttgagtacggctatagcctggactttcctgttgtctacaccaatgcccaactgcctgccttagggtagtgctaagaggatctcctgtccatcagccaggacagtcagctctctcctttcagggccaatccccagcccttttgttgagccaggcctctctcacctctcctactcacttaaagcccgcctgacagaaaccacggccacatttggttctaagaaaccctctgtcattcgctcccacattctgatgagcaaccgcttccctatttatttatttatttgtttgtttgttttgattcattggtctaatttattcaaagggggcaagaagtagcagtgtctgtaaaagagcctagtttttaatagctatggaatcaattcaatttggactggtgtgctctctttaaatcaagtcctttaattaagactgaaaatatataagctcagattatttaaatgggaatatttataaatgagcaaatatcatactgttcaatggttctgaaataaacttcactgaagaaaaaaaaaaaagggtctctcctgatcattgactgtctggattgacactgacagtaagcaaacaggctgtgagagttcttgggactaagcccactcctcattgctgagtgctgcaagtacctagaaatatccttggccaccgaagactatcctcctcacccatcccctttatttcgttgttcaacagaaggatattcagtgcacatctggaacaggatcagctgaagcactgcagggagtcaggactggtagtaacagctaccatgatttatctatcaatgcaccaaacatctgttgagcaagcgctatgtactaggagctgggagtacagagatgagaacagtcacaagtccctcctcagataggagaggcagctagttataagcagaacaaggtaacatgacaagtagagtaagatagaagaacgaagaggagtagccaggaaggagggaggagaacgacataagaatcaagcctaaagggataaacagaagatttccacacatgggctgggccaattgggtgtcggttacgcctgtaatcccagcactttgggtggcaggggcagaaagatcgcttgagcccaggagttcaagaccagcctgggcaacatagtgagactcccatctctacaaaaaataaataaataaataaaacaatcagccaggcatgctggcatgcacctgtagtcctagctacttgggaagctgacactggaggattgcttgagcccagaagttcaagactgcagtgagcttatccgttgacctgcaggtcgac

SEQ ID NO: 3 - DNA, 12565 bp, Homo sapiens
gtcgacctgcaggtcaacggatctgagaggagagtagcttcttgtagataacagttggattatataccatgtcctgatccccttcatcatccaggagagcagaggtggtcaccctgatagcagcaagcctgggggctgcagcttggtgggtagaggtactcaggggtacagatgtctccaaacctgtcctgctgccttagggagcttctaataagttgatggatttggttaaaattaacttggctacttggcaggactgggtcagtgaggaccaacaaaaagaagacatcagattataccctgggggtttgtatttcttgtgtttctttctcttctttgtactaaaatatttacccatgactgggaaagagcaactggagtctttgtagcattatcttagcaaaaatttacaaagtttggaaaacaatattgcccatattgtgtggtgtgtcctgtgacactcaggattcaagtgttggccgaagccactaaatgtgagatgaagccattacaaggcagtgtgcacatctgtccacccaagctggatgccaacatttcacaaatagtgcttgcgtgacacaaatgcagttccaggaggcccaaatgaaaatgtttgtactgaaatttgttaaagcttcccgacaaactagatttatcagtaaggattgttttctgcaagggggatgaaacttgtggggtgagccatttgggctgaggaggagggaggttggagctgagaaatgtggagacaatttccctttagaaggactgaatctccctgcctctctggggtgcggcagccagcaggatccaatggtgtatatgtctccccagctccccattcagtgatatcatgtcagtagcttgaaattatccgtggtgggagtattatgtcatggaaattggcaaatggaaacttttattggagattcaattgttaaacttttaccagcacaacactgccctgccttcagagtcaatgaccctatccaagtttaatccatctgtccactgtctccaacacgatctttataaaacacacctgacaacattacccttttattcagttttttaaaagataagtttccagctcatcggggtggctttaaaggccatttctcctctggacctcacccaacttttcaaatcacttttcctacccctacctctaaatgctactcaaactccagccatcctgaataataagacttttgaaaagtagattatgggctgggcacagtggctcacacctgtaatcccagcactttgggaggccaagatgggtggatcacctgaggtcgggagttcgagaccagcctgactaacatagtgaaaccctgtctctactaaaaatacaaaattagttgggggtggtggcacaagcctgtaatcccagctactcaggaggttgaggcaggggaattgcttgaacctgggaggcggaggttgcggtgagcctagattgctccactgcactccagcctgggcaacaagagcgaaactccatctcaaaaaaataaataaataaataaagtagattacatcagatacctctggcctaggttgtttatgaccaactctcctgctgagaataactagaaaagctagacaaaacatatttccaaaagatctctttggaggcatcagagaatggccaaggctgtaaggaactgcctgagcccagagaggtggagcccagcactggtgccctttactcctggggacatgtgctggtttcaaaaacttcagctgagcttttgagcattcatggaacttggtgggggagatgaaatttgtaccttaaatcctgcctacagggagggtccctgataatccccacccaatttggaaatctgggtcagccttcacaggtactgaagccctcctctgaatgatctcaagtcctgctagggtagaggttacctgcttttgaaaggctcctggcctacctgtgcagcaggagcaaaagtgaaccatctcagggtacagataacaatcatccagagccttgaatgacctctactgtgcttaatatatagtattcagcagtcagtaaaaaggatttaggcacatgcaagatgacctgtgtatcagggagaaataggcaataaattgagatccagcagggatttgaatcatggatttgaatcaggggcagccttcgaaagaactatggagaatatactcagatttaaaacataagattggaatttttggcagagaactaacaactgtacaaaaaaggaaccaaatggaaatcctagaactgaaagatgcaattaaccgatgttgagaaatagccaacatctattgaacacttcccatgtggacagctgtgctaaacactttacaggcatcaacataagatgtgtccccttacagcagtgcagtgtccctcctaagacatggacagcctggtttccctatctctctgcttcatcaaaacccctttacgtggggcttagacactcctgttgtctctagtgtctagtagcacagggctcagcacatggaagccactagatacaatttgatgaccaggacctccgatgaaagccatgggtgctgattgggaaggcattgtcttttatgtgctatggtcttaaagcttcatccaggaagcagaactcggggggtgctgaggacccagaaccgagaataagattagtcagagatttcctgtgggcagaaatcataaggacgccaactgtttgggtgagataagacgaaaccaagagtggacttgtggccagaagcgtgaggaagagggagagagcttcccttgtcccctttcttcctctccctaagccacagtgattgacagcccccccgctttggagtcagagcaggcttgagactggactgggaaaggagggtgggtcaggatacagagcaggaaggctgggagtgcagggcaggagcaaggggctggggcattcattgtgcctgatctctcccactttacctggggtaaagaagcatatgcaaaagccacggtgtgagtatttcccaagtgccagggtcagggcatgattcatcacgtgcagcatttcattcaatccttatagtaaccgatgatgtggcttctattattagctctatcagataatgaaactgagaccaagacaggctctgcacattgtgtggggtaatgacacagggggattcagacctagactccataactcctgccccagggaccacccccaccctcaccctgtgcatgtcgacaaaggacagactgggccacttctcaggacacagcggggaaatgacacagagcagggaggttccaggagccccgagcgtcttttctccaggagaatactctctgaattcagactggggtcagagaaacatttacccaggagccgcagtgtgggtggggctttttacttgaaacgctgtctgaaggcagtggcaggatgaactctccaccctaccttggcaagccacttctcttctgcaatctgtaaggacattgttgagagaattatggtcttccaattccggagggttgaagaaagacaaataggagagaacctatcatagtcaggtgctagctgccttctctttcagagagtgtgagaataaagtgatacacttgattattagcaaatactttggaaattttaaacgctaatattcaacacactctggaagaggcaaataagtagacaggttcatatacatcatctccttcagctagtcctcacaaaaacaaacaaatgaataaacaaaattcttctttggccctcataggaagacactgtttcttgaacgtgtttcaaaaaggatgggtgactcactcaaggtcacactgtttatgaggacagtacaggaatacagacatgccattttgcctgaaaaaatccatcacccagggaggtgacacaattttgcagaaatgttctatttcctctgaaggatacattctttaaacctttgggaaattcattcatagtcttcctcctttgaaggattactctctggacacaaagtgtttgattctgatttgttggttggaagatgtgttggttgagagaaagattctgatttgttggttgaaaatagactcatcaagatcaactgctgtagtagtaaatattttgacattttgtctgtattcctgtgctgccctcacaagctgcatcaccttgagtgagtcattcatacttttttgtttgtttttgttttggagatggagtcttactctgttgcctaggctggagtgcggtggcgtgatcttggctcactgcgacctccatctcctgggttcaagtgatcctcctgcctcagcctcccgagtagctgggattacaggcacatgccaccatccctgctaatttttgcattttcagtagagacggagtttcaccatgttggtcaggttggtcttgaactcctgacctcaggtgatccgcccacctcagcctccccaagtgctgggattacaggtgtgagccaccgtgcccagcccagccatcatttttgaaacacgtttgagaaatagtgtcttcctttgagggccaaggagacattttttttgtttatttgtttgtttttgtgaggactagctgaagggggtgatgtatattaacctgcctacttatttgcctcttcccagagtgtgatgaatattagggtttaaagtttctgaagcatttgttaataaagcccggggctggaggtcagaagacctggatttctctgcatacttttgccatcagcaagctgtgtgaccttggacagatcccttttttgtctaaatctttctgagtcttcttgaaaacaatgccaggttgggacaggatgattgccaagctcccgtccagctctaaaacactgcaacgtatgcttctgcaccagcactgtccatcctgtagatcatgcagaaattctcttcaactttttcctacccataaaataggagcatgcttacctttttcctaatgttccaggccccgggtctagatattgtaagtaaggaagttaatgtgtatcagagcccattatgggccagaagttctcctcttccttcctacacctgcttcctccctccctccctccctctttcccttccttccttccatccatttgtgaagaagacatgatcaccctcattctgagagtgaagagacagaggctcaactaatgaaatgatttgttcaaggtcacacgggtggcacaaggcaagtggcagaggttgaatttagacccattcctgtccaaatgctgagtttatgtcatcgtcccgagaccataactttaaagatgtaagatagtgggaaaagagttgatttcaaagcacctctcagaaggactcactttacatcaggggtcagcagactcaggccaaatccggtccattccccgcttttgcaaagaaagttgtagtggaacacagctaggcttattgatttatggattgccaacgtccttttgtgaaacagacagctgagctgagtaatcgtggcgcacaaaacctaaaatatttactatctcgtcctttacagaatgtttgccaatctatggtccggagtccaaggctgtccatttttcaaagaacacaaagtgacatgagactgtcccatgtgcagggagccctatcattttattatgaaaaaacggcctttctgctcaaatctgttttttaaaaagtcaacaaacagactctgggtacctgtcaggaacagtagggagtttggtttccattgtgctcttcttcccaggaactcaatgaaggggaaatagaaatcttaattttggggaaattgcacaggggaaaaaggggagggaatcagttacaacactccattgcgacacttagtggggttgaaagtgacaacagcaagggtttctctttttggaaatgcgaggagggtatttccgcttctcgcagtggggcagggtggcagacgcctagcttgggtgagtgactatttctttataaaccacaactctgggcccgcaatggcagtccactgcttgctgcagtcacagaatggaaatctgcagaggcctccgcagtcacctaatcactctcctcctcttcctgttccattcagagacgatctgccgaccctctgggagaaaatccagcaagatgcaagccttcaggtaaggctaccccaaggaggagaaggtgagggtggatcagctggagactggaaacatatcacagctgccagggctgccaggccagagggcctgagaactgggtttgggctggagaggatgtccattattcaagaaagaggctgttacatgcatgggcttcaggacttgtgtttcaaaatatcccagatgtggatagtgcgaccggagggctgtcttactttcccagagactcaggaacccagtgagtaatagatgcatgccaaggagtgggactgcgattcaggcctagttgaatgtgctgacagagaagcagagaggggcaccaggggcacagcccgaaggcccagactgatatgggcaaggcctgtctgtgctgacatgtcggagggtcccactctccagggaccttggtttccccgtctgtgacatctgtgacatgagagtcacgataactccttgtgtgccttacagggttgttgtgaaaattaaatgcacagataatagcgtaacagtattccgtgcattgtaaagagcctgaaaaccattatgatttgaaaatggaatcggctttgtgagaccatcactattgtaaagatgtgatgctgatagaaatgacaggactgcttgtgcatgccctctgcagtgtgacattccagcagtgaaatcatgttggggtgacttctcccccactctgacctttatgtttgtctgggccgaggctgcaagtcgggctctgtgggtgtatgagtgacaagtctctcccttccagatatggggactgtctgcttccctaggttgcctctccctgctctgatcagctagaagctccaggagatcctcctggaggccccagcaggtgatgtttatccctccagactgaggctaaatctagaaactaggataatcacaaacaggccaatgctgccatatgcaaagcactttggtttgcctggccacccctcgtcgagcatgtgggctcttcagagcacctgatgaggtgggtacagttagccacacttcacaggtgaagaggtgaggcacaggtcccaggtcaggctggccggagctctgtttattacgtctcacagctttgagtcctgctctcaaccagagaggccctttaccaagaagaaaggattgggacccagaatcaggtcactggctgaggtagagaggaagccgggttgttcccaagggtagctgctcctgcaggactctgagcaggtcaccagctaatggaggaaaggctctagggaaagacccttctggtctcagactcagagcgagttagctgcaaggtgttccgtctcttgaaacttctacctaggtgctatggtagccactagtctcaggtggctatttaaatttatacttaaatgaatgaaaatagaagaaaatttaaaatccagacccttggtcacactatccacatttaaagaggtcaatagccacatgtggttagtggccaccctattgggcagtgcagctacagaacatttttgcatcccagaaagttcttttggatgttgctgctctacagcatgctttgctgaaacagaagtgccttccctgggaatctcagatgggaagcaagtaaggaggggagtcaaatgtgggctcactgctcaccagctgtgagggttgggcctgcctcttaaccattgtcagcctcagtcttctcatccatgcatgccgtgggtatactaaaatactatacccctggaagagctggatgcaaatttgacaagttctgggggacacaggaaggtgccaagcacaaggctgggcacatggtggctgtgcactacagctgagtccttttccttttcagaatctgggatgttaaccagaagaccttctatctgaggaacaaccaactagttgctggatacttgcaaggaccaaatgtcaatttagaaggtgagtggttgccaggaaagccaatgtatctgggcatcacgtcactttgcccgtctgtctgcagcagcatggcctgcctgcacaaaccctaggtgcaatgtcctaatccttgttgggtctttgtattcaagtttgaagctgggagggcctggctactgaagggcacatatgagggtagcctgaagagggtgtggagaggtagagtctaggtcagaggtcagtgcctataggcaagtggtcccagggccacagctgggaagggcaaataccagaaggcaaggttgaccattcccttcctcaagtgcctattaaggctccatgttcctatgttgttcaaaccctaactcaatcccaaattaatccaccatgtataaggttgagctatgtctcttattcctggacaccatactcagccatatctggtccacacattaacagctggatgaccttgaagaagcttcacccactctgttcctcagctttcccttcagtgggatgatatcaactggacaacaggatgtgcgattcttttagttccagccttccaggatgttttcactcccctgtttgttgttgtaggatggtattacctccaccttcccaccttccctatgccctggttctgtctcctgtgcctcgctctgaaagtggatgagacctacaattcctgtcctggtagttctcctaatgaacacactgaagcacgaggaagctgagatttttgttgctacatgagagcatggaggcctcttagggagagaggaggttcagagactcctaggctcctggtggagccccactcatggccttgttcattttccctgcccctcagcaacactcctattgacctggagcacaggtatcctggggaaagtgagggaaatatggacatcacatggaacaacatccaggagactcaggcctctaggagtaactgggtagtgtgcatcctggggaaagtgagggaaatatggacatcacatggaacaacatccaggagactcaggcctctaggagtaactgggtagtgtgcatcctggggaaagtgagggaaatatggacatcacatggaacaacatccaggagactcaggcctctaggagtaactgggtagtgtgcatcctggggaaagtgagggaaatatggacatcacatggaacaacatccaggagactcaggcctctaggagtaactgggtagtgtgcttggtttaatcttctatttacctgcagaccaggaagatgagacctctctgcccttctgacctcgggattttagttttgtggggaccaggggagatagaaaaatacccggggtctcttcattattgctgcttcctcttctattaacctgaccctcccctctgttcttccccagaaaagatagatgtggtacccattgagcctcatgctctgttcttgggaatccatggagggaagatgtgcctgtcctgtgtcaagtctggtgatgagaccagactccagctggaggtaaaaacatgctttggatctcaaatcaccccaaaacccagtggcttgaaacaaccaaaattttttcttatgattctgtgggttgaccaggattagctgggtagttctgttccatgtggtggaacatgctggggtcactttggaagctgcattcagcagagtgccaggcttgcgctgggcatccaaggtggtccctcatcctccaggctctctttccatgtgatctctcagtgtttaagagttagttggagcttccttacagcatggcggctgacttccaaaagggattattccaaaaagagcctcaacatgcaggcgcttattatgacttctgcttgcatcatcctattggccaaagccagtcacgtggctaagtctagccccctgtgagaggagactgcataagagtgtgaacaccaggagacacggtcactgggggccaccactgtaaccatctaccacaggacctgaatctctgtgtgctactcccttgctcaagggcccccctacccacgcagacctgctgtcttctagcaaagcccatcctcaggacctttctcttccaatccttattgactcaaattgattagttggtgctccacccagagccctgtgctcctttatctcatgtaatgttaatgggtttcccagccctgggaaaacatggctttgtctcaggggcttgctggatgcaaccttaacctcaatgtgagtggccatactgtggcactgtcccatccctcaccagggacactgttctggagggtgactgcctgttctgtgaggagtggggatggctaggacattgcatggaacacaccaccaccccatcttctcagagctcaaaccctgacagaacaccagctccacaggccttggcttctgctgatggtgccgtgtatttaccagacttagtggtccaaggccagagtggcagatttcccaaagtcaaggtgtgacagtgggacagcctctttgtgtctttgctgtcctaagaaacctgggccaggccaggcgcagtggctcacgccttgtaatcccagcactttgagaggccaaggtgggcagatcacgaggtcaggagtttgagaccagcctggccaacattggtgaaaccctgtctctattaaaaatagaaaacattagacaggtgtggtggtgcatgcctgtaatcccagctactcaggaggctgaggcaggagaatcgcttgaacccaggaggtggaggttgcagtgagccgagattgtgccactgcactccagcctaggcgacagagcaagactccgtctcgggaaaattaattaataaataaataaacctaggtcccagagtcccacagaatggcagacaggagcacctgggggcttttagggtatggcatttcccctgtactaactctgggctgtccagaggcgatttcatggcgtggagtggagagggaggcagcacaggacttcctaggcctcagctctcacctgcccatcttttgatttccaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtagtactgcccaggcctgcctgttcccattcttgcatggcaaggactgcagggactgccagtccccctgccccagggctcccggctatgggggcactgaggaccagccattgaggggtggaccctcagaaggcgtcacaacaacctggtcacaggactctgcctcctcttcaactgaccagcctccatgctgcctccagaatggtctttctaatgtgtgaatcagagcacagcagcccctgcacaaagcccttccatgtcgcctctgcattcaggatcaaaccccgaccacctgcccaacctgctctcctcttgccactgcctcttcctccctcattccaccttcccatgccctggatccatcaggccacttgatgacccccaaccaagtggctcccacaccctgttttacaaaaaagaaaagaccagtccatgagggaggtttttaagggtttgtggaaaatgaaaattaggatttcatgatttttttttttcagtccccgtgaaggagagcccttcatttggagattatgttctttcggggagaggctgaggacttaaaatattcctgcatttgtgaaatgatggtgaaagtaagtggtagcttttcccttctttttcttctttttttgtgatgtcccaacttgtaaaaattaaaagttatggtactatgttagccccataattttttttttccttttaaaacacttccataatctggactcctctgtccaggcactgctgcccagcctccaagctccatctccactccagattttttacagctgcctgcagtactttacctcctatcagaagtttctcagctcccaaggctctgagcaaatgtggctcctgggggttctttcttcctctgctgaaggaataaattgctccttgacattgtagagcttctggcacttggagacttgtatgaaagatggctgtgcctctgcctgtctccccaccaggctgggagctctgcagagcaggaaacatgactcgtatatgtctcaggtccctgcagggccaagcacctagcctcgctcttggcaggtactcagcgaatgaatgctgtatatgttgggtgcaaagttccctacttcctgtgacttcagctctgttttacaataaaatcttgaaaatgcctatattgttgactatgtccttggccttgacaggctttgggtatagagtgctgaggaaactgaaagaccaatgtgtyttycttaccccagaggctggcgcctggcctcttctctgagagttcttttcttccttcagcctcactctccctggataacatgagagcaaatctctctgcgggg

SEQ ID NO: 4 - DNA, 2172 bp, Homo sapiens
gatcccacagctatagttcatggtgctgggatttgaacctctggccaccagagcccaccttaatgtgtcctcctcctgttgtcataacagaaaagtacaacaccatgatgacacatcaggctatcctggcaggttcccaggctgccccaatgcccaactttctaggtttacaaagttgacatttacgaagtttccaggtttacaaatctagtttctgattctttagtcagcaggaatttctctacaaaagctgcttcgaaaatttccagccaaaccttacacaccttggcaccacatcttggtgagccaaggcgaagagaacaggaagtgaaggccccatgggaagtccctgcggtcgggagcacccaggcggggcggggggtggggggctttcctgtggccggctccctgcccctcccacccccattcaggccctgtgagttgaatgaagagaccctgggaatgagtccaggtctgcagggttagaggaaattgaaggcccttaccagatccctgttgagaagtttatgaattatgagcccttctgcaaatgagagggttcttccctgtcaggagggacagattgtaggtggcaagattggtggcagccagtaggctggtctgctccttcctctctatttcatatgtgtatgaaggcattacctgcagcaagggcctgtgtaaatgcatgtgatttacagagcattttatgtactgcgtgtcattcatgcttccggtgagccctaagtctaagatagggcagatagcatcaggtccattttgcagctgtcaaaatgaggtctgaagggcagaagtggtgtgcccacacacacacaactggttggctgcagacctggggactagacccgggacttcgtcctgcccaggggtctcttgccactgctccccatcaacttggatggctttaagcatttgtgagttgtctgctccctgatggcagaatgcagagacatgaagctacaagcaggttcgctcccaacggcaaaaaggaggaggggtgttcagaacatcaggtgcttctagagaaagcagggagagagtatctggccttgtggacaatgtcacggcagaggccaggtatagggcatgggggtaactggaagcgggatggaccctcttattccctaagacatggcttccacgtagtgctcaaacaaggcctttgcccttgctgttccctccacctggaatattcttccccttccttgacattgctcaggtctccactcttatgtcaccctctcagagagggcttccctggccactttccctaaaatagccacccactcctaggtccctcaaaagcatatcctgctttggattttccctatagcaatatgccctatgaagttattttatttgctaacttgtttcttgtctgttttcctttgttagagcgttggggaccttgtctggcttgttcccaatgcctggaagagtgcctggcacacaggattaagccaacacatatgttttgaatgaatgtgtgcacacatgcatgagctggcggcagtcggggttggggtaagcacgaaggcccagctcagttctctgcatgtgacctcccatcttacgcagataagaaccagtttggtttctgctagcctgagtcaccctcctggaaactgggcctgcttggcatcaagtcagccatcagccggcccatctcctcatgctggccaaccctctgtgagtgtgtgggaggggaggctgggctcctccttgtactctctgaggtgctctggaaggaggggcagctccaccctgggagggactgtggcccaggtactgcccgggtgctactttatgggcagcagctcagttgagttagagtctggaagacctcagaagacctcctgtcctatgaggccctccccatggctttaggtaagctccttccactctcattttttcacctgagaaatgagagaggaaaatgtctacaattggtgtttatcaaatgctttcaggctctggtgagcaagcgtccaggaaaatgtcaagcgcatggagctccaggcctgtctgggggatctgggcacggggaggcatccatgggagaccatgcaggcactctgaggcaggggctgcaagctagtgcctgctggggcagcaggtgaacagagaggtgtaactgctgtgacagaagtc

SEQ ID NO: 5 - DNA, 30 bp, Homo sapiens
atggttttagaaatcatcaagcctagggca

SEQ ID NO: 6 - DNA, 30 bp, Homo sapiens
aatgaaaggaggggaggatgacagaaatgt

SEQ ID NO: 7 - DNA, 20 bp, Homo sapiens
tggcattgatctggttcatc

SEQ ID NO: 8 - DNA, 20 bp, Homo sapiens
gtttaggaatcttcccactt

SEQ ID NO: 9 - DNA, 25 bp, Homo sapiens
ctcaggtgtcctcgaagaaatcaaa

SEQ ID NO: 10 - DNA, 21 bp, Homo sapiens
gcttttttgctgtgagtcccg

SEQ ID NO: 11 - DNA, 17 bp, Homo sapiens
ctcagcaacactcctat

SEQ ID NO: 12 - DNA, 17 bp, Homo sapiens
tcctggtctgcagctaa

SEQ ID NO: 13 - DNA, 24 bp, Homo sapiens
ctatctgaggaacaaactagtagc

SEQ ID NO: 14 - DNA, 24 bp, Homo sapiens
taggacattgcacctagggtttgt

SEQ ID NO: 15 - DNA, 30 bp, Homo sapiens
atttttttataaatcatcaagcctagggca

SEQ ID NO: 16 - DNA, 30 bp, Homo sapiens
aattaaaggagggaagaatgacagaaatgt

SEQ ID NO: 17 - DNA, 27 bp, Homo sapiens
aagcttgttctaccacctgaactaggc

SEQ ID NO: 18 - DNA, 20 bp, Homo sapiens
ttacatatgagccttccatg

SEQ ID NO: 19 - DNA, 20 bp, Homo sapiens
tggcctccagaaacctccaa

SEQ ID NO: 20 - DNA, 20 bp, Homo sapiens
gctgatattctggtgggaaa

SEQ ID NO: 21 - DNA, 20 bp, Homo sapiens
ggcaagagcaaaactctgtc

SEQ ID NO: 22 - DNA, 27 bp, Homo sapiens
ctatctgaggaacaaccaactagtagc

SEQ ID NO: 23 - DNA, 25 bp, Homo sapiens
tgtacctaagcccaccctttagagc